SEQUENCE LISTING (19 sequences)

SEQ ID NO: 1 - RNA, 19 nt, Artificial Sequence
  Plasmid sequence
aauggauuuuuggagcagg

SEQ ID NO: 2 - RNA, 19 nt, Artificial Sequence
  Plasmid sequence
uggauuuuuggaucaggga

SEQ ID NO: 3 - RNA, 19 nt, Artificial Sequence
  Plasmid sequence
aauggauuuuuggagcagg

SEQ ID NO: 4 - RNA, 19 nt, Artificial Sequence
  Plasmid sequence
uggauuuuuggaucaggga

SEQ ID NO: 5 - DNA, 19 bp, Artificial Sequence
  Plasmid sequence
tattattcacatatttcac

SEQ ID NO: 6 - DNA, 19 bp, Artificial Sequence
  Plasmid sequence
ttttgcacttgcaggagcc

SEQ ID NO: 7 - DNA, 16 bp, Artificial Sequence
  Plasmid sequence
tgctccaaaaatccat

SEQ ID NO: 8 - DNA, 16 bp, Artificial Sequence
  Plasmid sequence
cctgatccaaaaatcc

SEQ ID NO: 9 - DNA, 15 bp, Artificial Sequence
  Plasmid sequence
acgtctatacgccca

SEQ ID NO: 10 - DNA, 19 bp, Artificial Sequence
  Probe sequence
cctgctccaaaaatccatt

SEQ ID NO: 11 - DNA, 19 bp, Artificial Sequence
  Probe sequence
tccctgatccaaaaatcca

SEQ ID NO: 12 - DNA, 22 bp, Artificial Sequence
  Probe sequence
gtgtaacacgtctatacgccca

SEQ ID NO: 13 - DNA, 22 bp, Artificial Sequence
  Probe sequence
cacgaatttgcgtgtcatcctt

SEQ ID NO: 14 - DNA, 16 bp, Artificial Sequence
  LNA
tgctccaaaaatccat

SEQ ID NO: 15 - DNA, 23 bp, Homo sapiens
aauagagugacccguaaaaucca

SEQ ID NO: 16 - DNA, 19 bp, Homo sapiens
ggacgagguuuuuagguaa

SEQ ID NO: 17 - DNA, 19 bp, homo sapiens
agggacuagguuuuuaggu

SEQ ID NO: 18 - DNA, 23 bp, homo sapiens
aatagagtgacccgtatttagca

SEQ ID NO: 19 - DNA, 23 bp, homo sapiens
aatagagtgacccgtaaaatcca

(19) United States
(12) Patent Application Publication
(10) Pub. No.: US 2020/0325539 A1
(43) Pub. Date: Oct. 15, 2020

(54) CANCER BIOMARKERS AND METHODS OF USE

(71) Applicant: AGENCY FOR SCIENCE, TECHNOLOGY AND RESEARCH, Singapore (SG)

(72) Inventors: Bing LIM, Singapore (SG); Wencai ZHANG, Singapore (SG); Wai Leong TAM, Singapore (SG)

(21) Appl. No.: 16/092,424

(22) PCT Filed: Apr. 6, 2017
(86) PCT No.: PCT/SG2017/050197

§ 371 Date: Oct. 9, 2018

(30) Foreign Application Priority Data
Apr. 6, 2016 (SG) 10201602700P

(51) Int. Cl. C12Q 1/6886; C12N 15/113; A61K 9/00

(52) U.S. Cl. CPC C12Q 1/6886; C12N 15/113; A61K 9/0019; C12N 2310/3231; C12Q 2600/178; C12Q 2600/118; C12N 2310/113; C12Q 2600/158

(57) ABSTRACT

The present disclosure relates to a method of determining the presence of lung cancer in a subject comprising the steps of detecting an expression level of miR-1246 alone or in combination with an expression level of miR-1290 in a sample obtained from the subject wherein the increase in miR-1246 and/or miR-1290 in the sample obtained from the subject relative to the expression level of miR-1246 or miR-1290 in the control sample indicates the presence of lung cancer in the subject. The disclosure further comprises a method of monitoring a response to therapy in a lung cancer patient, a method of prognosis of lung cancer in a patient and a method for treating lung cancer in a subject comprising the use of inhibitors of miR-1246 and/or miR-1290 alone or in combination. In a specific embodiment, miR-1246 and/or miR-1290 levels are used to predict response to chemotherapy and progression in human non-small cell lung cancer (NSCLC) patients.

### CROSS-REFERENCE TO RELATED APPLICATIONS

This application claims the benefit of priority of Singapore application No. 10201602700P, filed 6 Apr. 2016, the contents of it being hereby incorporated by reference in its entirety for all purposes.

### FIELD OF THE INVENTION

The invention is in the field of cancer biomarkers, in particular microRNAs as biomarkers for cancer.

### BACKGROUND OF THE INVENTION

Cancer is a class of diseases characterized by a group of cells that has lost its normal control mechanisms resulting in unregulated growth. Lung cancer is the deadliest cancer worldwide, with non-small cell lung cancer (NSCLC) and small-cell lung cancer accounting for approximately 85% and 15% of the incidences, respectively. Despite advances in detection and improvements to standard of care, NSCLC is often diagnosed at an advanced stage and bears poor prognosis. Relapses are frequent after primary and adjuvant therapy, often evolving into a lethal metastatic disease. These observations can, in part, be attributed to the highly heterogeneous nature of lung tumours that contain distinct tumoural and microenvironmental cell types, all of which contribute in varying degrees toward self-renewal, drug resistance, metastasis and relapse.

The tumour-initiating cell (TIC; also referred as cancer stem cell) model provides one explanation for the phenotypic and functional diversity among cancer cells in some tumours. TICs have been demonstrated to be more resistant to conventional therapeutic interventions, and are key drivers of relapse and metastasis. There is, therefore, increasing interests in developing strategies that can specifically target TICs with novel and emerging therapeutic modalities, thereby halting cancer progression and improving disease outcome.

MicroRNAs (miRNAs) represent a class of therapeutic targets that have been shown extensively to drive or inhibit cancer progression, and in some instances, may also be utilized as non-invasive biomarkers. A few studies have begun to demonstrate the contribution of miRNAs in TICs either using cultured human cell lines or mouse models, but these do not necessarily recapitulate their bona fide function in human tumours which tend to be more heterogeneous, and for which TICs can be better defined. Thus, there is a need to adopt the use of patient-derived tumour models and direct interrogation of patient materials for assessing the contributions of miRNAs and their diagnostic value in cancer.

### SUMMARY

In one aspect, there is provided a method for determining the presence of lung cancer in a subject, the method comprising: detecting an expression level of miR-1246 in a sample obtained from the subject; and comparing the expression level of miR-1246 in the sample to an expression level of miR-1246 in a control sample, wherein an increased expression level of miR-1246 in the sample obtained from the subject relative to the expression level of miR-1246 in the control sample indicates the presence of lung cancer in the subject.

In one aspect, there is provided a method of monitoring a response to therapy in a lung cancer patient, comprising: detecting an expression level of miR-1246, in a first sample obtained from the patient at a first time point, detecting the expression level of miR-1246, in one or more further samples obtained from the patient at one or more further time points, and comparing the expression level of miR-1246 detected at the first time point and one or more further time points, wherein the expression level of miR-1246 in the one or more further samples relative to the expression level of miR-1246 in the first sample, indicates the patient's response to therapy.

In one aspect, there is provided a method of prognosis of lung cancer in a patient, comprising: detecting an expression level of miR-1246 in a sample obtained from the subject; and comparing the expression level of miR-1246 in the sample to an expression level of miR-1246 in a control sample, wherein the expression level of miR-1246 in the sample obtained from the subject relative to the expression level of miR-1246 in the control sample indicates the prognosis of lung cancer in the subject.

In one aspect, there is provided a method for treating lung cancer in a subject, comprising administering to the subject one or more inhibitors of miR-1246.

In one aspect, there is provided a use of one or more inhibitors of miR-1246 in the manufacture of a medicament for treating lung cancer in a subject.

### Definitions

The term “sample” or “biological sample” as used herein refers to a cell, tissue or fluid that has been obtained from, removed or isolated from the subject. An example of a sample is a tumour tissue biopsy. Samples may be frozen fresh tissue, paraffin embedded tissue or formalin fixed paraffin embedded (FFPE) tissue. An example of samples include but is not limited to tissue, blood, serum, sputum, saliva, mucus, semen, plasma, urine, cerebrospinal fluid and bone marrow fluid.

The term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The terms “microRNA” and “miRNA” generally refer to a short, single stranded, non-coding ribonucleotide (RNA). MiRNA may encompass a region that is partially (between 10% and 50% complementary across length of strand), substantially (greater than 50% but less than 100% complementary across length of strand) or fully complementary to another region of the same single-stranded molecule or to another nucleic acid. Thus, a miRNA may encompass a molecule that comprises a self-complementary strand(s) or “complements” of a particular sequence comprising a molecule. For example, precursor miRNA may have a self-complementary region, which is up to 100% complementary. miRNA of the invention can include, can be or can be at least 60%, 65%, 70%, 75%, 80%, 85%, 90%, 95%, 96%, 97%, 98%, 99% or 100% complementary to their target. It will generally be understood that miRNAs typically bind to RNA targets, such as messenger RNA (mRNA). Binding of a miRNA to a mRNA may occur at coding or non-coding regions. Examples of non-coding regions that a miRNA may bind to are the 5′ or 3′ untranslated region (UTR). Binding of a miRNA to a target may suppress downstream functions of the target, such as translation. Binding of a miRNA to a target may also promote degradation of the target. Examples of miRNA include but are not limited to miR-1290, miR-1246, miR-130a, miR-130b, miR-196a, miR-196b, miR-630, miR-106b, miR-125b, miR-23a, miR-25, miR-320c, miR-3667-5p, miR-513-5p, miR-9*.

The term “inhibitor” as used in the context of miR-1290 or miR-1246 refers an agent that suppresses downregulates or silences the expression or activity of miR-1290 and/or miR-1246. It will be generally understood that the inhibitor may decrease or completely silence the expression or activity of miR-1290 and/or miR-1246. It will also be generally understood that the inhibitor may inhibit the expression or activity of miR-1290 and/or miR-1246 directly or indirectly. For example, direct inhibition may involve an inhibitor binding directly to the target miRNA. Indirect inhibition may involve interfering with one or more steps of miRNA assembly and function.

Examples of inhibitors of the microRNAs disclosed herein include but are not limited to oligonucleotides and small molecules. The term “oligonucleotide” generally refers to a single-stranded nucleotide polymer made of more than 2 nucleotide subunits covalently joined together. Preferably between 10 and 100 nucleotide units are present, most preferably between 12 and 50 nucleotides units are joined together. The sugar groups of the nucleotide subunits may be ribose, deoxyribose or modified derivatives thereof such as 2′-O-methyl ribose. An oligonucleotide may have uncommon nucleotides or non-nucleotide moieties. Oligonucleotides may also be synthetic or chemically modified. An oligonucleotide may also be an antisense oligonucleotide. It will be generally understood that an antisense oligonucleotide may have a nucleic acid sequence that is complementary to the nucleic acid sequence of a target (e.g. microRNA). Examples of antisense oligonucleotides include but are not limited to a Locked Nucleic Acid (LNA), an antisense mRNA and a morpholino.

The term “expression level” as used herein refers to the amount of gene, protein, or RNA (e.g. miRNA or shRNA) that is measurable in a sample. The expression level may be determined by quantifying RNA or protein levels. Examples of RNA include but are not limited to miRNA, shRNA, mRNA transcripts and spliced variants of mRNA transcripts. Examples of protein include but are not limited to proteins translated from the RNA, proteins that have been post-translationally modified and truncated proteins. Expression level may be absolute expression level or relative expression level that is relative to a reference, control or standard.

It will also be understood to one of skill in the art that a variety of detection agents and detection methods may be used to quantify expression levels. Examples of detection agents include but are not limited to primers, probes and complementary nucleic acid sequences that hybridise to the gene or protein. Detection methods may include conventional methods used in the art. Examples of detection methods include but are not limited to quantitative RT-PCR, in situ hybridization, microarray and sequencing.

The term “primer” is used herein to mean any single-stranded oligonucleotide sequence capable of being used as a primer in, for example, PCR technology. Thus, a “primer” according to the disclosure refers to a single-stranded oligonucleotide sequence that is capable of acting as a point of initiation for synthesis of a primer extension product that is substantially identical to the nucleic acid strand to be copied (for a forward primer) or substantially the reverse complement of the nucleic acid strand to be copied (for a reverse primer). A primer may be suitable for use in, for example, PCR technology.

The terms “reference”, “control” or “standard” as used herein refer to samples or subjects on which comparisons may be performed. Examples of a “reference”, “control” or “standard” include a non-cancerous sample obtained from the same subject, a sample obtained from a non-metastatic tumour, a sample obtained from a subject that does not have cancer or a sample obtained from a subject that has a different cancer subtype. The terms “reference”, “control” or “standard” as used herein may also refer to the average expression levels of a gene or protein in a patient cohort. The terms “reference”, “control” or “standard” as used herein may also refer to the average expression levels of a gene or protein in a cell line or plurality of cell lines. The terms “reference”, “control” or “standard” as used herein may also refer to a subject who is not suffering from cancer or who is suffering from a different type of cancer. An example of a reference is the average expression level of a gene in a patient cohort or the levels of average expression levels of the contrast cancer subtypes, e.g. small cell lung cancer.

The term “patient cohort” as used herein refers to a group of patients who share a common characteristic. Examples of patient cohorts are patients who are suffering from the same type of cancer. Patient cohorts may also comprise of patients that show the same clinical characteristics, including but not limited to survival and metastasis status at any given time point post disease diagnosis.

As used herein, the terms “increased expression level” or “decreased expression levels” and grammatical variants thereof refer respectively to higher or lower gene, RNA or protein expression levels relative to a reference. It will be generally understood that absolute quantification is not feasible for some detection methods for example, microarray, qRT-PCR based gene detection at mRNA level, or immunohistochemistry (IHC) based detection at protein levels. Rather, a skilled person would appreciate that a median level from a control cohort is used a reference sample.

The term “shRNA”, as used herein, refers to a short hairpin RNA which is a unimolecular RNA that is capable of performing RNAi and that has a passenger strand, a loop and a guide strand. The passenger and guide strand may be substantially complementary to each other. The term “shRNA” may also include nucleic acids that contain moieties other than ribonucleotide moieties, including, but not limited to, modified nucleotides, modified internucleotide linkages, non-nucleotides, deoxynucleotides, and analogs of the nucleotides mentioned thereof. shRNA is a single stranded RNA comprising a sequence and its complementary sequence separated by a stutter fragment which allows the RNA molecule to fold back on itself, creating a double stranded RNA molecule with a hairpin loop.

The term “response to therapy” as used herein, refers to a change in one or more identifiable disease states or outcomes. A disease state or disease outcome may be expression levels of one or more cancer markers (e.g. microRNA) in a sample, tumour metastasis, tumour size, survival rate, tumour recurrence or relapse, tumour invasion or death. Accordingly, “increased response to therapy” would be understood to mean that a subject or patient shows or is likely to show an improvement in disease state or outcome compared to a reference and “decreased response to therapy” would be understood to mean that a subject or patient shows or is likely to show a regression in disease state or outcome, or no change in disease state or outcome compared to a reference.

As used herein, the term “prognosis” or grammatical variants thereof refers to a prediction of the probable course and outcome of a clinical condition or disease. A prognosis of a patient is usually made by evaluating factors or symptoms of a disease that are indicative of a favorable or unfavorable course or outcome of the disease. The term “prognosis” does not refer to the ability to predict the course or outcome of a condition with 100% accuracy. Instead, the term “prognosis” refers to an increased probability that a certain course or outcome will occur; that is, that a course or outcome is more likely to occur in a patient exhibiting a given condition, when compared to those individuals not exhibiting the condition. For example, the course or outcome of a condition may be predicted with 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55% and 50% accuracy.

An example of prognosis is the likelihood of survival of a subject. Survival may be overall survival, distant metastasis free survival or relapse free survival. Other examples of prognosis include but are not limited to the likelihood of tumour metastasis and invasion, disease recurrence and death. Accordingly, a “poorer prognosis” would be understood to mean that that there is an increased probability compared to a reference that a patient will not survive, or that there is an increased probability of tumour metastasis, tumour invasion, disease recurrence or death. For example, a patient with a poorer prognosis has a lower chance of survival. In another example, a patient with a poorer prognosis is a patient with an increased likelihood of metastasis, disease recurrence or early death. Early death refers to the death of a patient post-diagnosis in a time period that is less than the time period of the death of patients in the reference sample.

### BRIEF DESCRIPTION OF THE DRAWINGS

The invention will be better understood with reference to the detailed description when considered in conjunction with the non-limiting examples and the accompanying drawings, in which:

FIG. 1 shows the heterogeneous expression of miR-1246 and miR-1290 in human NSCLC. (a) Tumour-formation efficiency of patient-derived tumour cell populations in NSG mice. T166+ or T166−, patient-derived CD166+ or CD166− sorted tumour cells. T166+, T166− and TS were generated from three patients. (b) Limiting dilution analysis of secondary tumour initiation by CD166+ and CD166− cells from primary xenograft tumours. Number of xenografted tumours formed and number of cells injected are shown. n=3 patient samples. (c) Intersection of miRNAs enriched in lung TICs (TS and T166+) compared with non-TICs (NHBE, SAEC and T166−) by miRNA microarray. Two panels with downregulated and upregulated miRNAs lists are shown. n=3. (d) qRT-PCR analysis of both miR-1246 and miR-1290 in non-tumorigenic cells (NHBE, SAEC and T166−) and TICs (T166+ and TS). n=3. (e) qRT-PCR analysis of miR-1246 and miR-1290 levels by box plot in paired tumour and normal tissues in NSCLC. The median values for miR-1246 and miR-1290 levels in normal tissues were normalized as 1; n=11. Differences between groups were analysed using unpaired t-tests. (f) Expression levels of miR-1246 in normal (N) and tumour (T) tissues across human lung adenocarcinoma (LUAD) and lung squamous carcinoma (LUSC) using TCGA miRNA-Seq data. Differences between groups were analysed using unpaired t-tests. n=521 (LUAD), n=504 (LUSC). (g) ISH for miR-1246 in primary lung adenocarcinoma. The staining intensity was classified as low/absent (0) and high (1+, 2+ and 3+). Both the staining intensity and number of tumours are shown. Scale bar, 50 mm. (h) Cumulative incidence estimates on the effect of miR-1246 and miR-1290 expression, as determined by ISH, on NSCLC mortality. The effect estimate was quantified in terms of the subdistribution hazard ratio and its associated 95% confidence interval (CI). The P values were calculated using log-rank tests; n=143. (i) Staining CD166 (immunohistochemistry, left) and miR-1246 (ISH, right) on serial sections of tumour and normal lung tissues. Scale bar, 50 μm. Data are represented as the mean±s.e.m. RQ, relative quantification; TS, patient-derived tumourspheres.

FIG. 2 shows miR-1246 and miR-1290 contribute towards transformation and lung tumorigenesis. (a) Sphere-formation assay for tumoursphere cells treated with either miR-1246 knockdown (zip1246) or miR-1290 knockdown (zip1290). A total of 500 cells were seeded into each 10-cm dish. Spheres containing >50 cells were counted on day 13. Scale bar, 200 μm. (b) Limiting dilution analysis of sphere-formation efficiency for tumoursphere cells treated with either zip1246 or zip1290. 50, 150 and 500 cells were plated; n=3. (c,d) Images (c) and quantitative analysis of volume and mass (d) of tumours formed 28 days after subcutaneous transplantation of 100,000 tumoursphere cells that were treated with either zip1246 or zip1290; n=6. (e) Limiting dilution analysis of tumour initiation by tumoursphere cells treated with either zip1246 or zip1290. In all, 100 and 2,000 cells were transplanted subcutaneously and tumour formation was evaluated 90 days later. The representative mouse images on transplantation (left) and the number of xenografted tumours/number of injections are shown; n=8. (f) qRT-PCR analysis of miR-1246 and miR-1290 expressions in HEK293 infected with either pre-miR-1246 (pre1246) or pre-miR-1290 (pre1290) overexpression. (g,h) Colony-formation assay (g) and quantification (h) in adherent conditions of HEK293 treated with either pre1246 or pre1290 overexpression. In all, 100 cells were plated; n=3. (i) Xenograft tumour-formation efficiency of HEK293 treated with either pre1246 or pre1290. A total of 100,000 cells were transplanted subcutaneously and tumour formation was evaluated 60 days later; n=6. (j,k) Soft-agar colony-formation assay (j) and quantification (k) in NuLi-1 treated with either pre1246 or pre1290. A total of 10,000 cells were plated and the colonies were stained by INT after 28 days. (l) Xenograft tumour-formation efficiency of NuLi-1 treated with either pre1246 or pre1290. A total of 1,000,000 cells were transplanted subcutaneously and tumour formation was evaluated 60 days later; n=6. (m) Xenograft tumour formation of CD166− tumour cells from xenograft tumour treated with pre1246 or pre1290. A total of 100,000 and 250,000 cells were transplanted subcutaneously and tumour formation was evaluated 90 days later; n=10. All error bars represent±s.e.m. and statistical significance was calculated using Student's t-test; *P<0.05, **P<0.01.

FIG. 3 shows the metastatic ability of lung TICs is dependent on miR-1246 and miR-1290. (a) ISH for miR-1246 and miR-1290 in paired primary lung adenocarcinoma and lymph node metastases. Scale bars, 600 mm (50 mm in the inset). (b) Association between miR-1246 or miR-1290 expression in primary tumours by ISH and status of lymph node (N) and distant metastases (M). (c,d) Transwell migration assay and quantification of tumoursphere cells treated with either zip1246 or zip1290. The migrated cells were stained with Giemsa (c) and counted 6 h after plating with 100,000 cells (d); n=3. Scale bars, 50 μm. (e) Fluorescence and bright-field images of mouse lung for assessing metastasis. A total of 1×10⁶ tumoursphere cells treated with either zip1246 or zip1290 were injected through the tail-vein. Lung metastatic nodules on day 13, 27 and 33 after transplantation are shown. Scale bars, 1 cm. (f-h) H&E (f) and E-cadherin immunohistochemistry staining (g) for mouse lung sections as shown in e (day 27). Quantitative analysis of the number of metastatic pulmonary nodules per lung is shown (h). Scale bar, 200 μm (f) and 50 μm (g); n=4. (i,j) H&E staining for mouse liver sections after injecting 1×10⁶ tumoursphere cells on day 27 via tail vein (i). Quantitative analysis of the number of metastatic nodules in the liver per mouse is shown (j). Nodules (see inset) are shown in higher magnification. Scale bar, 700 μm (inset, 200 mm); n=4. (k,l) Fluorescence and bright-field images (top panel) of the mouse lung for assessing metastasis. A total of 1×10⁶ tumoursphere cells treated with either zip1246 or zip1290 were injected subcutaneously. Whole-lung tissues were collected on day 60 (k) and until the subcutaneous tumours reached a similar size (12 mm in diameter) (l) after transplantation. The metastatic nodules are shown in higher magnification (see inset). Quantitative analysis of the number of metastatic lung nodules is shown (lower panel). n=4. Scale bars, 1 cm. All error bars represent±s.e.m. and statistical significance was calculated using Student's t-test; *P<0.05.

FIG. 4 shows longitudinal analyses of circulating miRNA levels in response to ongoing therapy in NSCLC patients. (a) qRT-PCR analysis of serum miR-1246 and miR-1290 levels in NSCLC patients and healthy individuals. Fold change of miR-1246 and miR-1290 levels is presented as box plot. The median value of serum miR-1246 or miR-1290 levels in healthy individuals was normalized to 1. Data are represented as mean±s.e.m. P values were calculated using Student's t-tests. T, NSCLC patients (n=59); N, healthy individuals (n=65). (b-d) Changes in serum miR-1246 and miR-1290 levels in response to therapy across multiple time points in NSCLC patients. The clinical disease progression status at various times was determined by CT (upper panel). All patients received EGFR TKI *, and in some instances, followed by either chemo or radiotherapy. The circulating miRNA levels are shown in the middle panel. Patients are categorized into four subgroups based on the pattern of clinical response to treatment. Representative response patterns from Group 1 (b) (response followed by progression; Patients 2 and 8), Group 2 (c) (progression; Patient 1), Group 3 (d) (stable disease; Patient 220) and Group 4 (e) (progression followed by response; Patients 218 and 219) are shown. The linear association between serum miRNAs levels and tumour size was analysed using Pearson's correlation coefficient (R) by SigmaPlot 11. Baseline CT scan was performed at day 0. EGFR TKI*=gefitinib and hydroxychloroquine.

FIG. 5 shows that administration of anti-miRNA LNA inhibitors in vivo inhibits tumour progression. (a) qRT-PCR analysis of miR-1246 and miR-1290 expression in tumoursphere cells on transfection with either LNA-antimiR-1246 or LNA-antimiR-1290. n=3. (b) Tumour initiation and growth measurements arising from the implantation of tumoursphere cells in NSG mice that were treated with LNA-antimiR-1246 or LNA-antimiR-1290 at either 8 mg kg⁻¹ or 2 mgkg⁻¹. A total of 100,000 tumoursphere cells were injected subcutaneously on day 0, and LNAs were administrated by intraperitoneal injection twice a week from day 0 to day 28. n=4. (c) Long-term xenograft tumour initiation and growth arising from the implantation of tumoursphere cells in NSG mice that were treated with LNA-antimiR-1246 or LNA-antimiR-1290. A total of 100,000 tumoursphere cells were injected subcutaneously on day 0, and LNAs were administrated by intraperitoneal injection twice a week from day 0 to day 49 at either 8 mg kg⁻¹ or 2 mg kg⁻¹. n=4. (d) Limiting dilution of analysis of tumour initiation from the implantation of tumoursphere cells in NSG mice that were treated with LNA-antimiR-1246 and LNA-antimiR-1290. A total of 100,000, 2,000 and 100 cells were injected subcutaneously on day 0, and LNAs were administrated by intraperitoneal injection twice a week from day 0 to day 90 at 8 mg kg⁻¹; n=4. (e) Effects of LNA treatment on the growth of pre-established tumours in NSG mice. A total of 100,000 tumoursphere cells were injected subcutaneously on day 0, and tumours were allowed to reach 5 mm diameter (˜30 days). Following that, LNAs were administrated by intraperitoneal injection twice a week from day 30 to day 49 at 8 mg kg⁻¹. n=4. (f) Images of xenograft tumours and quantitative analysis of tumour mass formed 19 days after administration of LNAs as shown in (e). n=4. (g) ELISA for serum albumin, ALT and AST levels in mice that were treated with 8 mg kg⁻¹ LNA-antimiR-1246 or LNA-antimiR-1290; n=4. (h) H&E staining of mice liver sections 7 weeks after administration of LNA-antimiR-1246 or LNA-antimiR-1290 at 8 mg kg⁻¹. Scale bar, 100 μm. All error bars represent±s.e.m. and statistical significance was calculated using Student's t-test; *P<0.05, **P<0.01.

FIG. 6 shows MT1G, a common target of miR-1246 and miR-1290, inhibits tumour growth and metastasis. (a,b) Venn diagram showing the identification for putative miR-1246 and miR-1290 target genes and validation by qRT-PCR. Genes (GE) upregulated (m) on zip1246 or zip1290 in A549 were overlapped with genes downregulated (k) on pre1246 or pre1290 in NuLi-1; n=3. (c) Computational prediction of duplex formations between miR-1246 or miR-1290 and the 3′-UTR of MT1G mRNA. Mutations generated within the 3′-UTR for the luciferase reported used in e are shown in red. (d) qRT-PCR analysis of metallothionein in TS and T166+ compared with N166+ and T166−; n=3. (e) Luciferase activity of wild-type (wt) or mutant (mut) MT1G 30-UTR reporter assay in HEK293 with pre1246 or pre1290; n=3. NS, not significant. (f) Immunohistochemistry staining of metallothioneins (low: 0; high: 1+−2+) for primary adenocarcinoma and normal lung tissue. Scale bar, 100 μm. (g,h) The associations between the intensity of metallothioneins expression (immunohistochemistry) and miR-1246 expression (ISH) (g) as well as CD166 expression (immunohistochemistry) (h) on a NSCLC tissue microarray. n=130 and 113 for g and h, respectively. (i) Western blot showing the overexpression of MT1G in TS. GAPDH was loaded as endogenous control. (j) Images and quantitative mass of tumours formed 54 days after subcutaneous transplantation of 1×10⁶ TS containing MT1G. n=6. (k) H&E staining and quantification of lung micrometastasis in mice bearing established tumours in j; n=6. Scale bar, 100 μm. (l) Images of lung whole mounts on day 34 following tail-vein injection of 1×10⁶ TS bearing MT1G; n=5. Scale bar, 1 cm. (m,n) Limiting dilution analysis of sphere formation in NuLi-1 overexpressing MT1G, and treated with pre1246 or pre1290 (m) and in TS bearing shMT1G, and treated with zip1246 or zip1290 (n). n=3. (o) Limiting dilution analysis of tumour formation 60 days after subcutaneous transplantation of 100 and 2000 TS bearing shMT1G, and treated with either zip1246 or zip1290. n=5. All error bars represent±s.e.m. For e, j, k, m, n, statistical significance was calculated using Student's t-test; for g,h, χ²-test. *P<0.05. **P<0.01.

FIG. 7 shows a miRNA signature enriched in TICs in NSCLC. (a) Flow cytometry analysis of CD166 and EPCAM in primary lung tumor (left). Isotype antibodies were applied as control (right). (b) Strategy for the identification and characterization of TIC-enriched miRNAs in NSCLC. (c) Quantitative RT-PCR analysis of candidate miRNAs listed in FIG. S1b in non-tumorigenic cells (NHBE, SAEC and T166−) and TICs (T166+ and TS) in NSCLC. Both downregulated miRNAs (left panel) and upregulated ones (right panel) are shown. The miRNAs level in NHBE was normalized as 1; n=3 replicates. (d) Quantitative RT-PCR analysis of miR-16, miR-92 and miR-26b, which are employed as endogenous controls, across primary NSCLC tumors and normal tissues. Ct, cycle threshold; n=9. (e) Quantitative RT-PCR analysis of miR-130b, miR-23a and miR-125b levels, presented as box plot, in paired tumor and normal tissues in NSCLC. The vertical line within the box indicates the median, boundaries of the box indicate the 25th- and 75th-percentile, and the whiskers indicate the highest and lowest values of the results. The median values in normal tissues were normalized as 1. N, normal; T, tumor; RQ, relative quantification; n=11. (f) Kaplan Meier curves showing the survival analyses of lung adenocarcinoma patients whose tumors express either high or low levels of miR-1246 and miR-1290 based on TCGA miRNA-Seq data. High or low expression was defined after performing normalization using the total numbers of mappable reads across all samples. TCGA, The Cancer Genome Atlas; n=397. (g) Correlation between expression of miR-1246 or miR-1290 and NSCLC stages based on TCGA miRNA-seq data. n=397. (h) ISH for miR-1246 in primary lung adenocarcinoma. Both scrambled miRNAs (left) and U6 (right) are applied as negative and positive controls, respectively. Scale bar, 50 μm. All error bars represent +SEM and statistical significance was calculated using Student's t-test (e,g) or log-rank test (f).

FIG. 8 shows expressions of CD166 and miR-1246 in lung tumors and normal tissue. (a) Co-staining CD166 (IHC) and miR-1246 (ISH) in lung tumors and normal tissues. Co-expression of CD166 (in brown) and miR-1246 (in purple) are shown in adenocarcinoma (T) and normal lung (N). Scale bar, 50 μm. (b) The association between the intensity of miR-1246 expression and miR-1290 expression by ISH on a NSCLC tissue microarray. p-value was calculated using Chi-squared test; n=169.

FIG. 9 shows the impact of mir-1246 or mir-1290 perturbations on in vitro growth. (a) Colony formation assay in adherent conditions of tumorsphere cells treated with either zip1246 or zip1290. 500 cells were plated. (b) Quantitative analysis of the number of colonies formed under adherent conditions in FIG. 9a. n=3 replicates. (c) Soft agar colony formation assay of tumorsphere cells that treated with either zip1246 or zip1290. 500 cells were plated. Colonies were stained with INT and counted on day 28. (d) Quantitative analysis of soft agar colony formation in FIG. S3c. n=3 replicates. (e) Proliferation assay of HEK293 cells treated with either pre1246 or pre1290. 100 cells were plated and the numbers of cells were evaluated on day 1, 2, 3 and 4 by CellTiter-Glo luminescent assay. n=3 replicates. All error bars represent +SEM and statistical significance was calculated using Student's t-test; *p<0.05, **p<0.01.

FIG. 10 shows miR-1246 or miR-1290 contribute towards lung cancer invasion and metastasis. (a) Transwell matrigel invasion assay of tumorsphere cells treated with either zip1246 or zip1290. 40,000 cells were seeded. The invaded cells on the lower membrane of transwell inserts were stained with Giemsa and counted 30 hours after plating. Scale bar, 50 μm. (b) Quantification of invasion assay as shown in FIG. S4a. n=3 replicates. All error bars represent +SEM and statistical significance was calculated using Student's t-test; **p<0.01. (c) H&E staining for mouse lung sections as shown in FIG. 3k on day 60, following the subcutaneous injection of 1×10⁶ tumorsphere cells that were treated with either zip1246 or zip1290. Sections of lung lobes in low (left) and high magnifications (right) are shown. Scale bar, 200 μm. (d) IHC staining for E-cadherin in primary lung adenocarcinoma. Staining pattern of E-cadherin (see inset) is shown in higher magnification. Scale bar, 50 μm.

FIG. 11 shows analysis for metastasis in patients with solid tumors including of lung adenocarcinoma based on the intensity of miRNAs expression in tumors. Heat maps showing miR-1246 and miR-1290 of the Gene Set Enrichment Analysis (GSEA) plots. The p-values were calculated with GSEA analysis. NES, normalized enrichment score; DN, down.

FIG. 12 shows expression levels of MT1G in primary lung cancer tissues and tumorsphere cells. (a) Quantitative RT-PCR analysis of MT1G in paired normal and cancerous tissues obtained from NSCLC patients. MT1G level in normal tissues was normalized as 1; n=9 paired tissues. (b,c) Transcriptome analyses showing downregulated gene expression in lung TICs (TS) compared with non-TICs (N166+). MT1G ranked among the top 50 of the 3,300 downregulated genes (b, c). TS, tumorsphere; N166+, CD166+ primary normal lung cells. n=3. (d) Luciferase activity of wild-type (UTR wt) or mutant (UTR mut) MT1G 3′ reporter assay in tumorsphere cells transfected with zip1246, zip1290 or zipCtrl. n=3 replicates. All error bars represent +SEM and statistical significance was calculated using Student's t-test; *p<0.05, **p<0.01; NS, not significant.

FIG. 13 shows MT1G suppresses the colony-forming and invasive abilities of NSCLC in vitro. (a) Colony formation assay in adherent conditions of tumorsphere cells overexpressing MT1G or control vector. 100 cells were plated. Colonies were stained with Giemsa (left panel) and quantified (right panel) on day 9; n=3 replicates. (b) Soft agar colony formation of tumorsphere cells overexpressing MT1G or control vector. 400 cells were seeded. Colonies as indicated (→) were stained with INT (left panel) and quantified (right panel) on day 28; n=3 replicates. Scale bar, 4 mm. (c) Transwell migration assay of tumorsphere cells overexpressing MT1G or control vector. 100,000 cells were seeded. The migrated cells on the lower membrane of Inserts were stained with Giemsa (left panel) and counted (right panel) 6 hours after plating; n=3 replicates. Scale bar, 400 μm. (d) Transwell matrigel invasion assay of tumorsphere cells overexpressing MT1G or control vector. 40,000 cells were seeded. The invaded cells on the lower membrane of transwell inserts were stained with Giemsa (left panel) and counted (right panel) 30 hours after plating; n=3 replicates. Scale bar, 400 μm. (e) Western blot showing the expression of metallothionein (MTs) protein in subcutaneous xenograft tumors obtained from mice (in FIG. 5f) that were treated with LNA-antimiR-1246, LNA-antimiR-1290 or control. α-tubulin was loaded as endogenous control. (f) The association between the intensity of MTs expression by IHC and status of regional lymph nodes (N) or distant metastasis (M) on a NSCLC tissue microarray. n=136. All error bars represent +SEM and statistical significance was calculated using Student's t-test (a, b, c, d) or Chi-squared test (f); *p<0.05, **p<0.01.

FIG. 14 shows enriched expression of miR-1246 and miR-1290 are found in a wide variety of tumor types relative to their normal tissue counterparts. (a) ISH index of miR-1246 expression level across different human tumor types (n=24, top panel) and normal tissues (n=30, bottom panel) on a tissue microarray (TMA). ISH index=expression intensity×expression as percentage. All error bars represent +SEM; n=3 replicates. ↓, lung. (b) ISH staining for miR-1246 in human tumors and normal tissues in two different TMAs. The candidate images showing miR-1246 expression in low (left panel) and high magnifications (right panel). N, normal. T, tumor. Scale bar, 600 μm (50 μm, inset). (c) ISH staining for miR-1246 in paired tumor and normal tissue samples from colon, esophagus, testis, skin and liver (top panels), and from breast, ovary, pancreas, kidney and spleen (bottom panels). Scale bar, 600 μm (50 μm, inset).

FIG. 15 shows expression levels of miR-1246 and miR-1290 across various human tumor types based on TCGA miRNA-Seq data. The plots showing the levels of miR-1246 (a) and miR-1290 (b) among up to 12 types of cancers relative to their normal tissues. p-values were calculated using Student's t-test.

FIG. 16 shows a schematic representation of the roles of miR-1246, miR-1290 and metallothioneins in NSCLC.

FIG. 17 shows uncropped Western Blots, corresponding to the indicated figures disclosed herein.

### DETAILED DESCRIPTION OF THE PRESENT INVENTION

In a first aspect the present invention refers to a method for determining the presence of lung cancer in a subject, the method comprising: detecting an expression level of miR-1246 in a sample obtained from the subject; and comparing the expression level of miR-1246 in the sample to an expression level of miR-1246 in a control sample, wherein an increased expression level of miR-1246 in the sample obtained from the subject relative to the expression level of miR-1246 in the control sample indicates the presence of lung cancer in the subject.

In some embodiments, the lung cancer may be non-small cell lung cancer (NSCLC) or small cell lung cancer (SCLC).

In a preferred embodiment, the lung cancer is a non-small cell lung cancer.

It will be generally understood in the art that NSCLC may include adenocarcinoma, squamous cell carcinoma or large cell carcinoma. Lung cancer may be characterized by stage, for example, Stage I, Stage II, Stage III, Stage IV, early stage, limited stage or extensive stage. Stages of lung cancer may be determined based on how far the cancer has spread. Stages of lung cancer may also be further divided into substages. In one embodiment, the lung cancer may be early stage lung cancer. In another embodiment, the lung cancer may be a stage I, II, III or IV lung cancer. It will also be generally understood that a lung cancer may be metastatic or non-metastatic lung cancer. In a preferred embodiment, the lung cancer may be a metastatic lung cancer.

The method of the present invention also discloses detecting an expression level of a gene or miRNA. In one embodiment, detecting may comprise one of quantitative RT-PCR, in-situ hybridization, microRNA microarray or microRNA sequencing. Combinations of the above may also be employed.

Detecting an expression level of a gene or miRNA may include detecting an increased expression level. In one embodiment, the increased expression level is between a 1 to 20-fold increase, such as a 1-fold, 2-fold, 3-fold, 4-fold or 5-fold increase.

The expression level of a gene or miRNA may be detected in a sample obtained from a subject. In one embodiment, the sample may be a selected from a tissue sample and a bodily fluid. In another embodiment, the sample may be selected from either a tissue sample or a bodily fluid.

In a preferred embodiment, the tissue sample may be a lung tissue sample. In another preferred embodiment, the bodily fluid sample may be selected from blood, urine, sputum, saliva, mucus, and semen. Combinations of samples may be possible. In a further preferred embodiment, the blood sample is a serum or plasma sample.

In one embodiment, the method of the present invention further comprises: detecting an expression level of miR-1290 in the sample obtained from the subject; and comparing the expression level of miR-1290 in the sample to an expression level of miR-1290 in a control sample, wherein an increased expression level of miR-1290 in the sample obtained from the subject relative to the expression level of miR-1290 in the control sample indicates the presence of lung cancer in the subject.

In another aspect of the invention, there is provided a method of monitoring a response to therapy in a lung cancer patient, comprising: detecting an expression level of miR-1246, in a first sample obtained from the patient at a first time point, detecting the expression level of miR-1246, in one or more further samples obtained from the patient at one or more further time points, and comparing the expression level of miR-1246 detected at the first time point and one or more further time points, wherein the expression level of miR-1246 in the one or more further samples relative to the expression level of miR-1246 in the first sample, indicates the patient's response to therapy.

The first sample and the one or more further samples may be the same type of sample or may be a different type of sample. For example, the first sample and the one or more further samples may be a tissue sample. In an alternative example, the first sample may be blood sample, and the further sample may be a lung tissue sample.

In one embodiment, the response may be monitored throughout the course of therapy. In another embodiment, the first time point may be prior to the start of therapy. In yet another embodiment, the one or more further time points are during the therapy and/or upon completion of the therapy.

In one embodiment, an increase in the expression level of miR-1246 in the one or more further samples relative to the expression level of miR-1246 in the first sample, indicates a decreased response to therapy.

In some embodiments, the increased expression level is in the one or more further samples between a 1 to 20-fold increase, such as a 1-fold, 2-fold, 3-fold, 4-fold or 5-fold increase.

In another embodiment, a decrease in the expression level of miR-1246 in the one or more further samples relative to the expression level of miR-1246 in the first sample, indicates an increased response to therapy.

In some embodiments, the decreased expression level is between a 1 to 20-fold decrease, such as a 1-fold, 2-fold, 3-fold, 4-fold or 5-fold decrease.

In some embodiments, the therapy is an anti-cancer therapy selected from the group consisting of a chemotherapeutic treatment, immunotherapy, tyrosine-kinase inhibitor (TKI) therapy, a surgical treatment, a treatment with radiation therapy or any combination thereof.

In other embodiments, the chemotherapeutic treatment comprises treatment with an antimetabolite, platinum complex, spindle poison, DNA crosslinking drug and alkylating agent, bleomycin, antibiotic, and topoisomerase inhibitor or combinations thereof.

In yet other embodiments, the tyrosine-kinase inhibitor (TKI) therapy comprises treatment with an EGFR tyrosine kinase inhibitor (TKI).

In one embodiment, the response to therapy is monitored in patient with non-small cell lung cancer.

In one embodiment, the method of monitoring a response to therapy in a lung cancer patient further comprises detecting an expression level of miR-1290, in the first sample obtained from the patient at the first time point, detecting an expression level of miR-1290, in the one or more further samples obtained from the patient at one or more further time points, and comparing the expression level of miR-1290 detected at the first time point and one or more further time points, wherein the expression level of miR-1290 in the one or more further samples relative to the expression level of miR-1290 in the first sample, indicates the patient's response to therapy.

The first sample and the one or more further samples may be the same type of sample or may be a different type of sample. For example, the first sample and the one or more further samples may be a tissue sample. In an alternative example, the first sample may be blood sample, and the further sample may be a lung tissue sample.

In one embodiment, the response may be monitored throughout the course of therapy. In another embodiment, the first time point may be prior to the start of therapy. In yet another embodiment, the one or more further time points are during the therapy and/or upon completion of the therapy.

In one embodiment, an increase in the expression level of miR-1290 in the one or more further samples relative to the expression level of miR-1290 in the first sample, indicates a decreased response to therapy. In some embodiments, the increased expression level is between a 1 to 20-fold increase, such as a 1-fold, 2-fold, 3-fold, 4-fold or 5-fold increase.

In one embodiment, a decrease in the level of miR-1290 in the one or more further samples relative to the expression level of miR-1290 in the first sample, indicates an increased response to therapy.

In some embodiments, the decreased expression level is between a 1 to 20-fold decrease, such as a 1-fold, 2-fold, 3-fold, 4-fold or 5-fold decrease.

In a further aspect of the present invention, there is provided a method of prognosis of lung cancer in a patient, comprising: detecting an expression level of miR-1246 in a sample obtained from the subject; and comparing the expression level of miR-1246 in the sample to an expression level of miR-1246 in a control sample, wherein the expression level of miR-1246 in the sample obtained from the subject relative to the expression level of miR-1246 in the control sample indicates the prognosis of lung cancer in the subject.

In one embodiment, an increased expression level of miR-1246 in the sample obtained from the subject relative to the expression level of miR-1246 in the control sample indicates any one of a decreased overall survival, a decreased progression-free survival, a decreased relapse-free survival, and/or a decreased distant-metastasis free survival

The increased expression level may be between a 1 to 20-fold increase, such as a 1-fold, 2-fold, 3-fold, 4-fold or 5-fold increase.

In one embodiment, the decreased expression level of miR-1246 in the sample obtained from the subject relative to the expression level of miR-1246 in the control sample indicates any one of an increased overall survival, an increased progression-free survival, an increased relapse-free survival, and/or an increased distant-metastasis free survival.

The decreased expression level is between a 1 to 20-fold decrease, such as a 1-fold, 2-fold, 3-fold, 4-fold or 5-fold decrease.

In one aspect of the present invention, there is provided a method for treating lung cancer in a subject, comprising administering to the subject one or more inhibitors of miR-1246.

In one embodiment, the one or more inhibitors of miR-1246 may comprise an antisense oligonucleotide specific for miR-1246. In a preferred embodiment, the antisense oligonucleotide is a Locked Nucleic Acid (LNA) specific for miR-1246.

In one embodiment, the one or more inhibitors of miR-1246 may be administered by any one of subcutaneous injection, intraperitoneal injection or intravenous injection.

In a further embodiment, the method for treating lung cancer in a subject may further comprise administering to the subject one or more inhibitors of miR-1290.

In some embodiments, the one or more inhibitors of miR-1290 comprise an antisense oligonucleotide specific for miR-1290.

In one aspect, there is provided a use of one or more inhibitors of miR-1246 in the manufacture of a medicament for treating lung cancer in a subject.

In some embodiments, the one or more inhibitors of miR-1246 comprise a LNA specific for miR-1246. In other embodiments, the medicament further comprises one or more inhibitors of miR-1290.

In some embodiments, the one or more inhibitors of miR-1290 comprise a LNA specific for miR-1290.

In another aspect, the present invention provides a composition comprising one or more inhibitors of miRNA-1246 and a physiologically acceptable carrier as disclosed herein.

In some embodiments, the composition may further comprise one or more inhibitors of miRNA-1290.

Compositions may include one or a combination of (e.g., two or more different) inhibitors of microRNAs of the invention. For example, a pharmaceutical composition of the invention can comprise a combination of inhibitors of miR-1290 and miR-1246.

Pharmaceutical compositions and medicaments of the invention also can be administered in combination therapy, i.e., combined with other agents. In some embodiments, the compositions and medicaments of the present invention may be administered with an anti-cancer therapy selected from the group consisting of a chemotherapeutic treatment, immunotherapy, tyrosine-kinase inhibitor (TKI) therapy, a surgical treatment, a treatment with radiation therapy, a targeted therapy or any combination thereof.

In some embodiments, the chemotherapeutic treatment may comprise treatment with an antimetabolite, platinum complex, spindle poison, DNA crosslinking drug and alkylating agent, bleomycin, antibiotic, and topoisomerase inhibitor or combinations thereof. In yet further embodiments, the anti-cancer therapy may be a further active pharmaceutical ingredient selected from the group consisting of bevacizumab, carboplatin, paclitaxel, hydroxychloroquine or gefitinib.

It will generally be understood that in combination therapy a first agent may be administered simultaneously, before, shortly before, after or shortly after administration of a second or subsequent agents. As used herein, shortly refers to 1 day, 12 hours, 6 hours, 5 hours, 4 hours, 3 hours, 2 hours, 1 hour, 30 minutes, 15 minutes, 5 minutes or 1 minute.

As used herein, “pharmaceutically acceptable carrier” or “physiologically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, oligonucleotide or inhibitor, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

The pharmaceutical compounds of the invention may include one or more pharmaceutically acceptable salts. A “pharmaceutically acceptable salt” or “physiologically acceptable salt” refers to a salt that retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline metals or alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and medicaments of the present invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The compositions and medicaments of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, the compositions or medicaments of the present invention can be administered via a non-parenteral route, such as a topical, epidermal or mucosal route of administration, for example, intranasally, orally, vaginally, rectally, sublingually or topically.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention, including the best mode, and a comparative example will be further described in greater detail by reference to specific Examples, which should not be construed as in any way limiting the scope of the invention.

Methods

Patients and Samples Collection.

Eligible patients were pathologically confirmed with the diagnosis of NSCLC. The solid tumours or serum were collected from patients according to protocols approved by the Ethics Committee of the National University of Singapore. Informed consent was obtained from the patients. Serum from stage I-III patients with NSCLC and healthy individuals was collected for serum miRNA profiling. To correlate serum miRNAs levels with therapy response, NSCLC patients at stage IIIB or IV, recruited as part of the NCT00809237 clinical study, took both 250 mg gefitinib, in combination with 600 mg hydroxychloroquine orally once daily, from the start of treatment to documented progression on CT imaging. Blood was drawn at every 4 weeks during treatment. CT was performed and documented in a blinded manner to monitor response to treatment according to the Response Evaluation Criteria in Solid Tumors (RECIST). This study has been approved by the National Healthcare Group Ethics committee (NHG IRB).

Cell Lines.

The A549 and HEK293 cell lines were obtained from ATCC and cultured in DMEM (high glucose; GIBCO) with 10% FBS, 2 mM L-glutamine and 1% penicillin-streptomycin. Primary NHBE and SAEC were obtained from Lonza and maintained in BEGM or SAGM complete-growth medium (Lonza). Immortalized NuLi-1 cell line was obtained from ATCC and cultured in BEGM serum-free complete-growth medium (Lonza) supplemented with 50 μgml⁻¹ G418 (Sigma).

Isolation of Single Tumour Cells.

All patients were first diagnosed with primary NSCLC without other tumour occurrences. They did not receive any therapy before surgery. Samples were chopped into small pieces, and incubated in 1 mgml⁻¹ collagenase/dispase solution (Roche, Indianapolis, Ind.) with 0.001% DNAse (Sigma-Aldrich, St Louis, Mo.) and 2% antibiotics (Sigma) in a water bath at 37° C. for 3 h. After incubation, the suspensions were passed through 70- and 40-m cell-strainers (BD Falcon, San Jose, Calif.) and centrifuged at 122 g for 5 min at 4° C. Cells were then resuspended in red blood cell lysis buffer (eBioscience, San Diego, Calif.) for 4 min at room temperature with intermittent shaking. The cell viability was evaluated by trypan blue dye exclusion. Live single cells accounted for 90% of the whole population.

Antibodies.

For fluorescence-activated cell sorting by flow cytometry, primary mouse anti-human CD166⁻ phycoerythrin (FAB6561P) was derived from R&D and anti-human EPCAM-FITC was derived from Miltenyi Biotech. For immunohistochemistry staining, primary rabbit anti-human E-Cadherin (1:500, cat #1702-1) was from Epitomics. Primary mouse anti-CD166 (1:50, clone MOG/07, NCL-CD166) was purchased from Novocastra (Leica Biosystem). Primary anti-human metallothionein (1:50, clone E9, M0639) was from Dako. Secondary goat anti-mouse antibody conjugated with horseradish peroxidase (HRP; 1:100, cat # P0447) and goat anti-rabbit antibody conjugated with HRP (1:100, cat # K4003) were from Dako. For western blot, primary rabbit anti-metallothionein antibody (1:300, clone FL-61, sc-11377) was from Santa Cruz. Mouse anti-GAPDH (1:5,000, Santa Cruz, sc-47724) and rabbit anti-α tubulin (1:2,000, Abcam, ab4074) were used as loading control. Secondary goat anti-mouse IgG H&L (HRP; 1:10,000, ab6789) and goat anti-rabbit IgG H&L (HRP; 1:10,000, ab6721) were purchased from Abcam. For ISH, sheep anti-digoxigenin (DIG) alkaline phosphatase (AP; cat #11093274910) came from Roche.

Fluorescence-Activated Cell Sorting.

For sorting, single-cell suspension was incubated with FcR blocking reagent (Miltenyi Biotech) in ice for 20 min. Then the cells were incubated with antibody against CD166 conjugated with phycoerythrin (R&D), and antibodies against lineage markers (human CD45 and CD31). To exclude dead cells, 7-amino-actinomycin D (BD PharminGen) was added before sorting. Appropriate isotype antibodies were used as controls.

Plasmids.

The MT1G 3′-UTR sequence was cloned into the pEZX-MT01 firefly/Renilla Duo-Luciferase reporter vector (GeneCopoeia). The pmiRZip-1246 and pmiRZip-1290 in miRZip-copGFP lentiviral vector (System Biosciences) to stably knockdown miR-1246 or miR-1290 expression was used following the manufacturer's instructions and contained the following shRNA sequence: 5′-AAUGGAUUUUUGGAGCAGG-3′ (SEQ ID NO:1) or 5′-UGGAUUUUUGGAUCAGGGA-3′ (SEQ ID NO:2), respectively. The pmiR-1246 and pmiR-1290 in pCDH-CMV-MCS-EF1-copGFP (CD511B-1) lentiviral vector (System Biosciences) to stably overexpress miR-1246 or miR-1290 was used following the manufacturer's instructions and contained the following sequence: 5′-AAUGGAUUUUUGGAGCAGG-3′ (SEQ ID NO:3) or 5′-UGGAUUUUUGGAUCAGGGA-3′ (SEQ ID NO:4), respectively. Precision pLOC-LentiORF-MT1G (OHS5899, Open Biosystems) and empty vector control Precision pLOC-LentiORF RFP (OHS5833) were used to overexpress MT1G. pTRIPZ inducible lentiviral shRNA against MT1G and pTRIPZ empty vector were used to knockdown MT1G. The sequences for shMT1G-1 and shMT1G-2 are 5′-TATTATTCACATATTTCAC-3′ (SEQ ID NO:5) and 5′-TTTTGCACTTGCAGGAGCC-3′ (SEQ ID NO:6), respectively. LNA inhibitors against miR-1246 or miR-1290 (Exiqon) contained the following sequence: 5′-TGCTCCA AAAATCCAT-3′ (SEQ ID NO:7) or 5′-CCTGATCCAAAAATCC-3′ (SEQ ID NO:8), respectively, and the scramble LNA inhibitor control's sequence was: 5′-ACGTCTATACGCCCA-3′ (SEQ ID NO:9).

Transient Transfection and Luciferase Assay.

PureFection (System Biosciences) was used for transient transfection. In all, 100 ng of wild-type or mutant 3′-UTR reporter constructs of MT1G constructs (GeneCopoeia) were cotransfected with 100 ng of pCDH-miR-1246, pCDH-miR-1290, pmiRZip-1246, pmiRZip-1290 or scrambled control vectors into HEK293 or tumoursphere cells. Firefly and Renilla luciferase activities were measured 48 h post-transfection using dual-luciferase reporter system (Promega). The firefly luminescence was normalized to Renilla luminescence as an internal control for transfection efficiency. MiR-1246- or miR-1290-binding site 5′-AAATC-3′ was substituted with 5′-TTTAG-3′ in mutated MT1G.

Microarray Assay.

Agilent Human miRNA Microarray Release 16.0, 8×60 K (G4872A-031181, Agilent Technologies) was used to identify miRNAs expressed in lung TICs. Total RNA (100 ng per sample) was hybridized to the microarrays. The miRNA expression profiles of tumour spheres, NSCLC patient-derived CD166⁺ and CD166⁻ xenograft tumour cells from three NSCLC patients were compared, as well as two normal lung epithelial cells, including NHBE and SAEC. MiRNA labelling, hybridization washing, scanning, feature extraction and application were carried out according to the manufacturer's instructions. All miRNA raw data were normalized based on the cross-correlation method. Significantly changed miRNAs were identified based on average fold change cutoff of 1.5 and the cutoff of the P value cross all replicates at 0.05.

HumanHT-12 v4 Expression BeadChip (Illumina) was used to identify the genes upregulated in A549 knocking down miR-1246 or miR-1290, and genes downregulated in NuLi-1 overexpressing miR-1246 or miR-1290. Total RNA (200 ng per sample) was hybridized to the microarrays. Total RNA was converted to double-stranded cDNA, followed by an amplification step to generate labelled cRNA. The following hybridization, image processing and raw data extraction were performed according to the manufacturer's instructions. All mRNA raw data were normalized based on the cross-correlation method. Significantly changed mRNAs were identified based on average fold change cutoff of 1.5 and the cutoff of the P value cross all replicates at 0.05.

MiR-1246 and miR-1290 Targets Prediction.

Potential miR-1246 and miR-1290 targets were identified by using two sets of wide-transcriptome microarray profiles: NuLi-1 relative to NuLi-1 cells overexpressing miR-1246 or miR-1290 (Illumina humanHT12_V4), and A549 relative to A549 cells knocking down miR-1246 or miR-1290 (Illumina humanHT12_V4). The following criteria were used to identify the possible miR-1246 or miR-1290 target genes: (1) genes downregulated 41.5-fold on miR-1246 or miR-1290 overexpression in NuLi-1 cells, and (2) genes upregulated 41.5-fold on miR-1246 or miR-1290 downregulation in A549 cells. All potential targets were subsequently verified by qRT-PCR.

Isolation and Quantification of Circulating Tumour miRNAs.

Whole blood was collected in red-topped tubes (BD). Blood was clotted by leaving it undisturbed at room temperature for 30 min. The clot was removed by centrifugation at 1,000 g for 10 min in a refrigerated centrifuge. Then the serum in the upper supernatant was transferred immediately into a clean tube for circulating miRNA assays.

Real-Time PCR for miRNAs and Genes.

Total RNA was extracted from solid tissues and cultured cells using mirVana miRNA Isolation Kit (Ambion) as well as from serum using mirVana PARIS kit (Ambion) according to the manufacturer's instructions. MiRNA expression was assessed by Tagman MicroRNA assay, and the gene expression of mRNAs was evaluated by Tagman Probes (Applied Biosystems). Taqman miRNA probes were as follow: hsa-miR-1246 (462575_mat), hsa-miR-1290 (002863), hsa-miR-130a (000454), hsa-miR-130b (000456), hsa-miR-196a (241070_mat), hsa-miR-196b (002215), hsa-miR-630 (001563), hsa-let-7b-5p (002619), hsa-let-7c (000379), hsa-let-7d-5p (002283), hsa-let-7i (002221), hsa-miR-106b (000442), hsa-miR-125b (000449), hsa-miR-23a (000399), hsa-miR-25 (000403), hsa-miR-320c (241053_mat), hsa-miR-3667-5p (462350_mat), hsa-513-5p (002090), hsa-miR-9* (002231). Taqman gene-expression probes were as follow: MT1G (Hs02578922_gH), MT1H (Hs00823168_g1), GLIPR1 (Hs01564143_m1), HAS2 (Hs00193435_m1), EVAlA (Hs00259924_m1), CYP4F11 (Hs01680107_m1), PRL36A (Hs01586542_g1), OSBPL6 (Hs00992951_m1), MAPK1 (Hs01046830_m1), YTHDC1 (Hs00180158_m1), AGBL5 (Hs00222447_m1), ZNF91 (Hs00602754_mH), PTK2 (Hs01056457_m1), NCKAP5 (Hs00418350_m1), GALNT13 (Hs00287613_m1). MiRNA expression was normalized to that of hsa-RNU48 (1006), miR-16 (000391), miR-26b (000407) and miR-92 (000430) (solid tissues), or hsa-miR-16 and hsa-miR-374 (000563) (cultured cells), or hsa-miR-425-5p (001516), hsa-RNU-48 and hsa-miR-16 (serum samples). Gene expression was normalized to GAPDH. Each sample was run in triplicate for real-time PCR.

Sphere-Formation Assay.

Single cells were resuspended in complete serum-free media. It contains DMEM/F12 with 50 ng ml⁻¹ epidermal growth factor (Invitrogen), 20 ng ml⁻¹ basic fibroblast growth factor (Invitrogen), 0.4% bovine serum albumin (Sigma), 0.05 mg ml⁻¹ insulin-transferring-selenium and 1% MEM non-essential amino acid (Gibco). Then cells were plated at 10,000 cells per well in six-well non-treated cell culture plates (Nunc). Fresh medium was replenished every 3 days. Tumourspheres were cultured for 10-14 days and then quantified. For passaging, tumourspheres were digested into single cells using accutase (Chemicon) and re-plated. For limiting dilution assay, 50, 150 and 500 of single cells were plated to assess sphere formation.

Colony-Formation Assay in Plate and Soft Agar.

Single cells were plated in 10-cm dishes in triplicates with 1,000 cells per dish. Fresh medium was replenished every 3 days. The cells were incubated for 10 days followed by Giemsa (Sigma) staining. The plates were air-dried and photographed, and the total number of colonies was analysed by openCFU (http://opencfu.sourceforge.net).

For soft-agar colony formation, 500 live cells were mixed with 0.35% top-agar and were plated onto 0.6% base-agar in six-well plates with triplicates. The cells were incubated for 14-21 days followed by INT staining overnight. The plates were photographed and the colony numbers were counted by Gelcount (Oxford optonix).

Cell Proliferation Assay.

Cultured cells were plated into 96-well plates with 400 HEK293 cells per well in four replicates on day 0. The cell viability was measured every day using CellTiter-Glo luminescent cell viability assay kit (Promega) according to the manufacturer's instructions. Luminescent signal was recorded as relative light units. The relative proliferation index on day 0 was normalized as 1.

Cell Migration and Invasion Assays.

In vitro cell migration and invasion assay were performed using Boyden chambers (BD bioscience) that use 8 mm micropore membranes without Matrigel (for migration assay) or with Matrigel (for invasion assay). Both assays were carried out according to the manufacturer's instructions. The cells were resuspended in 0.1% bovine serum albumin in DMEM/F12 medium and seeded in the upper chamber at a concentration of 1×10⁵/0.5 ml. The chambers were incubated in the wells containing DMEM/F12 medium with 10% FBS for 6 or 24 h. Filters were fixed with 3.7% formaldehyde and stained with Giemsa. The cells on the upper surface of the filters were removed by swabbing with a cotton swab, and the cells that had migrated to the reverse side were counted in 10 random fields under a microscope (Zeiss) at 400× magnification.

Lentiviral-Mediated miRNA and MT1G Overexpression or Knockdown.

Lentivirus was produced in 293FT packaging cells and collected 48-72 h post-infection. For lentiviral overexpression or knockdown of miR-1246 or miR-1290, cells (tumoursphere, A549, NuLi-1 and HEK293) were infected with the lentiviral supernatant for 48 h in the presence of 8 gml⁻¹ polybrene (Sigma). Two days after infection, puromycin was added to the media at 1 μgml⁻¹, and cell populations were selected for 1-2 weeks. For lentiviral overexpression of MT1G, cells (tumoursphere and HEK293) at 70% confluence were transduced with MT1G lentiviral particles (1.64×10⁹ TUml⁻¹, Open Biosystems) or control lentiviral particles (2×108 TUml⁻¹, Open Biosystems) together with polybrene. Then the infected cells were passaged and selected by blasticidin S (Invitrogen) at 12 mgml⁻¹ for 1-2 weeks. For inducible lentiviral knockdown of MT1G, tumoursphere cells at 70% confluence were transduced with two shRNAs against MT1G lentiviral particles (Open Biosystems) or control lentiviral particles together with polybrene. Then the infected cells were passaged, induced by 0.5 μg ml⁻¹ doxycycline and then selected by puromycin at 1 μgml⁻¹ for 1-2 weeks.

Western Blot.

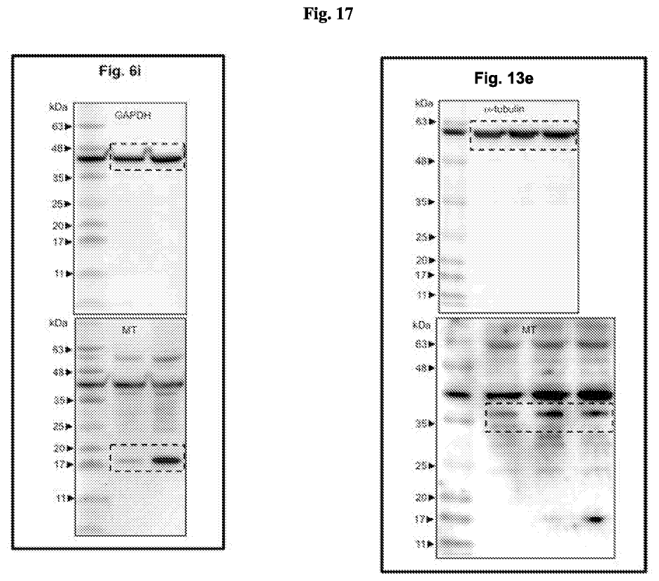

Cells were collected and lysed with Nonidet-P40 supplemented with protease inhibitor cocktail (Roche). Protein concentrations of the extracts were measured using BCA assay (Pierce) and equalized with the extraction reagent. Equal amount of the extracts was loaded and subjected to SDS-PAGE, transferred onto nitrocellulose membranes. Peroxidase-conjugated anti-mouse (1:10,000, ab6789) or rabbit IgG (1:10,000, ab6721) was used as secondary antibody and the antigen-antibody reaction was visualized by Amersham ECL Prime detection reagent (GE Healthcare). Uncropped Western Blot images are included in FIG. 17.

H&E and Immunohistochemistry.

Samples were formalin-fixed, paraffin embedded (FFPE), sectioned and stained with haematoxylin-eosin (H&E) according to standard histopathological techniques. For immunohistochemistry, sections were incubated with anti-human CD166 (Novaocastra), E-cadherin (Epitomics) and metallothioneins (Dako), and visualized using the Envision HRP Polymer System (Dako). All images were captured on a high-throughput Leica SCN400 scanner.

MiRNA ISH.

ISH on tissue microarray sections from FFPE tissue sample with human lung and other types of cancers was applied by using the miRCURY LNA microRNA ISH Optimization kit 5. Five micrometer-thick tissue sections were incubated with 15 μgml⁻¹ proteinase K for 45 min at 37° C. After washing, the sections were incubated with 20 nM LNA probes (50-double-digoxigenin-labelled LNA probes specific for human miR-1246 (5′-cctgctccaaaaatccatt-3′) (SEQ ID NO: 10), miR-1290 (5′-tccctgatccaaaaatcca-3′) (SEQ ID NO: 11) or scrambled probe (5′-gtgtaacacgtctatacgccca-3′) (SEQ ID NO: 12) (Exiqon)) in hybridization buffer (Roche) overnight at 55° C. After stringent washes, sections were blocked with 2% sheep serum and further incubated with sheep anti-digoxigenin AP (Roche; 1:500) at room temperature for 2.5 h. Sections were washed in PBS-T (0.1%) and miRNA-bound LNA probes were detected by AP substrate (Roche) at room temperature for 1.5 h. After counterstaining with Nuclear Fast Red (Vector laboratories), slides were mounted using mounting medium (Eukitt). Image acquisition was performed with high-throughput Leica SCN400 scanner and/or Olympus FluoView FV1000. LNA 50-digoxigenin-labelled (5′-cacgaatttgcgtgtcatcctt-3′) (SEQ ID NO: 13) U6 snRNA probe at 0.1 nM was used as positive control.

For co-localization of miRNA-1246/miR-1290 and CD166 protein in FFPE tissues, the sections were stained with LNA probe by ISH followed by immunohistochemistry with anti-CD166 (Novaocastra).

Tissue Microarray.

A tissue microarray with regional lymph nodes, malignant and cancer-adjacent normal lung specimens from NSCLC patients was constructed. Tumour specimens were transferred to the Department of Pathology, National University Hospital of Singapore within 1 h after surgical removal. Suitable areas for tissue retrieval were marked on standard H&E sections, punched out of the paraffin block and inserted into a recipient block. The punch diameter was 0.6 mm. The tissue array was cut in 4-μm thick sections. Tissue microarrays including multiple organs (FDA808b-1, 2 and BC00112) were purchased from Biomax. MiR-1246, miR-1290 or metallothionein staining was independently scored by two anatomical pathologists (M.E.N and Y.H.P). Staining intensity was scored semi-quantitatively (score 0: undetectable; 1+: weak; 2+: moderate; 3+: strong) and grouped as low (score 0) or high (scores 1+˜3+).

Transfection by LNAs In Vitro.

Tumoursphere cells were plated at 3,000 cells in serum-free medium in a 96-well non-treated plates to reach 50-60% confluence. In all, 50 nM of LNA anti-miR-1246, anti-miR-1290 or negative control (Exiqon) with fluorescine and PureFection (System Biosciences) were applied for transfection. The transfected cells were collected after culturing for 40 h.

Animal Studies.

All research involving animals complied with protocols approved by the A*STAR Biological Resource Centre Institutional Animal Care and Use Committee. Four to 6-week-old female NSG immunodeficient mice (Jackson Laboratory) were used for subcutaneous injections, and 6-8-week-old female NSG mice were used for tail-vein injections. For subcutaneous xenograft tumour assay and/or spontaneous metastasis assay, 100 to 1×10⁶ cells (CD166⁺ and CD166⁻ tumour cells, tumoursphere, HEK293 and NuLi-1) in serum-free medium and Matrigel (BD; 1:1) were inoculated into the flank of NSG mice. The xenograft tumour formation was monitored by calipers twice a week. The recipient mice were monitored and killed when the tumours reached 2 cm in diameter, and thus the metastases by tumoursphere cells were evaluated 60-90 days post transplantation. The subcutaneous xenograft tumours and the spontaneous metastasis into lung were analysed under a dissecting microscope equipped with GFP fluorescence imaging.

For the tail-vein assay of cancer metastasis, cells were inoculated intravenously into 6-8-week-old female NSG mice, and both the lungs and the liver were removed on the 13th, 27th and 33rd day post transplantation, and fixed with 10% neutral-buffered formalin. Tumour metastasis was recorded by fluorescence imaging. For quantitative analysis of metastasis, the metastatic lung nodules >0.4 mm were counted. Metastatic index was calculated as number of lung nodules per mouse/volume of subcutaneous tumour.

LNA Synthesis and Administration.

Custom-made miRCURY LNAs for in vivo application were designed and synthesized as unconjugated and fully phosphorothiolated oligonucleotides by Exiqon. The sequences of the LNA targeting miR-1246 or miR-1290 were fully complementary to the mature miRNA sequence: 5′-TGCTCCAAAAATCCAT-3′ (SEQ ID NO: 14) (LNA antimiR-1246) and 5′-CCTGATCCAAAAATCC-3′ (SEQ ID NO: 8) (LNA antimiR-1290); the scrambled LNA control was 5′-ACGTCTATACGCCCA-3′ (SEQ ID NO: 9) (LNA antimiR-ctrl). LNA was intraperitoneally delivered to mouse at a dose of 2 or 8 mgkg⁻¹ body weight in 1×PBS. At the beginning of tumour formation assay, mice were injected twice a week from day 0 on implantation of 1×10⁵, 2,000 and 100 tumoursphere cells via subcutaneous injection and killed 49-90 days after LNA administration. In mice with established subcutaneous xenograft tumours (5 mm in length), LNAs were administrated twice a week at 8 mgkg⁻¹ body weight for 19 days. Four mice were used in each group.

Enzyme-Linked Immunosorbent Assay.

Mouse serum albumin, ALT and AST levels were measured at different time points after LNA treatment by enzyme-linked immunosorbent assay according to the manufacturer's instructions (USCN).

Data Analysis.

For survival analysis and GSEA, two lung tumour (LUAD and LUSC) RNA-seq data sets were utilized from the TCGA data portal. The survival analysis was based on the Kaplan-Meier method for two sample groups of low and high miRNA expression. In segregation of patient samples into high and low groups, normalization using the total numbers of mappable reads across all samples was first performed. Then the miRNA mean expression as cutoff was applied to segregate high- and low-expression samples. In addition, those middle samples with expression close to the mean expression value were removed, since they might be equally classified into either group. For identification of enriched gene sets, GSEA was performed based on the normalized data and using GSEA v2.07 tool (http://www.broad.mit.edu/gsea/) with msigdb.v4.0.

Statistical Analysis.

Data are presented as the mean±s.e.m. Unless otherwise stated, statistical significance was determined by a Student's two-tailed t-test. P<0.05 was considered statistically significant. The associations between expressions of miRNAs or CD166 and metallothioneins were evaluated using χ²-test. The linear association between serum miRNAs levels and tumour size was analysed using Pearson's correlation coefficient (R) by SigmaPlot 11.

Results

Identification of miRNAs Restricted to TICs in NSCLC.

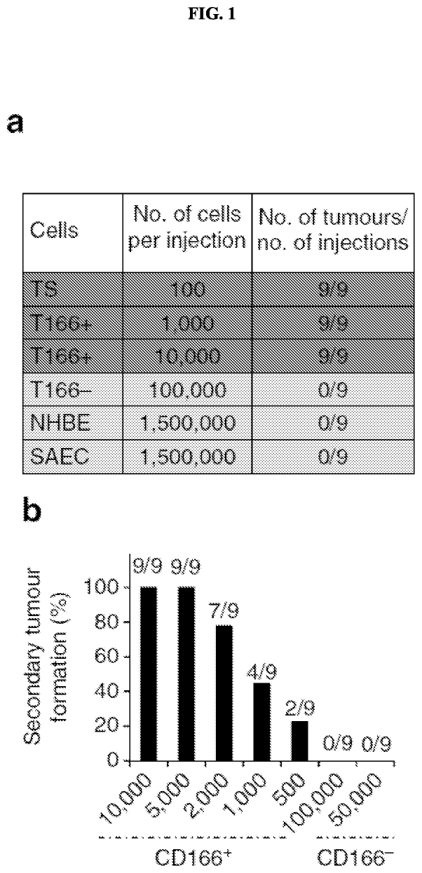

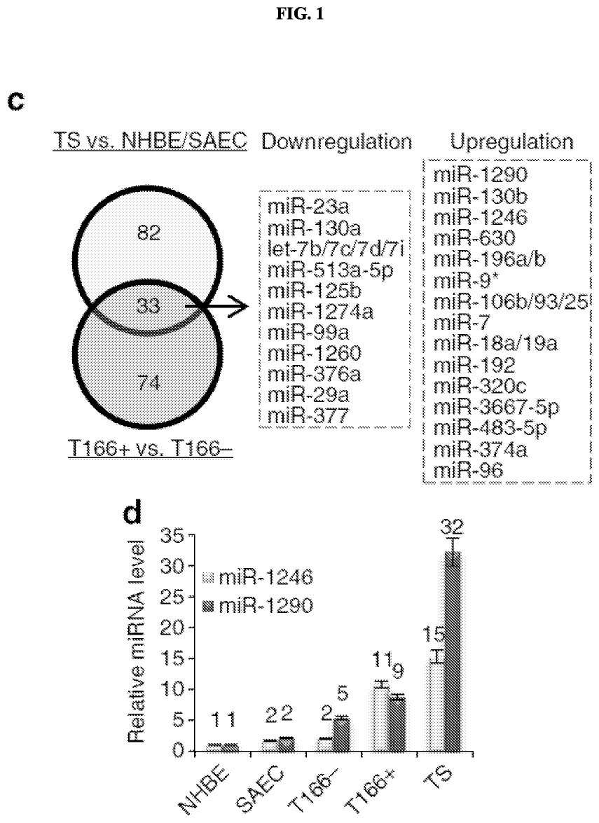

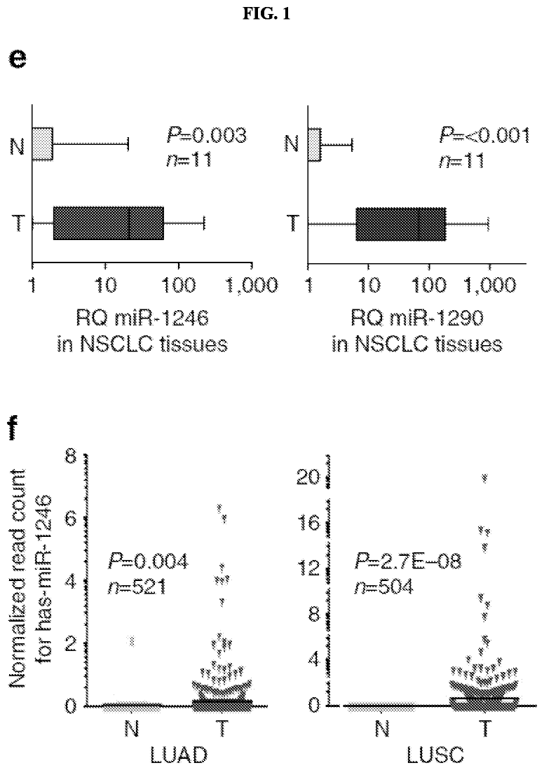

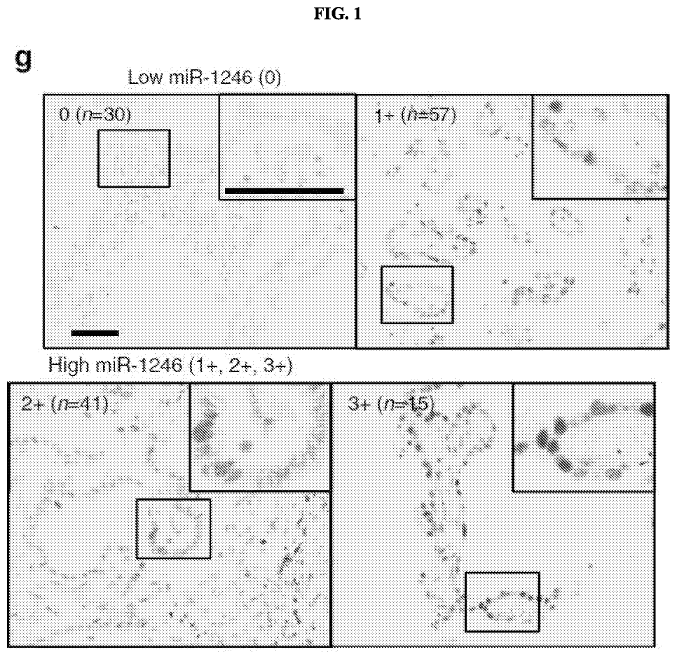

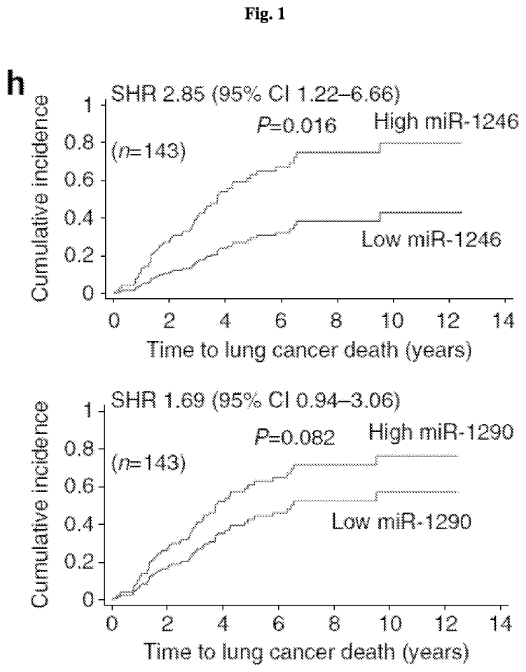

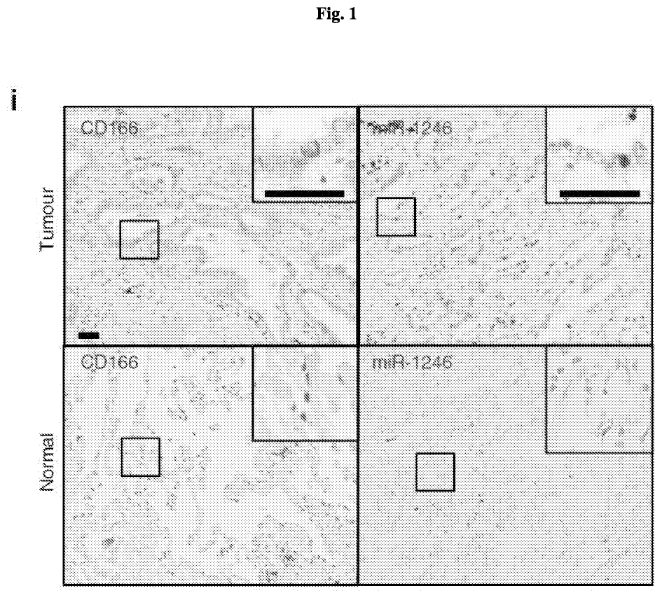

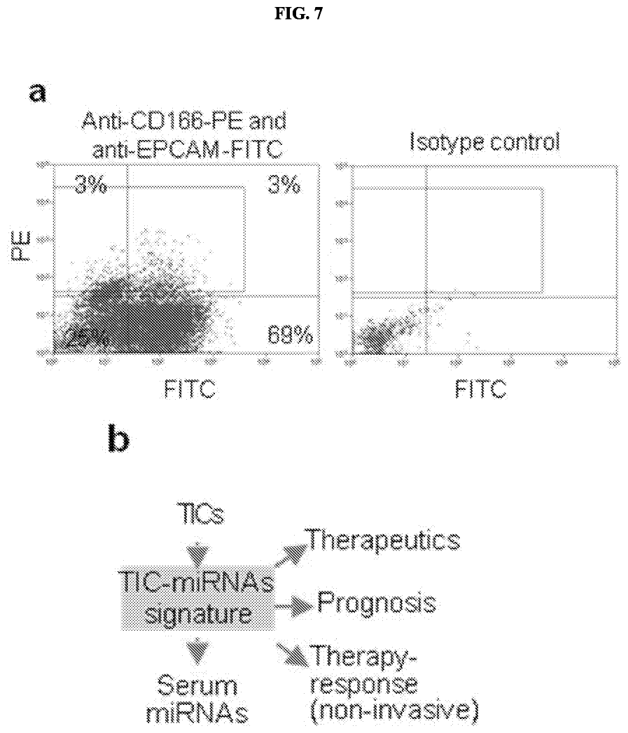

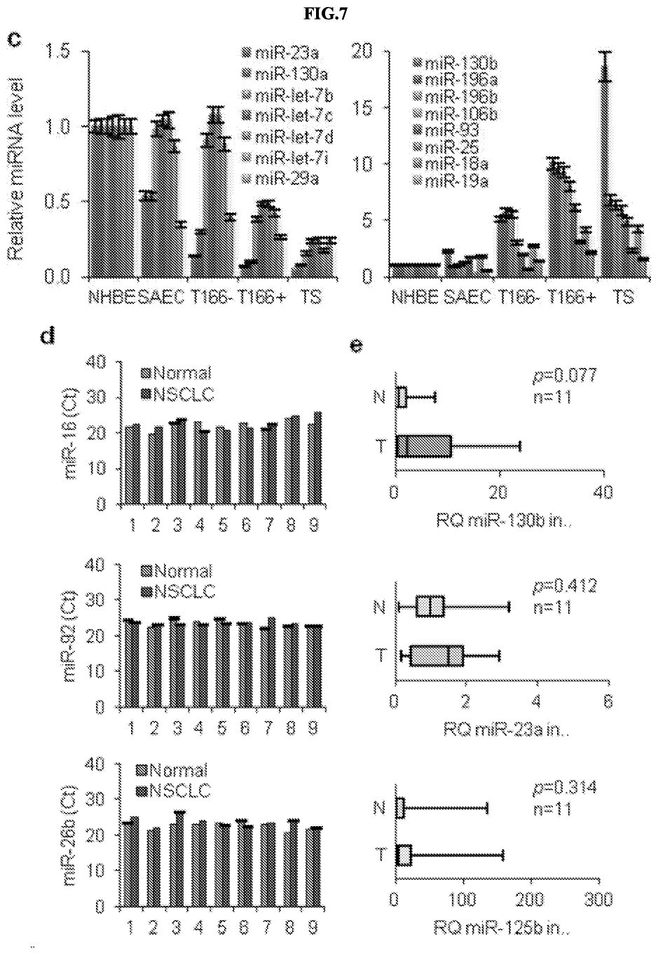

To uncover miRNAs which are major regulators of lung TICs, this study confirmed that patient-derived tumourspheres and CD166⁺ tumour cells were tumorigenic even when transplanted subcutaneously into mice at low cell numbers, whereas CD166⁻ tumour cells and two normal human primary lung epithelial cell lines (NHBE, human bronchial epithelial cells; and small airway epithelial cells (SAEC)) were completely devoid of this ability (FIG. 1a). To exclude non-tumour cells, the study sorted for cells that were EPCAM⁺ carcinoma cells (FIG. 7a). Limiting dilution cell transplantation analysis for tumour initiation showed that as few as 500 CD166⁺ cells could be serially propagated in immunodeficient NOD.Cg-Prkdcscid Il2rgtm1Wjl/SzJ (NSG) mice, whereas 100,000 CD166⁻ cells had no ability to form tumours in serial transplantation assays performed subcutaneously (FIG. 1b). These data confirmed both tumourspheres and CD166⁺ tumour cells to be enriched for TICs. To profile the miRNA expression from lung TICs and their differentiated progenies, which are non-tumour initiating, miRNA microarray was performed (FIG. 7b). The study first identified miRNAs which were enriched in CD166+ TICs relative to CD166− non-TICs (n=3), and intersected these with miRNAs enriched in patient-derived tumourspheres relative to normal NHBE and SAEC. This method, utilizing two distinct manners of purifying for TICs, enabled us to robustly identify a conserved set of miRNAs which were exclusive to TICs but not non-TICs (FIG. 1 and Table 1).

TABLE 1
Top-most upregulated and downregulated miRNAs in tumor-
initiating cells in non-small cell lung cancer.

| | log₂ (TS/(NHBE/ | log₂ (T166+/ | |
|---|---|---|---|
| | SAEC)) | T166−) | |
| | (FC, cut-off >2 | (FC, cut-off >1.5 | |
| miRNA | or <−2) | or <−1.5) | Difference |
| miR-1290 | 4.5 | 0.7 | up |
| miR-130b | 3.6 | 1.0 | (n = 19) |
| miR-1246 | 3.6 | 2.4 | |
| miR-630 | 3.1 | 1.1 | |
| miR-196a | 2.8 | 0.7 | |
| miR-196b | 2.6 | 0.7 | |
| miR-9* | 2.4 | 0.7 | |
| miR-106b | 2.4 | 1.4 | |
| miR-93 | 1.9 | 1.6 | |
| miR-25 | 1.9 | 2.2 | |
| miR-7 | 1.8 | 0.9 | |
| miR-18a | 1.7 | 0.6 | |
| miR-192 | 1.6 | 0.6 | |
| miR-320c | 1.4 | 0.6 | |
| miR-19a | 1.1 | 0.6 | |
| miR-3667-5p | 1.1 | 0.7 | |
| miR-483-5p | 1.0 | 0.6 | |
| miR-374a | 1.0 | 0.7 | |
| miR-96 | 1.0 | 0.6 | |
| miR-377 | −1.1 | −0.9 | down |
| miR-29a | −1.3 | −0.6 | (n = 14) |
| miR-376a | −1.4 | −0.9 | |
| miR-1260 | −1.4 | −1.0 | |
| miR-99a | −1.5 | −0.7 | |
| miR-1274a | −1.6 | −0.6 | |
| miR-125b | −1.8 | −1.3 | |
| miR-513a-5p | −1.8 | −0.6 | |
| let-7d | −2.1 | −1.1 | |
| let-7c | −2.4 | −1.7 | |
| let-7i | −2.4 | −1.1 | |
| let-7b | −2.7 | −1.2 | |
| miR-130a | −3.3 | −1.6 | |
| miR-23a | −3.8 | −1.0 | |

Note:
TS, tumor sphere; FC, fold change; T, lung cancer patient-derived tumor cells; 166+, CD166+; 166−, CD166−.

The top downregulated lung TIC-associated miRNAs include miR-23a, miR-130a, let-7 family, miR-513a-5p, miR-125b and miR-29a, whereas the top upregulated miRNAs include miR-1290, miR-130b, miR-1246, miR-630, miR-196a/b, miR-9/9* and miR-17˜92 cluster and its miR-106b-25 analogues. Reduced let-7 miRNA family expression, which is associated with significantly shorter cancer patient survival, was found in TICs. Similarly, miR-23a and miR-130a were shown to be downregulated in chronic myeloid leukaemia, and miR-29a/b/c was frequently reduced in a variety of cancers that include lung cancer. Conversely, upregulation of miR-17˜92 cluster and its paralogues miR-106b-25, which were elevated in lung TICs, was found in several other cancers, as these miRNAs promoted the rapid proliferation and undifferentiated phenotype of lung epithelial progenitor cells, as well as playing a role in embryonic lung development. Other miRNAs that include miR-130b, miR-196a/b and miR-9/9*, similarly, were found to contribute towards the progression of other cancer types.

From the compendium of candidate miRNAs, which were validated by quantitative RT-PCR (qRT-PCR; FIG. 7c), at least several of these miRNAs have not been previously implicated in TIC function and oncogenesis. This study chose to focus on miR-1246 and miR-1290 because they represent the topmost upregulated miRNAs among the TIC miRNA signature, and could be important drivers for cancer progression. Using another independent lung cancer patient cohort for which tumourspheres were generated, and also purified for CD166⁺ cells, this study indeed confirmed miR-1246 and miR-1290 expression to be elevated more than fivefold in CD166⁺ TICs, and increased 6 and 30 times, respectively, in the corresponding patient-derived tumourspheres when compared with either normal lung epithelial cells or CD166⁻ non-TICs (FIG. 1d).

Because the initial evidence suggested that miR-1246 and miR-1290 could be restricted to lung TICs, this study first sought to examine their expression patterns within human lung tumours as this serves to provide a clinically relevant context for studying their function. To broadly demonstrate the expression and specificity of certain miRNAs to NSCLC carcinoma cells, this study analysed their levels in matched lung tumours and adjacent non-neoplastic tissues of the same individuals (n=11 pairs) by qRT-PCR using miR-16, miR-92 and miR-26b as endogenous controls that remained unaltered between tumour and normal tissues (FIG. 7d). Both miR-1246 and miR-1290 showed consistent upregulation in tumours compared with their adjacent non-neoplastic tissues (P<0.01 for miR-1246, P<0.01 for miR-1290 by unpaired t-test; FIG. 1e). More importantly, the comparative expression of other miRNA candidates such as miR-130b, miR-23a and miR-125b, which were initially found to be also enriched in TICs, did not provide strong evidence that they were restricted to tumours, whereas miR-1246 and miR-1290 did (FIG. 7e). Similarly, by analysing the miRNA sequencing-based expression of a far larger cohort of paired tumour and adjacent tissues deposited in The Cancer Genome Atlas (TCGA), this study observed a highly significant elevation of miR-1246 in tumour samples, relative to adjacent tissues (FIG. 1f and Table 2).

TABLE 2
Expression of miR-1246 and miR-1290 across different
human cancers based on TCGA miRNA-Seq data.

| | | miR-1246 (N/T) | | miR-1290 (N/T) | | No. |
|---|---|---|---|---|---|---|
| Cancer | Description | FC | p-value | FC | p-value | patients |
| LUSC | Lung squamous cell carcinoma | 0.574496 | 2.71E−08 | 0.080643 | 0.012091 | 504 |
| STAD | Stomach adenocarcinoma | 0.114311 | 0.00196 | 0.016124 | 0.198385 | 443 |
| UCEC | Uterine corpus endometrial Carcinoma | 0.250787 | 0.002741 | 0.041955 | 0.083053 | 548 |
| LUAD | Lung adenocarcinoma | 0.107022 | 0.004347 | 0.022732 | 0.171509 | 521 |
| HNSC | Head and neck squamous cell carcinoma | 0.15125 | 0.021645 | −0.01652 | 0.453623 | 528 |
| PRAD | Prostate adenocarcinoma | −0.0091 | 0.021979 | −0.00662 | 0.391253 | 498 |
| COAD | Colon adenocarcinoma | 0.343819 | 0.024155 | 0.053833 | 0.163521 | 461 |
| READ | Rectum adenocarcinoma | 0.559734 | 0.026974 | 0.038959 | 0.490673 | 171 |
| BLCA | Bladder urothelial carcinoma | 0.08001 | 0.071999 | 0.017168 | 0.319218 | 412 |
| LIHC | Liver hepatocellular carcinoma | 0.018394 | 0.083837 | 0.009056 | 0.265112 | 377 |
| BRCA | Breast invasive carcinoma | 0.030557 | 0.121647 | 0.001774 | 0.641866 | 1098 |
| ESCA | Esophageal carcinoma | 0.044802 | 0.135981 | 0.014069 | 0.483665 | 185 |
| THCA | Thyroid carcinoma | −0.00101 | 0.203217 | −0.01344 | 1.61E−05 | 507 |
| CESC | Cervical squamous cell carcinoma and | 0.164581 | 0.26667 | 0.025542 | 0.613878 | 308 |
| | endocervical adenocarcinoma | | | | | |
| SKCM | Skin cutaneous melanoma | 0.561717 | 0.347114 | 0.11688 | 0.546408 | 470 |
| KIRC | Kidney renal clear cell carcinoma | 0.006704 | 0.477279 | 0.001932 | 0.617102 | 536 |
| RICH | Kidney chromophobe | 0.003051 | 0.554628 | 0 | n.a. | 66 |
| THYM | Thymoma | 0.046651 | 0.629116 | 0.022467 | 0.729072 | 124 |
| CHOL | Cholangiocarcinoma | 0.004948 | 0.656721 | 0 | NA | 36 |
| PAAD | Pancreatic adenocarcinoma | 0.011378 | 0.676258 | 0.003797 | 0.852846 | 185 |
| PCPG | Pheochromocytoma and paraganglioma | 0.002318 | 0.843321 | 0 | n.a. | 179 |

Note:
FC, fold change; NA, not applicable; N/T, normal versus tumor. TCGA, The Cancer Genome Atlas.

The study next assessed whether the expression of miR-1246 and miR-1290 might be heterogeneous among the tumours of different NSCLC patients, and the implications for disease outcome. By in situ-hybridization (ISH) assay on tissue microarrays from a cohort of 143 patients (n=197 tumour cores; FIG. 1g), miR-1246 expression negatively correlated with patient survival (P=0.016 by log-rank test), while miR-1290 had a weaker correlation (P=0.082 by log-rank test; FIG. 1h and Tables 3 and 4). Patients bearing tumours with higher miR-1246 expression, as assessed by staining intensity, showed elevated subdistribution hazard ratio compared with those harbouring tumours with lower miR-1246 expression (2.8, 95% confidence interval 1.22-6.66) (FIG. 1h). The study further sought to verify the above findings with another cohort of lung cancer patients, this time analysing the miRNA-sequencing data set from TCGA. Consistently, higher miR-1246 or miR-1290 expression in tumours was associated with shorter patient survival periods (P=0.007 and P=0.024 by log-rank test, respectively; FIG. 7f,g).

TABLE 3
Clinical pathologic characteristics of 143 study subjects
based on the expression intensity of miR-1246.

| | Total | | Low | | High | | |
|---|---|---|---|---|---|---|---|
| Expression of miR-1246 | (n = 143) | | (n = 30) | | (n = 113) | | p-value |
| Mean age (yrs) | 63.6 | (10.0) | 65.5 | (7.6) | 63.1 | (10.5) | 0.249 |
| Gender (%) | | | | | | | |
| Male | 97 | (68) | 23 | (77) | 74 | (65) | 0.279 |
| Female | 46 | (32) | 7 | (23) | 39 | (35) | |
| Histology (%) | | | | | | | |
| Adenocarcinoma | 97 | (68) | 11 | (37) | 87 | (76) | 0.001 |
| Squamous cell carcinoma | 46 | (32) | 19 | (63) | 27 | (24) | |
| Mean tumor size (cm) | 3.79 | (2.02) | 4.29 | (2.22) | 3.66 | (1.95) | 0.141 |
| T stage (%) | | | | | | | |
| T1 | 33 | (24) | 9 | (32) | 24 | (22) | 0.031 |
| T2 | 71 | (52) | 18 | (64) | 53 | (49) | |
| T3 | 16 | (12) | 0 | (0) | 16 | (15) | |
| T4 | 16 | (12) | 1 | (4) | 15 | (14) | |
| N stage (%) | | | | | | | |
| N0 | 104 | (76) | 27 | (93) | 77 | (72) | 0.024 |
| N1-3 | 32 | (24) | 2 | (7) | 30 | (28) | |
| M stage (%) | | | | | | | |
| M0 | 132 | (97) | 29 | (100) | 103 | (96) | 0.578 |
| M1 | 4 | (3) | 0 | (0) | 4 | (4) | |
| AJCC stage (%) | | | | | | | |
| I | 81 | (61) | 27 | (96) | 54 | (51) | <0.001 |
| II | 24 | (18) | 0 | (0) | 24 | (23) | |
| III | 23 | (17) | 1 | (4) | 22 | (21) | |
| IV | 5 | (4) | 0 | (0) | 5 | (5) | |
| Grade (%) | | | | | | | |
| Well-differentiated | 9 | (6) | 3 | (10) | 6 | (5) | 0.522 |
| Moderately differentiated | 74 | (53) | 13 | (43) | 61 | (55) | |
| Poorly differentiated | 46 | (33) | 12 | (40) | 34 | (31) | |
| Undifferentiated | 11 | (8) | 2 | (7) | 9 | (8) | |
| Status (%) | | | | | | | |
| Alive | 66 | (46) | 18 | (60) | 48 | (42) | 0.022 |
| Lung cancer related death | 63 | (44) | 7 | (23) | 56 | (50) | |
| Non-lung cancer related death | 14 | (10) | 5 | (17) | 9 | (8) | |

TABLE 4
Clinical pathologic characteristics of 143 study subjects
based on the expression intensity of miR-1290.

| | Low | | High | | |
|---|---|---|---|---|---|
| Expression of miR-1290 | (n = 33) | | (n = 110) | | p-value |
| Mean age (yrs) | 64.6 | (7.7) | 63.3 | (10.6) | 0.523 |
| Gender (%) | | | | | |
| Male | 23 | (70) | 74 | (67) | 0.835 |
| Female | 10 | (30) | 36 | (33) | |
| Histology (%) | | | | | |
| Adenocarcinoma | 17 | (52) | 80 | (73) | 0.033 |
| Squamous cell carcinoma | 16 | (48) | 30 | (27) | |
| Mean tumor size (cm) | 3.27 | (1.73) | 3.95 | (2.08) | 0.101 |
| T stage (%) | | | | | |
| T1 | 12 | (39) | 21 | (20) | 0.001 |
| T2 | 19 | (61) | 52 | (50) | |
| T3 | 0 | (0) | 16 | (15) | |
| T4 | 0 | (0) | 16 | (15) | |
| N stage (%) | | | | | |
| N0 | 31 | (97) | 73 | (70) | 0.001 |
| N1-3 | 1 | (3) | 31 | (30) | |
| M stage (%) | | | | | |
| M0 | 32 | (100) | 100 | (96) | 0.573 |
| M1 | 0 | (0) | 4 | (4) | |
| AJCC stage (%) | | | | | |
| I | 31 | (100) | 50 | (49) | <0.001 |
| II | 0 | (0) | 24 | (24) | |
| III | 0 | (0) | 23 | (23) | |
| IV | 0 | (0) | 5 | (5) | |
| Grade (%) | | | | | |
| Well-differentiated | 4 | (12) | 5 | (5) | 0.453 |
| Moderately differentiated | 16 | (48) | 58 | (54) | |
| Poorly differentiated | 10 | (30) | 36 | (34) | |
| Undifferentiated | 3 | (9) | 8 | (7) | |
| Status (%) | | | | | |
| Alive | 17 | (52) | 49 | (45) | 0.567 |
| Lung cancer related death | 12 | (36) | 51 | (46) | |
| Non-lung cancer related death | 4 | (12) | 10 | (9) | |

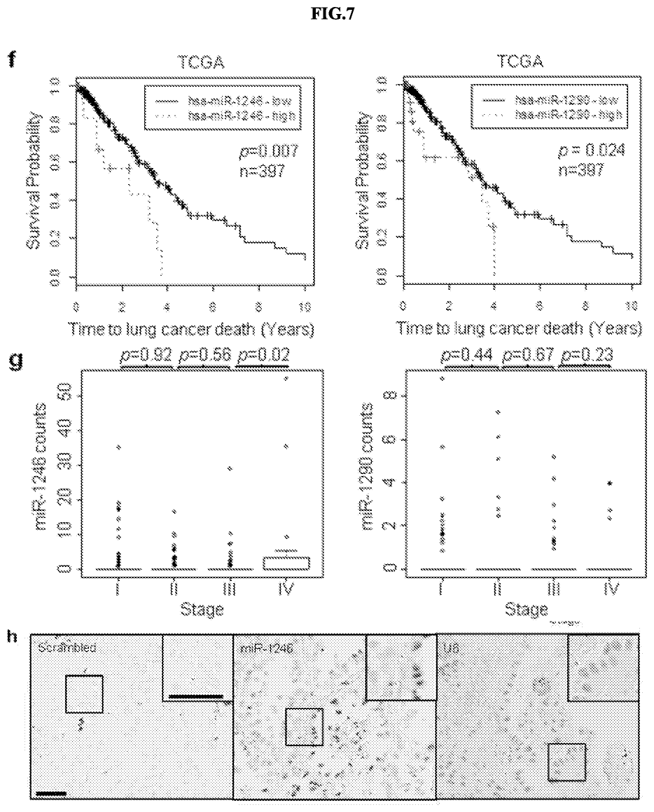

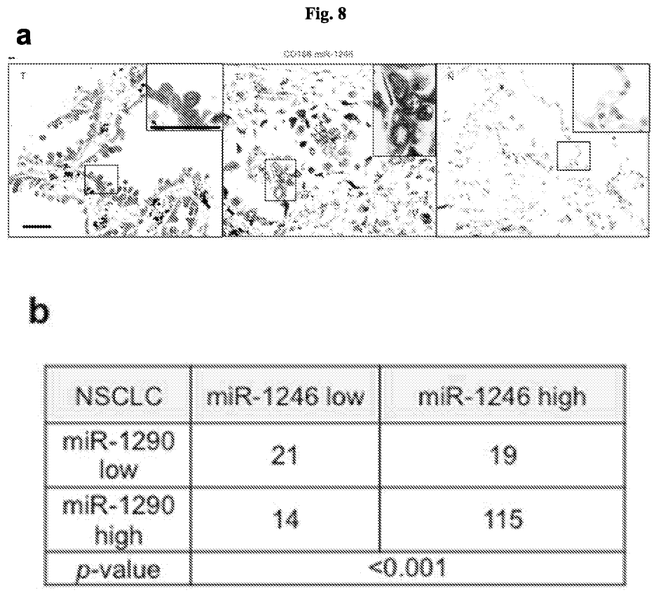

Since miR-1246 was initially found to be enriched in flow cytometry purified CD166 cells, we proceeded to verify whether the miRNA was also found within TICs present in patient tumour sections. To compare the cellular expression of miR-1246 and CD166 protein, ISH and immunohistochemistry were first performed separately on serial sections of both malignant and normal lung tissues. Mir-1246 was strongly localized to the cytoplasm and nucleus in tumour cells, while remained weak or undetectable in most of the normal lung epithelial cells (FIG. 7h). Similarly, CD166 positivity was predominately detected in the cytoplasm and cell membrane in tumour cells and almost absent in normal lung tissues (FIG. 1i and Table 5). To directly correlate the expression pattern of miR-1246 with CD166, ISH and immunohistochemistry were combined on the same tissue or tumour section. MiR-1246 expression was predominantly restricted to CD166⁺ cells in primary NSCLC tumours, and largely absent in CD166⁻ tumour cells and normal lung epithelial cells (FIG. 8a). χ²-Analysis in NSCLC tissues showed a strong correlation between the intensity of miR-1246 expression and miR-1290 expression by ISH (P<0.001 by Student's t-test; FIG. 8b).

TABLE 5
Association between CD166 expression
and miR-1246 or miR-1290 expression.

| | CD166 (0~2+) | CD166 (3+) | p-value |
|---|---|---|---|
| miR-1246 (0~2+) | 154 | 19 | 0.004 |
| miR-1246 (3+) | 14 | 8 | |
| miR-1290 (0~2+) | 123 | 18 | 0.022 |
| miR-1290 (3+) | 39 | 15 | |

MiR-1246 and miR-1290 Confer Tumorigenicity.

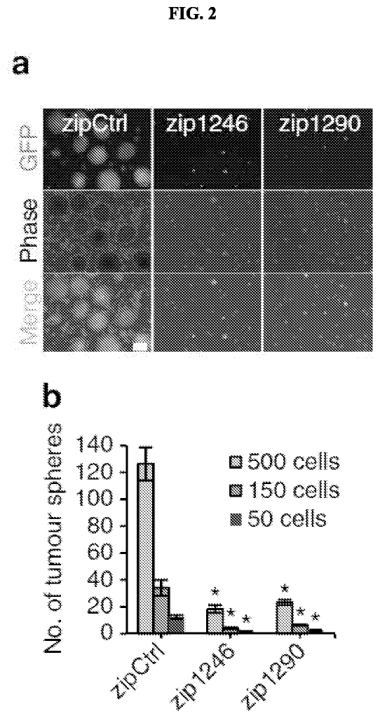

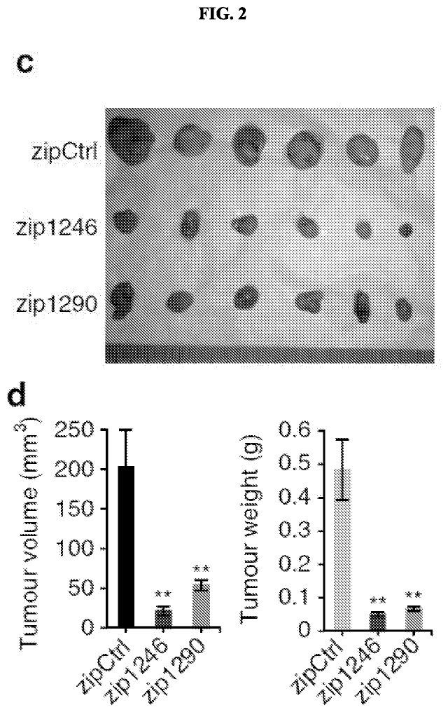

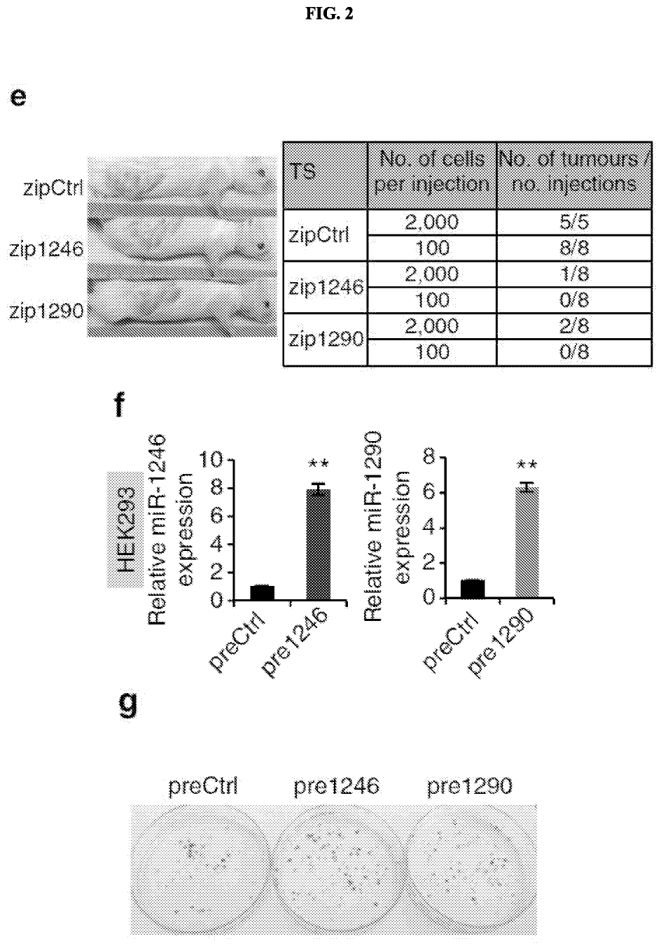

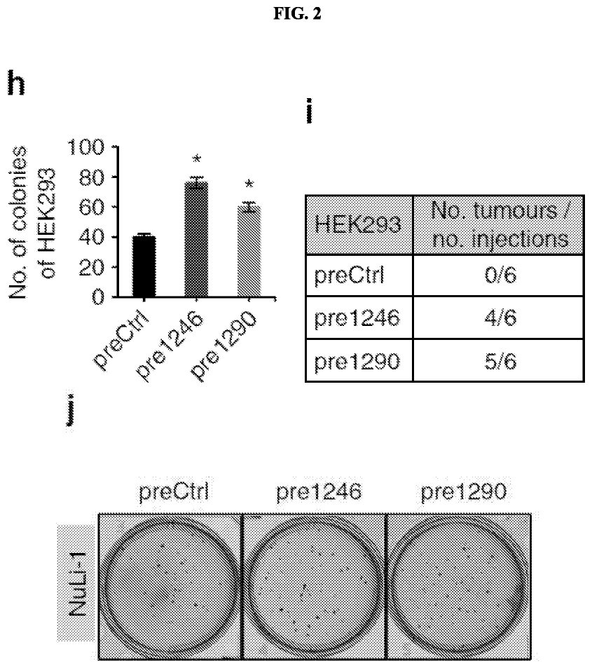

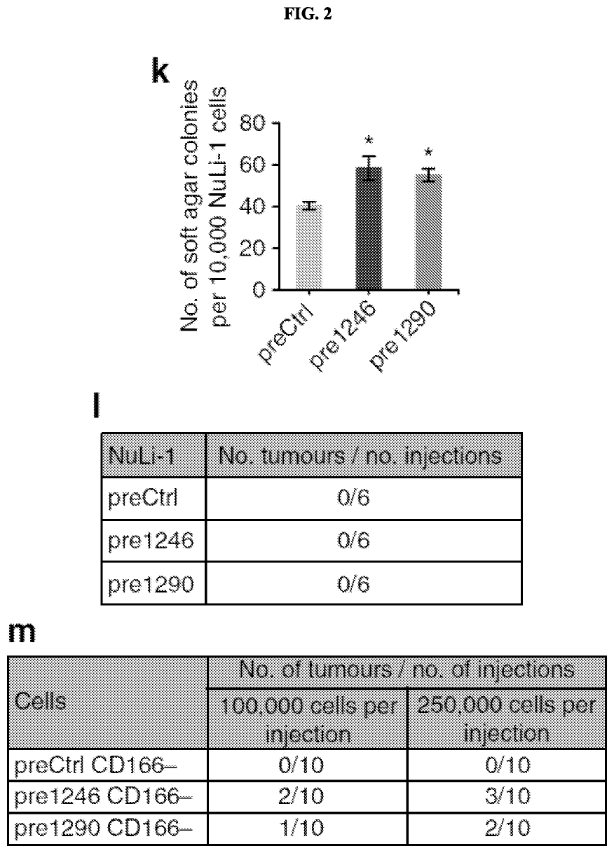

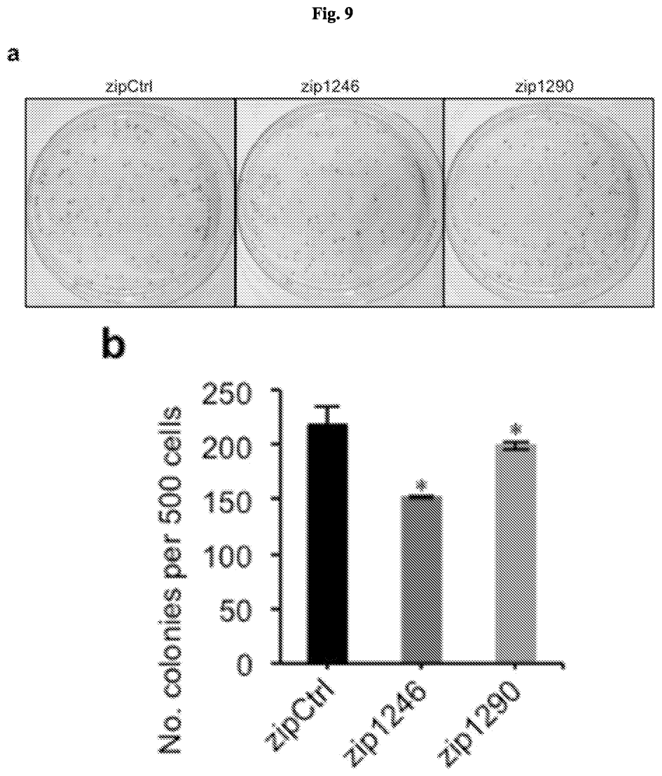

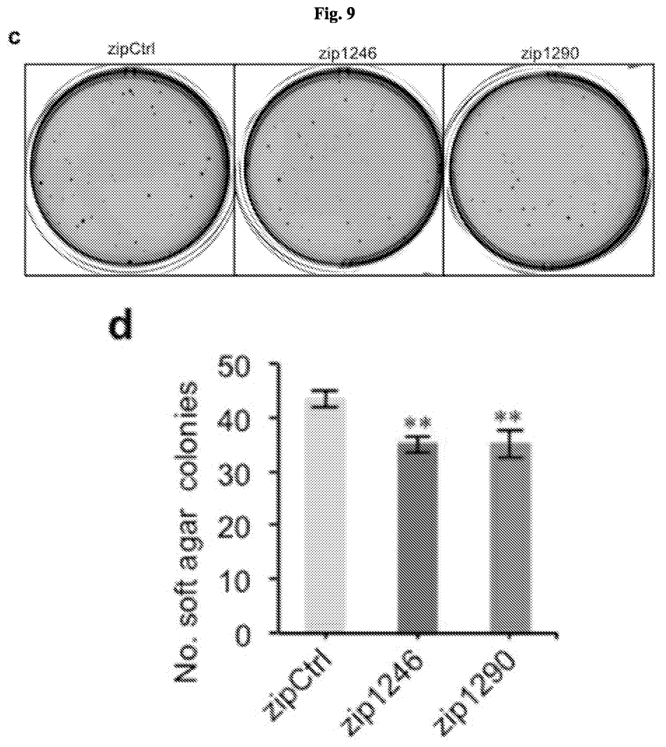

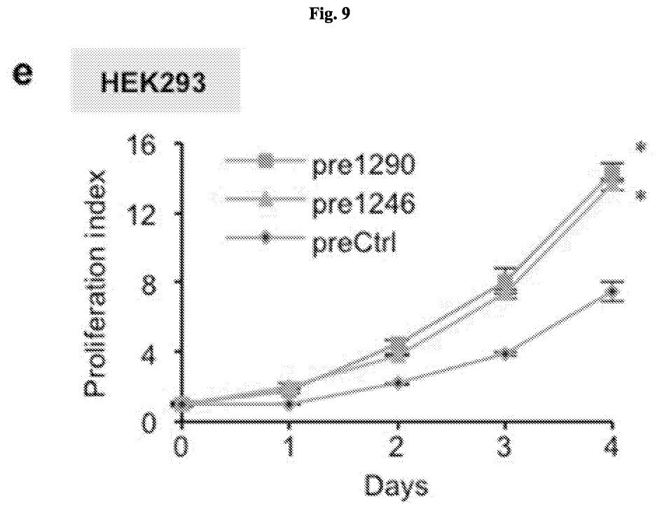

The highly enriched expression of miR-1246 and miR-1290 in lung CD166⁺ TICs, but not in CD166⁻ cells and normal lung epithelial cells, strongly suggests these two miRNAs to be crucial for tumour initiation and establishment. To test this, the study utilized highly specific miRZip lentiviral anti-miR-1246 and anti-miR-1290 to knockdown miR-1246 and miR-1290 in lung tumourspheres and assessed their tumorigenic potential in cell cultures and in mice. Dissociated tumourspheres were initially plated on either soft agar or 2D adherent cell cultures-assays which select for the growth of bulk cancer cells, including differentiated cancer cell populations. Interestingly, this resulted only in a small decrease in colony numbers, indicating that the loss of miR-1246 and miR-1290 did not impact cell growth and proliferation under these cell culture conditions (FIG. 9a-d). However, when tumourspheres bearing either miR-1246 or miR-1290 knockdown were subjected to extended periods of culture in serum-free sphere-forming condition that selects for TICs and stem cells, their numbers were markedly reduced by at least 5.4-fold (FIG. 2a). In a limiting dilution assay, sphere-formation efficiency was consistently reduced when 500, 150 and 50 cells were plated (FIG. 2b). When tumourspheres were transplanted into immune-compromised mice, those bearing knockdown of either miR-1246 or miR-1290 alone markedly inhibited tumour growth (FIG. 2c,d). To confirm that the loss of miR-1246 or miR-1290 impacted tumour initiation, the study transplanted TS cells that were knocked down for either miRNA in limiting dilution cell numbers. While control-treated TS cells continued to form tumours with 100 cells, zip1246- or zip1290-treated cells were severely inhibited in their tumour initiation capability when 2,000 cells were transplanted, and were completely ablated of this ability with 100 cells, thus underscoring the role of these miRNAs in tumour initiation (FIG. 2e). Because lung TICs and cancer progression were dependent on miR-1246 or miR-1290, we assessed whether these miRNAs could confer tumorigenic potential to otherwise normal-like or nontumorigenic cells. Exogenous introduction of either miR-1246 or miR-1290 into immortalized human embryonic kidney cells (HEK293) modestly increased proliferation and colony formation in vitro (FIG. 2f-h and FIG. 9e), but surprisingly, was able to confer on these otherwise non-tumorigenic cells and their tumorigenic potential, as gauged by the ability of the miRNA-bearing cells to form tumours efficiently (FIG. 2i). In all, 66.7% (4/6) and 88.3% (5/6) of mice bearing miR-1246- and miR-1290-overexpressing HEK293 cells, respectively, grew tumours, whereas none of the mice transplanted with control treated cells formed tumours. To test the oncogenic potential of miRNAs in more physiologically relevant cell systems, the study introduced either miR-1246 or miR-1290 into immortalized human lung epithelial cells (NuLi-1). The overexpression of either miRNAs increased soft-agar colony formation in vitro, but could not initiate tumours even when 1×10⁶ cells were xenografted into NSG mice (FIG. 2j-l). This indicates that the miRNAs could drive neoplastic transformation of normal-like lung cells in vitro but did not confer full tumorigenic potential. More strikingly, overexpression of either miR-1246 or miR-1290 in non-tumorigenic CD166⁻ cancer cells was able to confer tumorigenic ability, as demonstrated by the formation of tumours when 100,000 cells were transplanted (FIG. 2m).

MiR-1246 and miR-1290 are Required for Lung Cancer Metastasis.

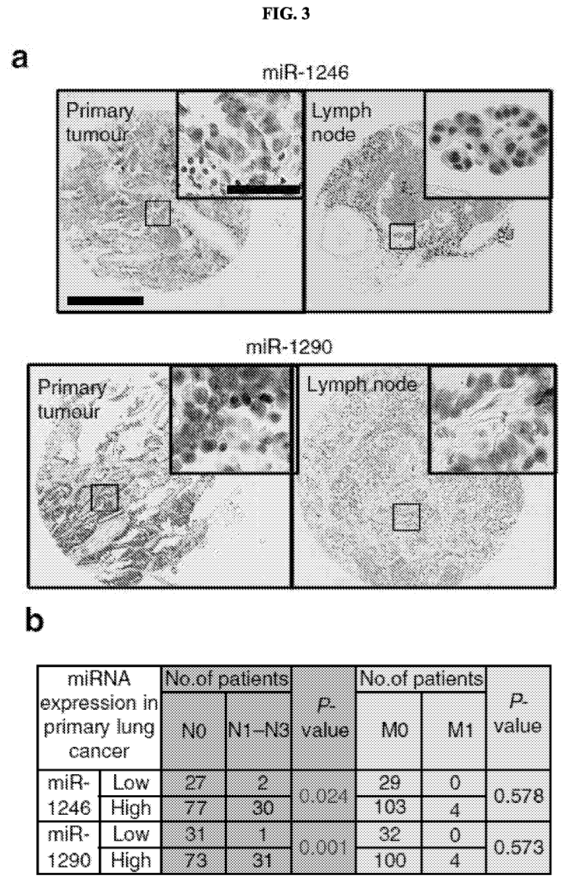

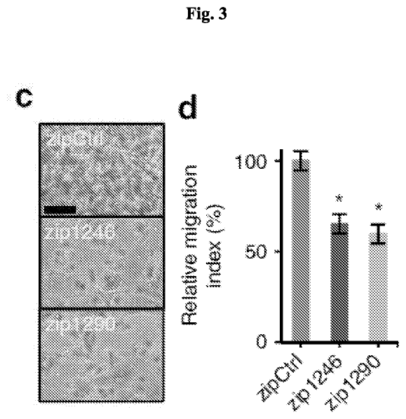

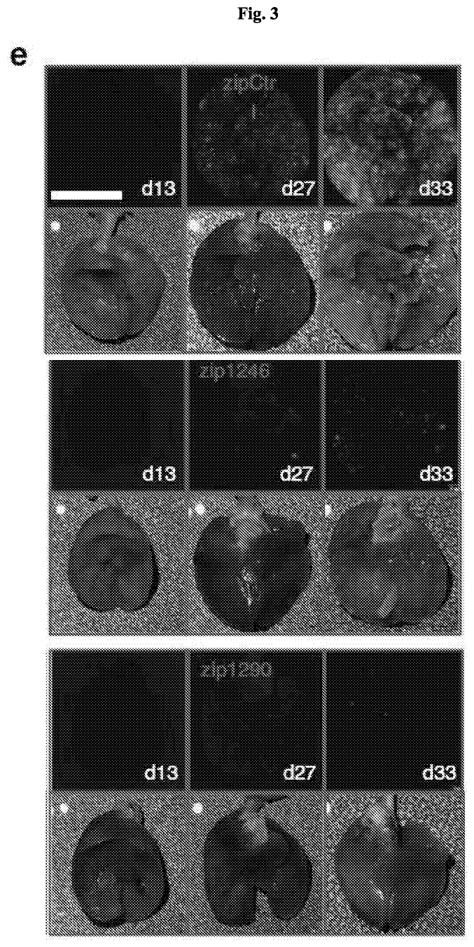

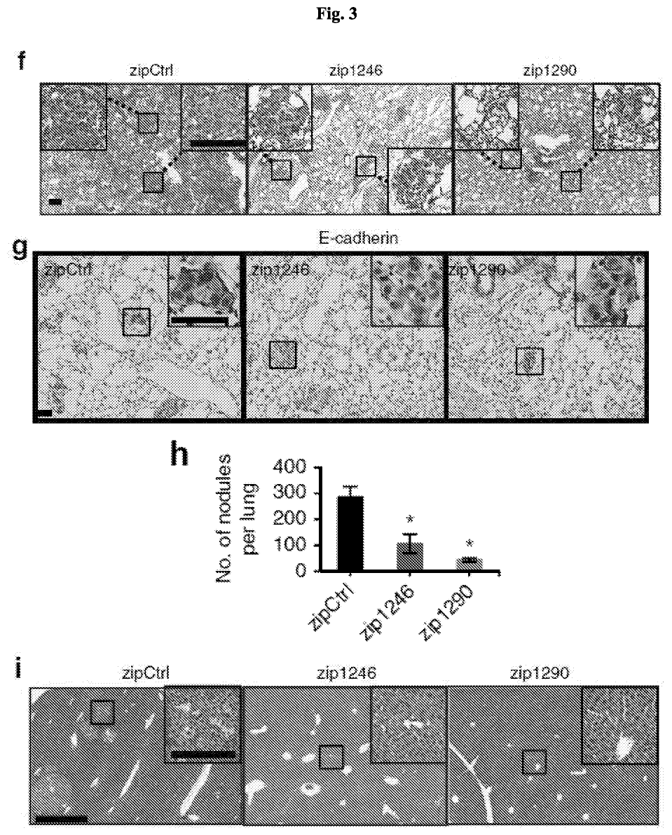

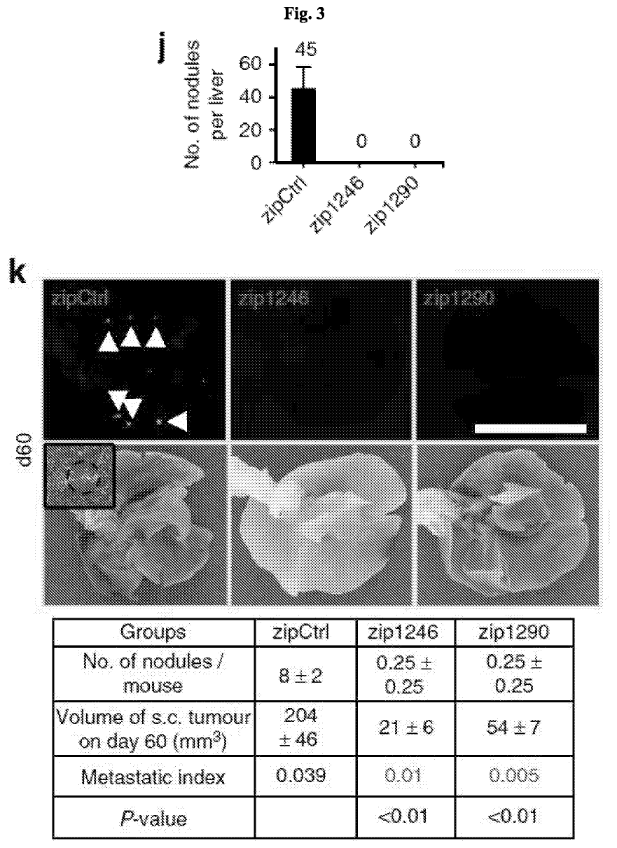

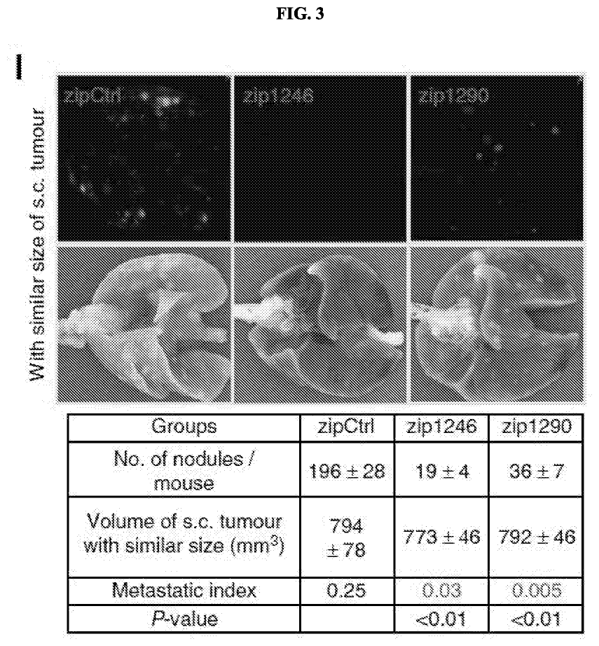

To determine whether miR-1246 and miR-1290 could confer metastatic traits to lung tumour cells, the study first assessed the expression of these miRNAs in cancer cells that metastasized to either the lymph node or distant organs. To determine the correlation between miRNA expression levels in primary tumours and lymph node metastasis, the study performed ISH for the miRNAs in paired primary lung tumours and corresponding lymph nodes (n=143 patients). Primary tumours that contain high miR-1246 or miR-1290 expression tended to correlate with the detection of metastatic tumour cells within lymph nodes, whereas those expressing low levels of the miRNAs did not (FIG. 3a,b). Hence, there was a strong association between expression of these miRNAs in primary tumour and lymph node metastasis (P=0.024 and P=0.001 by Student's t-test, respectively). Of note, however, this study did not observe a strong correlation for the miRNA expression levels between primary tumours and incidence of distant metastases; this is likely attributed to the very few cases of paired tumour and distant metastases we were able to obtain. Overall, the results suggest that miR-1246 and miR-1290 could contribute towards the metastatic abilities of lung tumour cells.

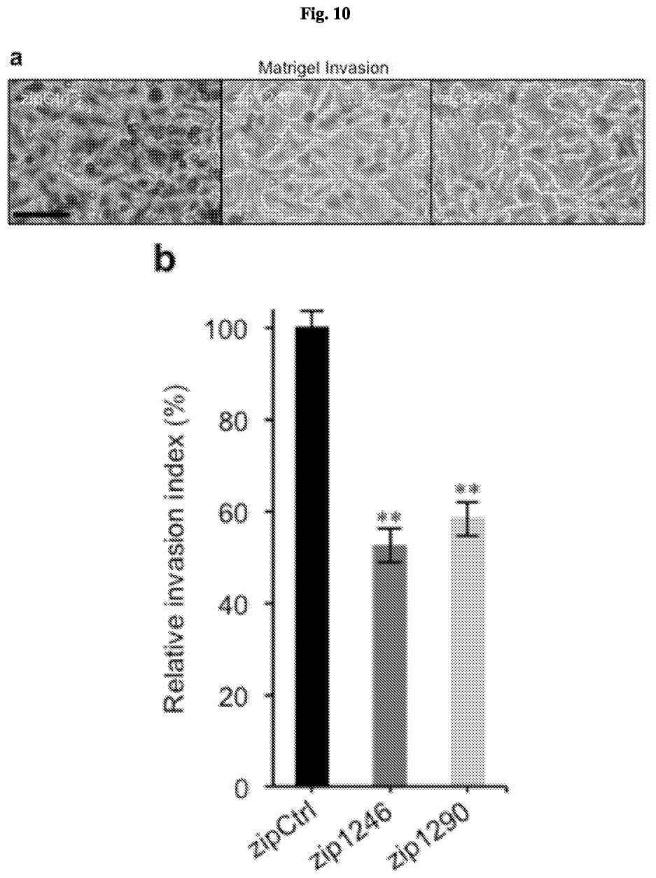

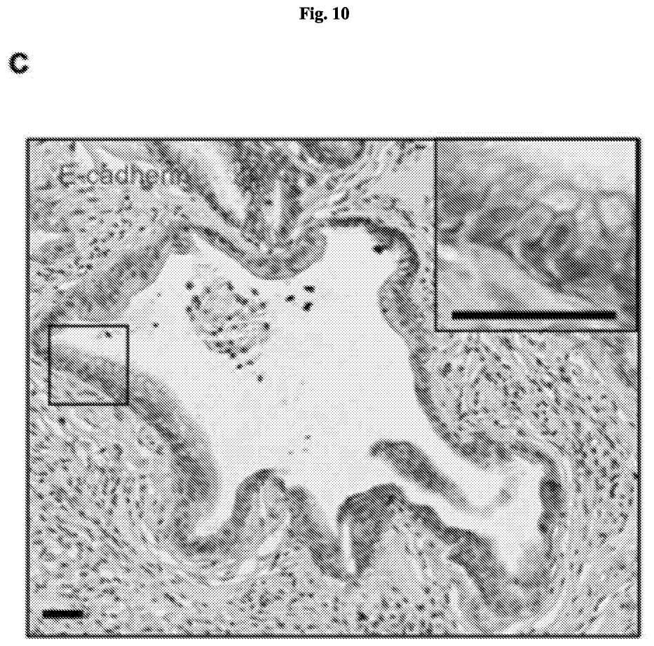

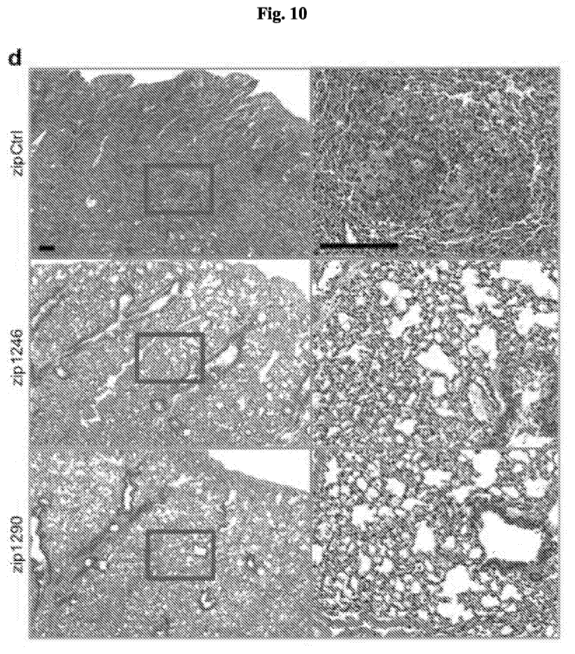

To test the migratory and invasive roles of these miRNAs in tumourspheres, the study first performed transmembrane migration and matrigel invasion assays for dissociated tumoursphere cells. Both the migration (FIG. 3c,d) and invasion capabilities (FIG. 10a,b) were markedly impaired on knockdown of either miR-1246 (zip1246) or miR-1290 (zip1290), thus suggesting that the miRNAs were necessary for the invasion, at least in vitro. The study further sought to examine whether miR-1246 and miR-1290 were indeed directly mediating the metastasis of lung TICs in animal models. The study initially performed experimental metastasis analyses by directly introducing the same number of tumoursphere cells containing zip1246, zip1290 or zip-control, into the lungs of immune-compromised NSG mice through tail-vein injection; this allowed the examination of their extravasation and colonization abilities—key stages of the metastatic cascade. After 5 weeks, metastatic nodules in the lungs and liver were markedly reduced on knockdown of either miR-1246 or miR-1290 in tumoursphere cells before tail-vein injection (FIG. 3e-j and FIG. 10c).

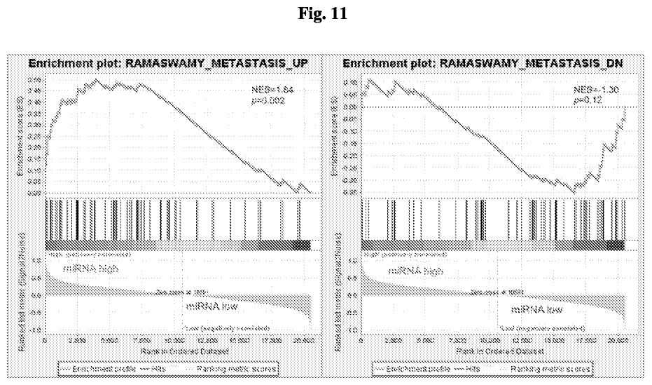

Subsequently, tumoursphere cells expressing either zip1246 or zip1290 were transplanted subcutaneously into NSG mice to determine their impact on spontaneous metastasis from primary tumours. Transplanting large numbers of tumoursphere cells (1_106), which contained either zip1246 or zip1290, gave rise to tumours that were reduced in size relative to control-treated cells. The latter cells seeded metastatic lung nodules efficiently 60 days post-transplantation, whereas miRNA-knockdown cells were deficient in this regard (FIG. 3k and FIG. 10d). However, owing to differences in size of primary tumours arising from treated and control cells, it could be likely that larger tumours are more capable of disseminating metastatic cells. To account for this difference, the metastatic index was calculated, which normalizes the number of metastatic nodules to the size of primary tumours. The metastatic index was indeed four to eight times lower in mice containing tumoursphere cells expressing either zip1246 or zip1290, relative to control-treated cells (FIG. 3k). Alternatively, the study allowed zip1246- and zip1290-expressing tumoursphere cells to form tumours, over a prolonged period of time, until they were approximately similar in size to control tumours (12 mm in diameter), and subsequent examined the number of metastatic lung nodules. The number of lung nodules in control groups was five to nine times more than those in groups containing either zip1246 or zip1290, given similar tumour burden (FIG. 3l). In human lung cancer patient cohorts, gene set enrichment analysis (GSEA) demonstrated a positive correlation between metastasis incidence and miR-1246 or miR-1290 expression in a variety of patient solid tumours that included lung adenocarcinoma (FIG. 11). Collectively, the data indicated that miR-1246 and miR-1290 play pivotal roles in mediating the spontaneous metastasis of primary tumour cells. This is consistent with the notion that highly aggressive tumours tend to contain an enriched population of TICs, which can augment tumour growth and metastasis.

Circulating miRNAs Levels Correlate with Therapy Response.

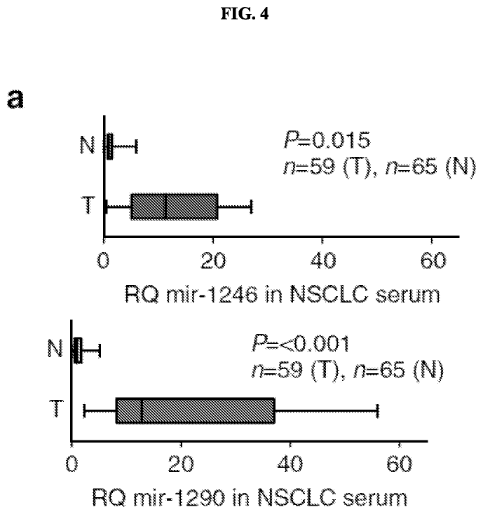

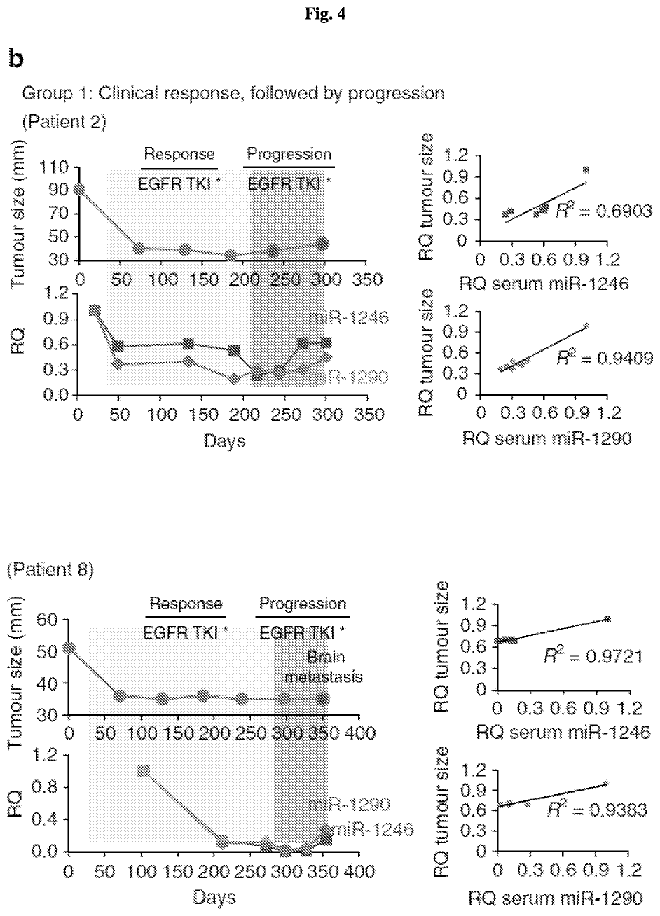

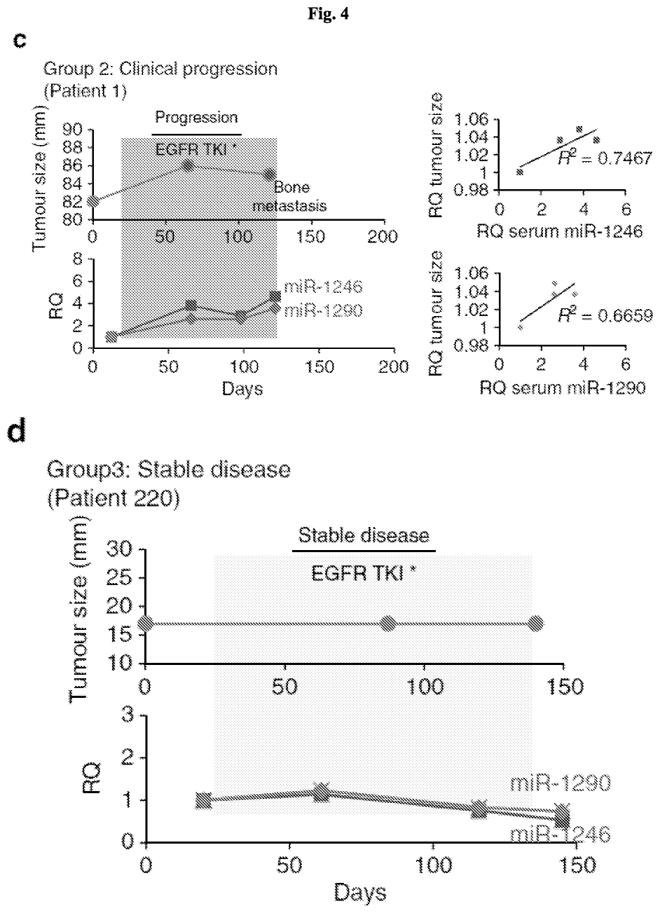

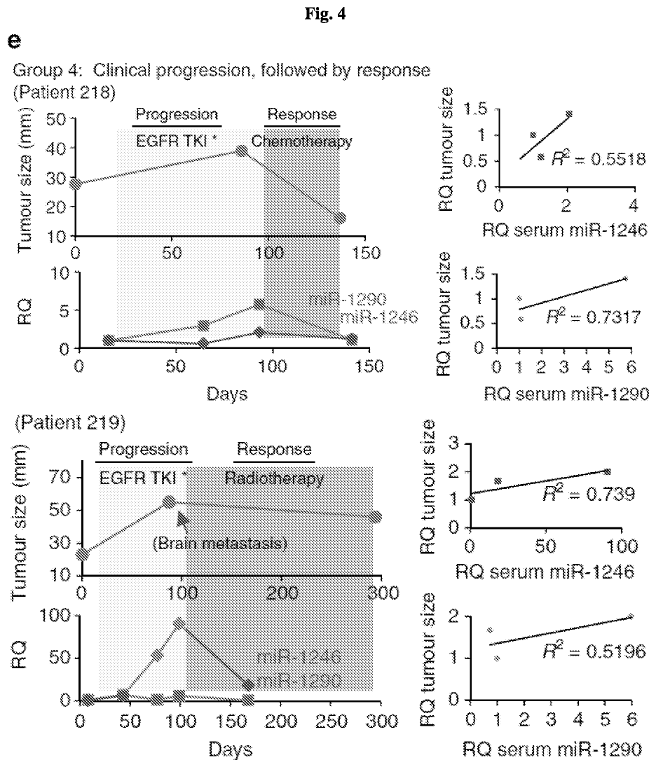

Circulating cell-free miRNAs have been reported in patients harbouring ovarian cancer, melanoma and lymphoma. The levels of certain miRNAs appear to be predictive of survival outcomes. In the vast majority of these studies, circulating miRNAs of different cancer patients and normal individuals are compared, typically at a single time point. Furthermore, in instances where circulating miRNA levels were correlated with therapy response, the measurements are obtained from different individuals, thereby confounding analyses. The direct contribution of miRNA levels to disease progression and therapy resistance remains unclear. Here, this study performed a longitudinal survey of circulating miR-1246 and miR-1290 in the same individuals to assess variation in their levels in response to ongoing EGFR tyrosine kinase inhibitor (TKI) treatment, which is a standard of care for NSCLC patients with tumours harbouring mutant EGFR. The study first examined the serum levels of miR-1246 and miR-1290 from NSCLC patients and healthy individuals (n=124) by qRT-PCR. MiR-1246 and miR-1290 levels increased 11.3 times and 12.8 times, respectively, in stage I-III NSCLC patients compared with healthy individuals, as expected (FIG. 4a).

To understand changes in the levels of miRNAs in response to therapy for the same individuals, the study recruited a small cohort of late-stage lung cancer patients who were assigned to receive EGFR TKI, and in some cases that progressed on EGFR TKI, with subsequent follow-up radiotherapy or chemotherapy. The baseline levels of circulating cell-free miRNA levels were ascertained before treatment and tracked at several time-points during the course of treatment. On recruitment, tumour sizes on computed tomography (CT) scan were determined using RECIST 1.1. In general, we were able to categorize patients into four subgroups, depending on the pattern of clinical response to treatment, based on either changes in the tumour size or the detection of metastatic disease. Group 1 (n=6) comprised of patients who initially responded to therapy, but later progressed as a result of tumour re-growth or occurrence of metastasis. Lung tumours in Patient 2 and 8, for instance, shrank rapidly by as much as 29-56% shortly following therapy, indicating that they were initially responders (FIG. 4b). During this response period, serum levels of miR-1246 and miR-1290 reduced by 69-87% and 63-90%, respectively. For these individuals, however, disease progressed; this is indicated by either tumour re-growth (Patient 2) or brain metastasis (Patient 8) despite continued EGFR TKI treatment. Concomitant increases in the serum miR-1246 and miR-1290 could be detected, thus suggesting their levels to be indicative of the patients' response to therapy. In Group 2 (n=3), patients did not respond to therapy from the onset, as determined by continued tumour growth or the detection of metastasis. In these individuals, levels of both miRNAs progressively increased during the course of the disease, resulting in treatment being withdrawn subsequently (FIG. 4c and Table 6). Group 3 consisted of a single individual (Patient 220), who had stable disease, and no change in the serum levels of miR-1246 or miR-1290 was detected (FIG. 4d).

TABLE 6
Tumor response or progression to clinical
therapy in NSCLC patients.

| | Clinical assessment of | | |
|---|---|---|---|
| | patient status byCT scan | | |
| SN | (response or progression) | Clinical therapy | Patient ID |
| 1 | Response followed by | Continuous EGFR | 2, 8, 120, |
| | progression (increase in | TKI | 124, 125, |
| | tumor burden or detection | | 137 |
| | of metastasis) | | |
| 2 | Progression (increase in | Continuous EGFR | 1, 105, |
| | tumor burden or detection | TKI | 215 |
| | of metastasis) | | |
| 3 | Stable disease (no significant | Continuous EGFR | 220 |
| | change in tumor burden and no | TKI | |
| | detectable metastasis) | | |
| 4 | Progression followed by | EGFR TKI, followed | 122, 128, |
| | response (decrease in tumor | by chemotherapy | 134, 218, |
| | burden or loss of metastasis) | or radiotherapy | 219 |

Note:
CT, computed tomography; EGFR, epidermal growth factor receptor; TKI, tyrosine kinase inhibitor.

In Group 4 (n=5), patients initially had disease progression but subsequently responded to therapy. All these patients received EGFR TKI as a first line of treatment, and either chemo- or radiotherapy as the second line of treatment. As an example, Patient 218 progressed on EGFR TKI as tumour grew by 41% on day 86; this was mirrored by increases in miR-1246 and miR-1290 levels. On switching to chemotherapy, the tumour shrank by 59% and the levels of both miRNAs were, similarly, reduced (FIG. 4e). Patient 219, similarly, showed marked tumour growth and occurrence of brain metastasis while on EGFR TKI, but later responded to whole-brain radiotherapy as measured by the reduction in tumour size (FIG. 4e). In this example, the levels of miR-1246 appeared to better mirror the response of patient to therapy than that of miR-1290. Of note, for the vast majority of patients in all four groups, serum levels of miR-1246 and miR-1290 showed a positive correlation with tumour size as well (FIG. 4b,c,e). Thus, the data suggest that serum miRNAs correlate well with the response of patients to ongoing therapy. This strategy of utilizing circulating miRNA levels as a surrogate for assessing disease progression status may, quite possibly, be more sensitive and predictive of metastasis that may not be readily detected in CT scans (FIG. 4b-e and Table 6). While CT scans remain diagnostically useful in most instances, they are of limited therapeutic benefit, hence, leading this study to explore if targeting miRNAs might be beneficial.

LNA-Targeting miRNAs Arrest PDX Tumour Growth.

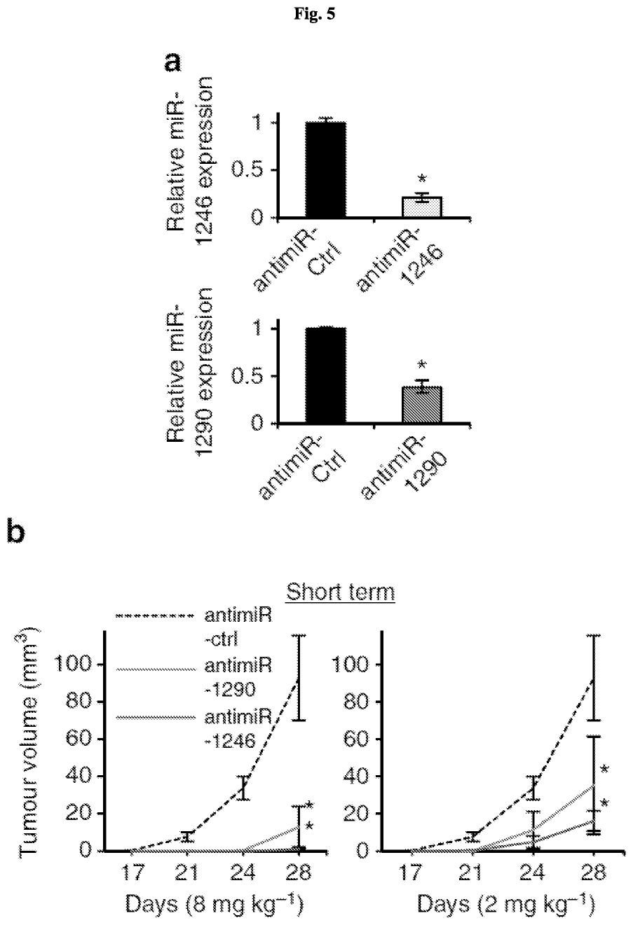

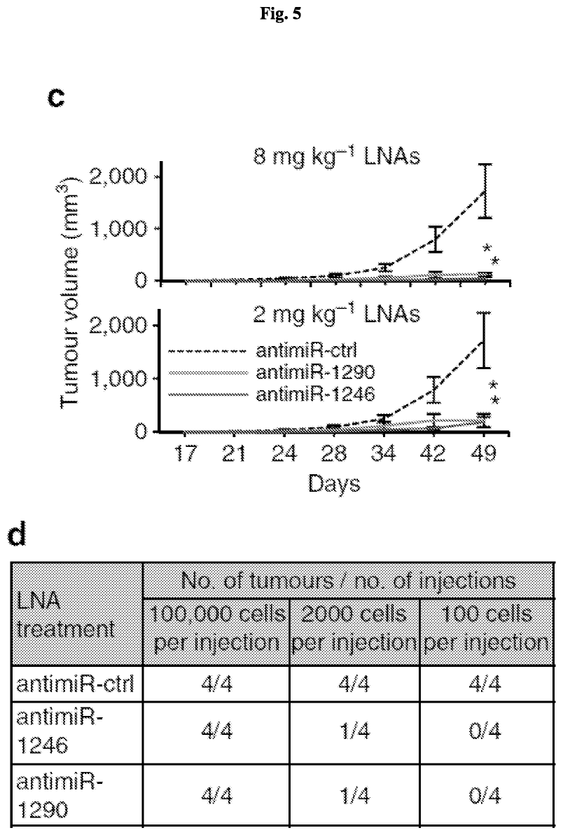

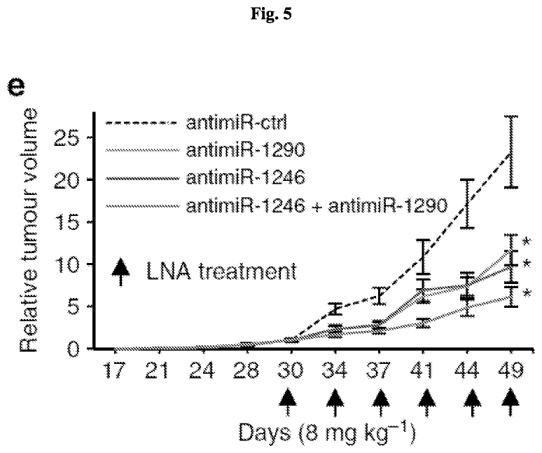

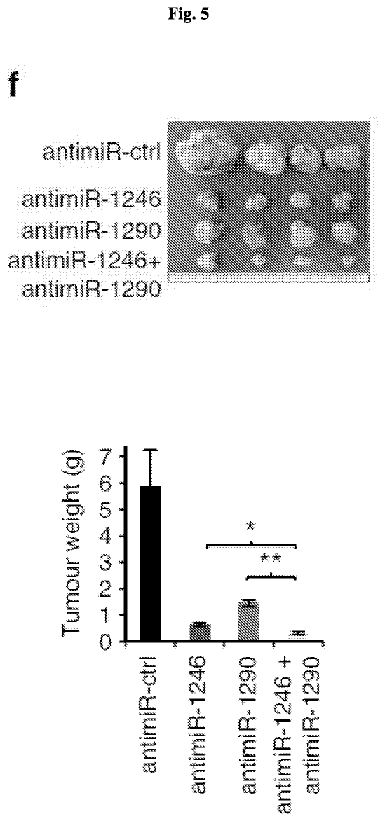

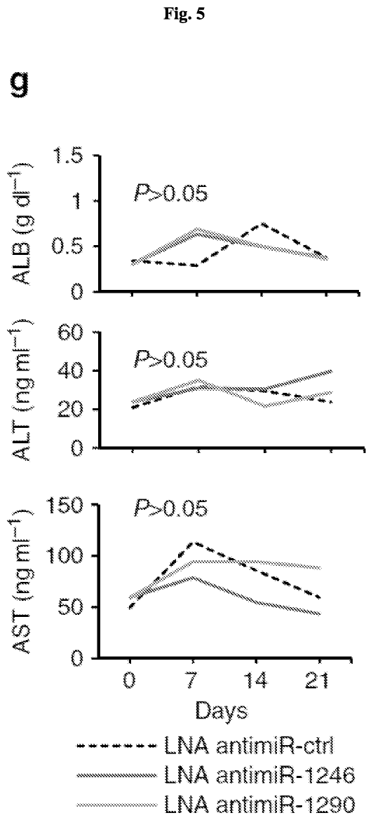

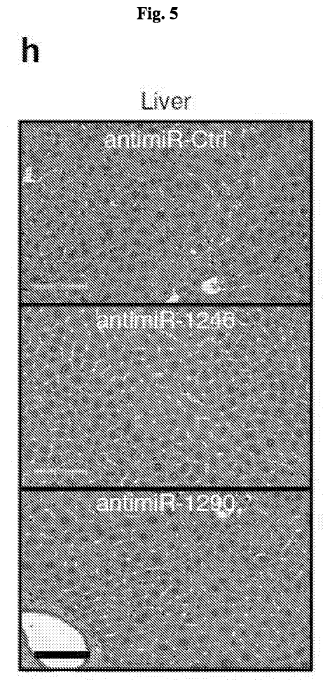

Because tumours appear to depend on miR-1246 and miR-1290 to progress, it was reasoned that the inhibition of these miRNAs might impact their growth. This study made use of LNA that can be administered into animals and silences specific miRNAs in a highly selective manner. This strategy has been experimentally tested in mice and non-human primates for the treatment of several diseases, and more recently, it has been applied in clinical trials for the treatment of hepatitis C. The utility of LNA against miR-1246 or miR-1290 was first evaluated in cell cultures by transfecting them into lung TICs expressing high levels of both miRNAs. Inhibition of either miR-1246 or miR-1290 significantly reduced their respective expression (FIG. 5a). To assess their therapeutic utility in a physiologically relevant manner, LNAs against either miR-1246 or miR-1290 were introduced intraperitoneally into NSG mice bearing patient-derived lung tumour xenografts. LNA (8 mg kg⁻¹) against either miR-1246 or miR-1290 was administered at the same time as lung TIC implantation (1×10⁵ cells). This not only delayed the onset of lung TIC-driven tumorigenesis, but also inhibited the long-term growth of xenograft tumours (FIG. 5b,c). Even when a lower dose of 2 mg kg was introduced, the delay of tumour initiation and reduction in growth was, similarly, observed (FIG. 5b,c). To directly assess the impact of LNAs on tumour initiation, we treated mice transplanted with limiting dilution number of TS cells. As expected, 100,000 xenografted cells continued to form tumours, albeit smaller and delayed, with LNA administration (8 mg kg⁻¹). The LNA, however, completely abrogated tumour-initiation when 100 cells were transplanted, thereby demonstrating the in vivo impact of blocking miR-1246 and miR-1290 on tumour initiation (FIG. 5d). To ascertain the impact of anti-miR-1246 and anti-miR-1290 LNA on pre-existing tumours, the study first allowed tumours to form (5 mm in length) before treating mice with 8 mg kg⁻¹ LNA at 3-4-day intervals. The outgrowth of tumours in the LNA-treated mice was largely inhibited, whereas control-treated mice continued to form large tumours, thereby indicating the therapeutic utility of silencing miR-1246 and miR-1290 in established tumours (FIG. 5e,f). Since the loss of each miRNA, on its own, was capable of reducing the tumorigenic ability of TS cells, the study tested if the combined inhibition of both miRNAs would demonstrate a more severe impact. Indeed, the concomitant administration of LNAs against miR-1246 and miR-1290 in mice bearing the pre-established tumours could result in a greater inhibition of tumour growth, and suggested the utility of targeting both miRNAs together (FIG. 5f).

A potential confounding challenge with therapeutic agents, including LNA, is animal toxicity, which can undermine their utility. To understand whether LNA targeting miR-1246 or miR-1290 would produce adverse side-effects in mice, the dynamic changes in albumin levels were profiled, as well as alanine aminotransferase (ALT) and aspartate aminotransferase (AST) activities, in mouse serum at different time points following LNA therapy at 8 mg kg⁻¹. The levels of albumin and activities of ALT and AST were, in fact, comparable to the sham-treated animals, thus indicating that the LNA, as a therapeutic agent, did not result in overt or measurable toxicity, at least, to the liver (FIG. 5g). This was further confirmed by the histological examination of liver sections obtained from the LNA-treated mice that did not show observable difference compared with control animals (FIG. 5h). Taken altogether, the results demonstrated that miR-1246 and miR-1290 contribute towards lung cancer progression through acting on TIC populations, and more importantly, provided a proof-of-concept principle that LNA approaches can be used to impact the behaviour of TICs by targeting miRNAs crucial for their function.

MT1G is a Target of miR-1246 and miR-1290 that Inhibit TICs.

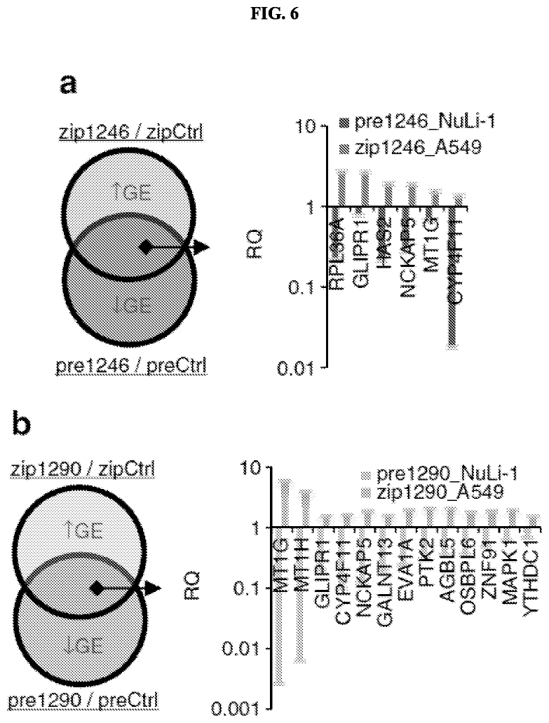

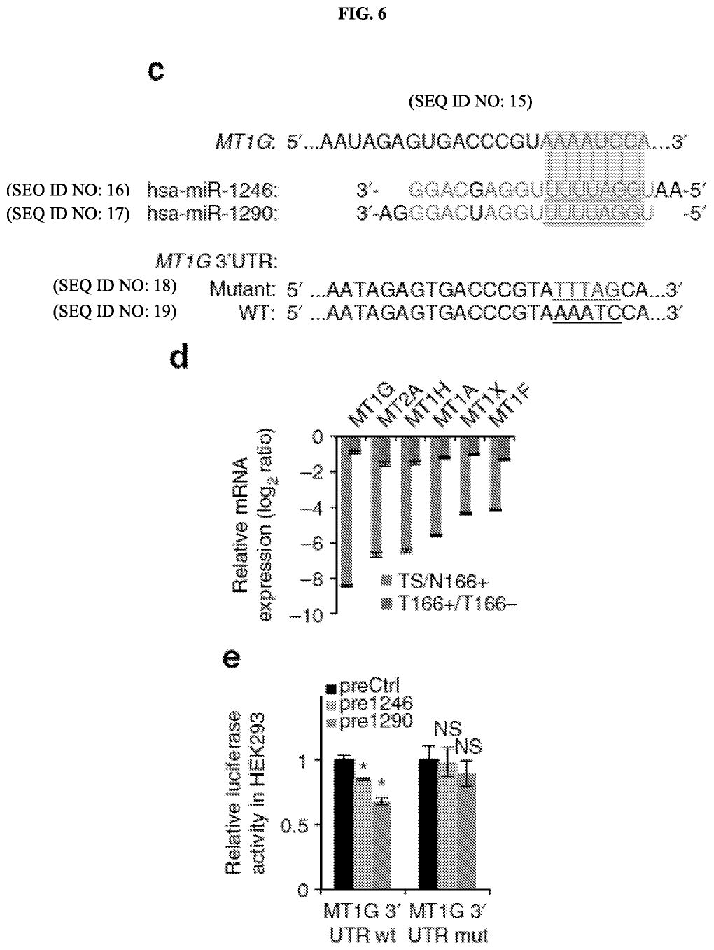

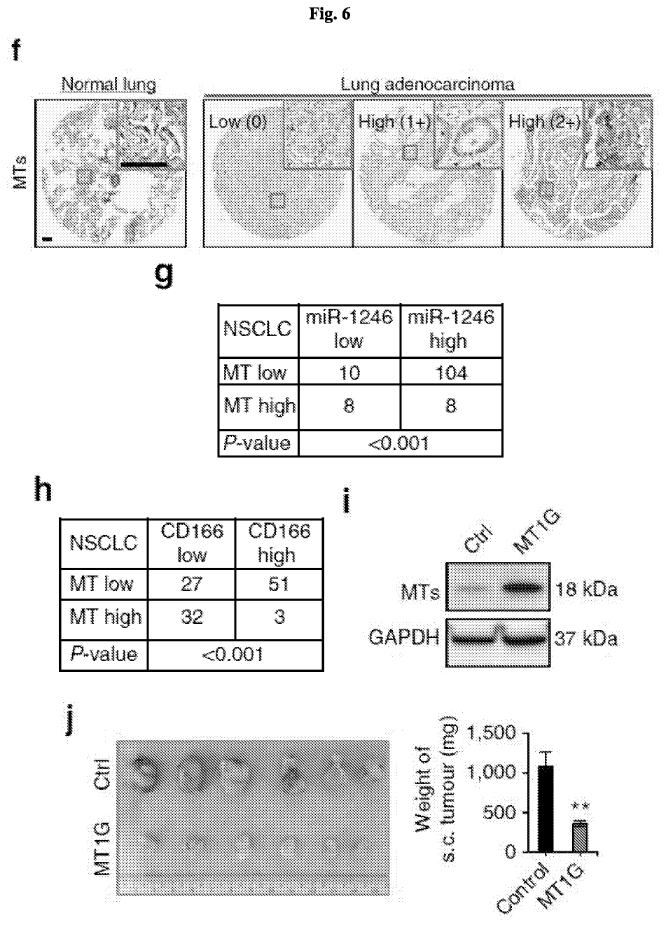

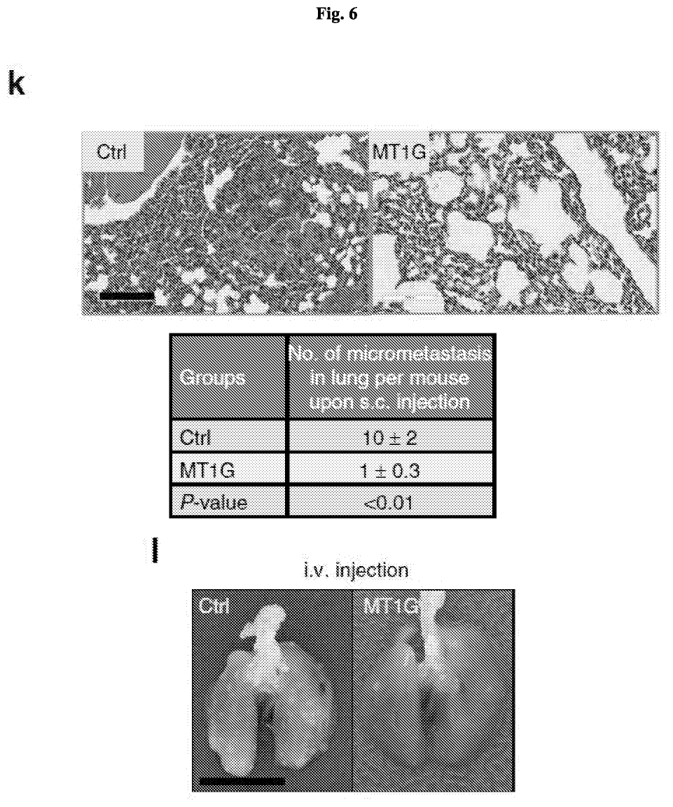

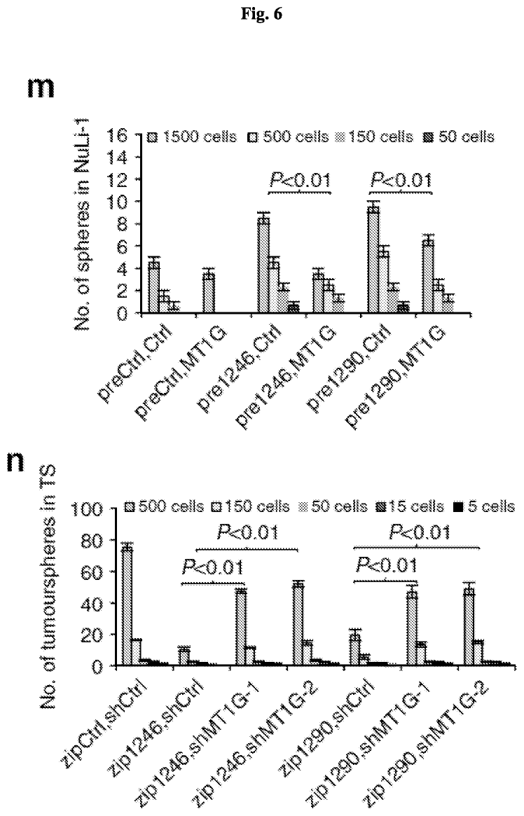

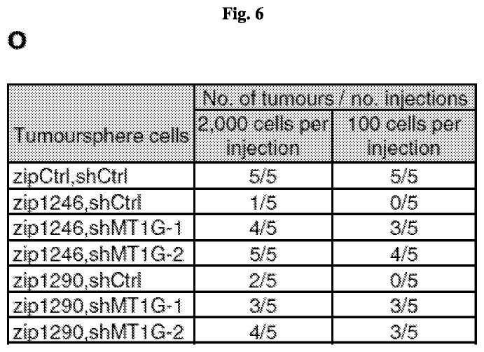

Because miRNAs are well-known to regulate the activities of downstream targets, which in turn, control the behaviour of a cell, this study sought to identify genes that might be directly targeted by miR-1246 or miR-1290. To do so, whole-transcriptome analyses were performed after knocking down miR-1246 or miR-1290 in A549, a metastatic lung cancer cell line, which expressed high levels of the miRNAs, as well as after overexpressing miR-1246 or miR-1290 in NHBE, a normal lung epithelial cell line not expressing the miRNAs. The upregulated genes on knockdown were intersected with genes downregulated on overexpression for each miRNA perturbation to gather a list of candidate mRNAs that could potentially be regulated by either miRNA. The study further validated these targets by qRT-PCR (FIG. 6a,b). Genes repressed by miR-1246 include PRL36A, GLIPR1, HAS2, NCKAP5, MT1G and CYP4F11, whereas miR-1290 represses MT1G, MT1H, GLIPR1, CYP4F11 and NCKAP5, among others. A few of the top target genes, such as MT1G and GLIPR1 were, in fact, common targets of both miR-1246 and miR-1290, thus suggesting that they may play an important role in mediating the function of lung cancer cells. Because the regulation of these gene expressions might be attributed to the indirect, secondary effects of miRNA perturbations, a computational approach to validate the interaction between miRNAs and their targets was taken. Using seed sequence base-pairing analyses, the study detected the duplex formations between human miR-1246 and miR-1290 with the 30-untranslated region (UTR) of mRNAs that include MT1G (FIG. 6c), thereby highlighting the propensity of both miRNAs to target a common gene.

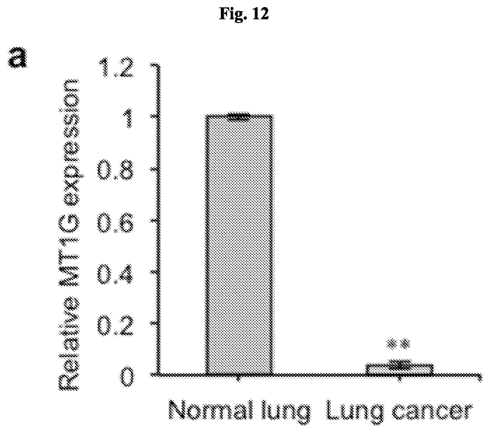

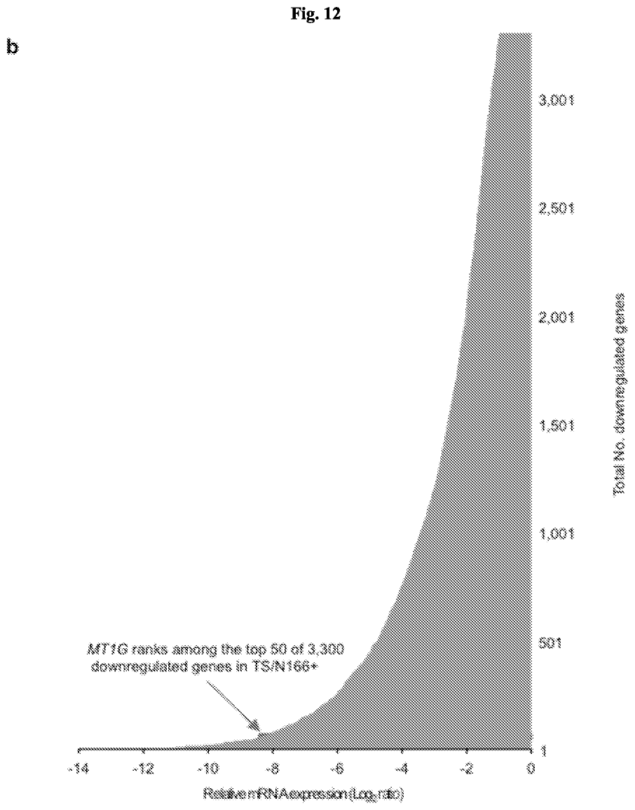

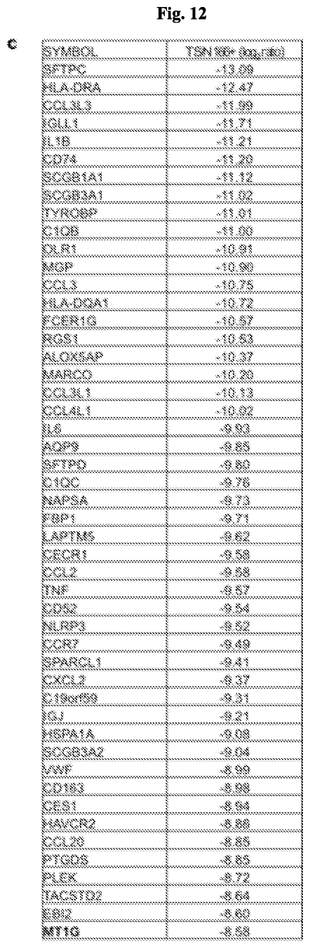

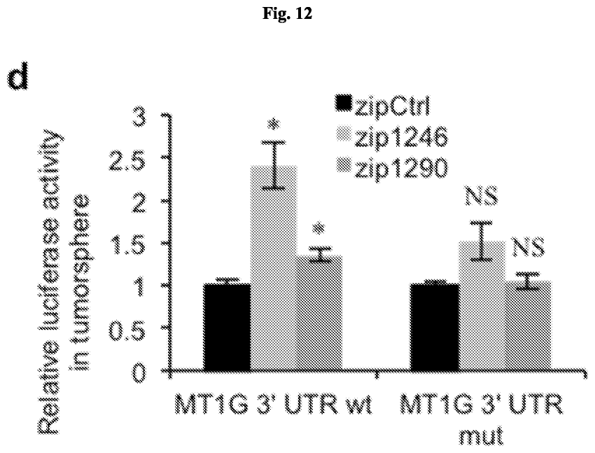

This study chose to focus on understanding the role of MT1G in lung TICs for several reasons. First, MT1G belongs to the metallothionein family of cysteine-rich metalloproteins which bind heavy metals. Metallothionein expression was reduced in several types of cancers, including lung cancer and hepatocellular carcinoma. MT1/2-knockout mice manifested an increased propensity for carcinogenesis. Second, in a cohort of NSCLC patients with paired tumour and normal tissues (n=9), MT1G was strongly expressed in normal tissues but markedly reduced in tumours (FIG. 12a). Third, transcriptome analysis comparing tumourspheres and CD166⁺ tumour cells with normal CD166⁺ lung epithelial cells and CD166⁻ tumour cells clearly placed MT1G among the top downregulated genes (FIG. 12b,c). Of genes in the metallothionein family, MT1G was the most highly suppressed in lung TICs (FIG. 6d).

To confirm MT1G as a direct target of miR-1246 and miR-1290, the study cloned its wild-type 30-UTR, as well as made point mutations, and tagged them to a luciferase reporter vector. On co-transfection of either miR-1246 or miR-1290 together with wild-type MT1G 3′-UTR reporters into HEK293 cells, the luciferase activities were reduced significantly (FIG. 6e). No change was observed for mutant MT1G 3′-UTR reporter. Conversely, knockdown of miR-1246 or miR-1290 in tumourspheres increased luciferase activity of wild-type MT1G 3′-UTR but not the mutant (FIG. 12d).

Similar to the expression patterns of miR-1246, miR-1290 and CD166, we also observed heterogeneous expression of metallothioneins within human lung tumour sections. Here, this study assessed the total levels of all metallothioneins because an antibody specific to MT1G was not available. While metallothioneins were abundant in normal lung tissues, their expression were varied across different adenocarcinomas, ranging from low to high (FIG. 6f and Table 7). Interestingly, well-differentiated tumours tended to express higher levels of metallothioneins, whereas poorly differentiated tumours did not. The study then sought to correlate the expression of metallothioneins, which was classified as low or high based on immunohistochemistry, with either miR-1246 or CD166 protein level in a cohort of patient tumours. metallothioneins expression was inversely correlated with both miR-1246 (FIG. 6g) and CD166 expression (FIG. 6h), providing an indication that metallothionein expression was repressed in lung TICs which tend to express the miR-1246 and CD166.

TABLE 7
Clinical pathologic characteristics of 143 study
subjects based on the expression intensity of MT.

| | Low | | High | | |
|---|---|---|---|---|---|
| Expression of MT | (n = 67) | | (n = 76) | | p-value |
| Mean age (yrs) | 61.4 | (10.9) | 65.5 | (8.7) | 0.014 |
| Gender (%) | | | | | |
| Male | 43 | (64) | 54 | (71) | 0.473 |
| Female | 24 | (36) | 22 | (29) | |
| Histology (%) | | | | | |
| Adenocarcinoma | 53 | (79) | 44 | (58) | 0.007 |
| SCC | 14 | (21) | 32 | (42) | |
| Mean tumour size (cm) | 3.87 | (2.18) | 3.73 | (1.88) | 0.684 |
| T stage (%) | | | | | |
| T1 | 11 | (17) | 22 | (31) | 0.001 |
| T2 | 28 | (44) | 43 | (60) | |
| T3 | 13 | (20) | 3 | (4) | |
| T4 | 12 | (19) | 4 | (6) | |
| N stage (%) | | | | | |
| N0 | 43 | (67) | 61 | (85) | 0.025 |
| N1-3 | 21 | (33) | 11 | (15) | |
| M stage (%) | | | | | |
| M0 | 60 | (94) | 72 | (100) | 0.047 |
| M1 | 4 | (6) | 0 | (0) | |
| AJCC stage (%) | | | | | |
| I | 25 | (40) | 56 | (79) | <0.001 |
| II | 18 | (29) | 6 | (8) | |
| III | 14 | (23) | 9 | (13) | |
| IV | 5 | (8) | 0 | (0) | |
| Grade (%) | | | | | |
| Well-differentiated | 4 | (6) | 5 | (7) | 0.059 |
| Moderately differentiated | 38 | (58) | 36 | (48) | |
| Poorly differentiated | 15 | (23) | 31 | (41) | |
| Undifferentiated | 8 | (12) | 3 | (4) | |
| Status (%) | | | | | |
| Alive | 32 | (48) | 34 | (45) | 0.912 |
| Lung cancer related death | 29 | (43) | 34 | (45) | |
| Non-lung cancer related death | 6 | (9) | 8 | (10) | |

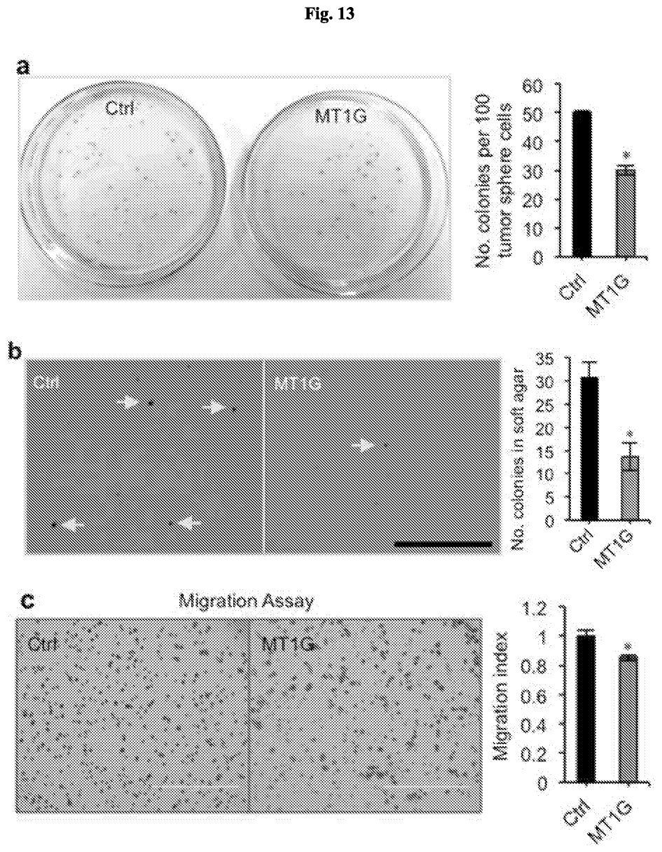

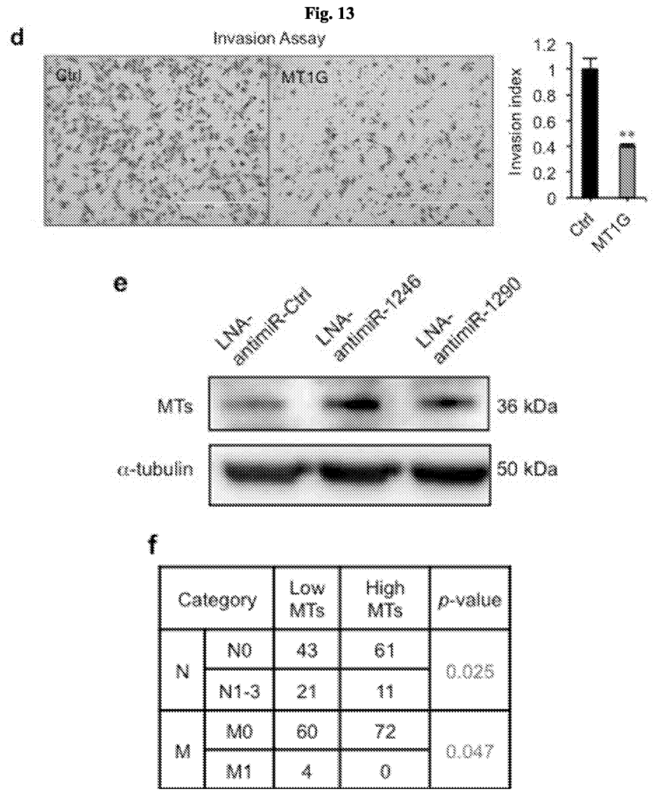

The above data indicated MT1G might have a role in the inhibition of tumour-initiation or metastasis. To test this, MT1G was first overexpressed in lung tumourspheres by lentiviral infection (FIG. 6i). Their abilities to form colonies on adherent cultures and on soft agar were inhibited markedly on MT1G overexpression (FIG. 13a,b). Their migration (FIG. 13c) and invasion capabilities in vitro (FIG. 13d) were, similarly, impaired. When patient tumourspheres overexpressing MT1G were xenografted subcutaneously into NSG mice, their growth were inhibited by at least threefold relative to control cells (FIG. 6j). Microscopic lung metastases arising from MT1G-expressing tumourspheres were also reduced significantly, as expected (FIG. 6k). To further test whether MT1G controls metastasis independent of tumour size, MT1G-expressing tumourspheres or control cells were introduced directly into the lung through tail-vein injection. The number of metastatic nodules was indeed reduced in the mice injected with MT1G-expressing tumourspheres relative to control cells (FIG. 6l). Interestingly, in mice bearing xenografted tumours that were treated with anti-miR-1246 or anti-miR-1290 LNAs (FIG. 5e), these small residual tumours had increased metallothionein protein level (FIG. 13e). It remains, however, unclear whether these contained differentiated non-TICs that are resistant to the TIC-specific LNAs, or the inhibition of miRNAs de-repressed the inhibition of MT1G expression. Clinically, in NSCLC patients, lower metallothionein protein expression was associated with regional lymph node invasion (P=0.025 by x-test) and distant metastasis (P=0.047 by χ²-test; FIG. 13f).

If MT1G was a major target of both miRNAs, the study tested whether MT1G overexpression could counter the effect of miR-1246 or miR-1290 expression. The first treated NuLi-1 cells with pre1246 or pre1290 and this led to an increase in their sphere-forming ability, as expected (FIG. 6m). However, in a separate experiment, when the cells were simultaneously overexpressing MT1G, their sphere-forming ability was inhibited. Conversely, the study also investigated the mechanistic regulation of MT1G and the miRNAs in TS cells. Knocking down either miR-1246 or miR-1290 markedly reduced the tumoursphere-forming ability of TS cells as anticipated (FIG. 6n). The concomitant depletion of MT1G in miR-1246- or miR-1290-depleted cells could, at least in part, rescue the effect of miRNA loss. Finally, the transplantation of these treated cells in limiting cell numbers into NSG mice clearly showed that the TS cells containing MT1G knock down remain tumorigenic even in the presence of miR-1246 or miR-1290 loss, thus confirming MT1G to be their major target (FIG. 6o). Taken altogether, the results indicate that miR-1246 and miR-1290, which are enriched in TICs, have critical roles in regulating tumour growth and metastasis, in part, through the repression of metallothioneins, especially MT1G.

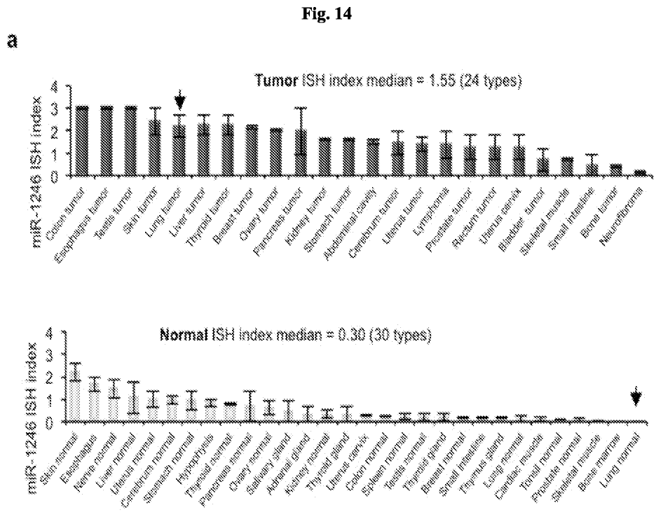

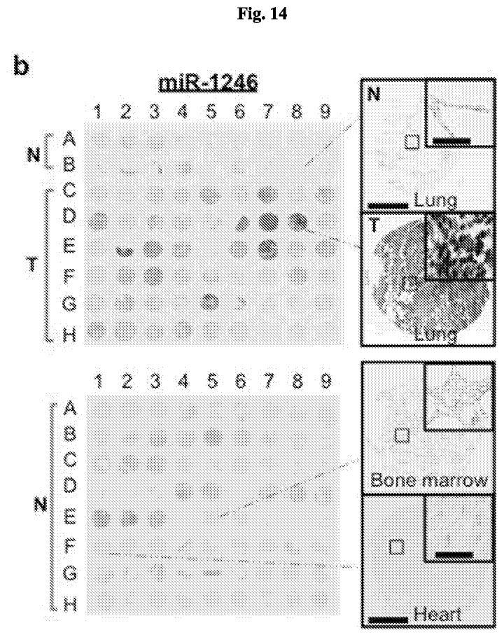

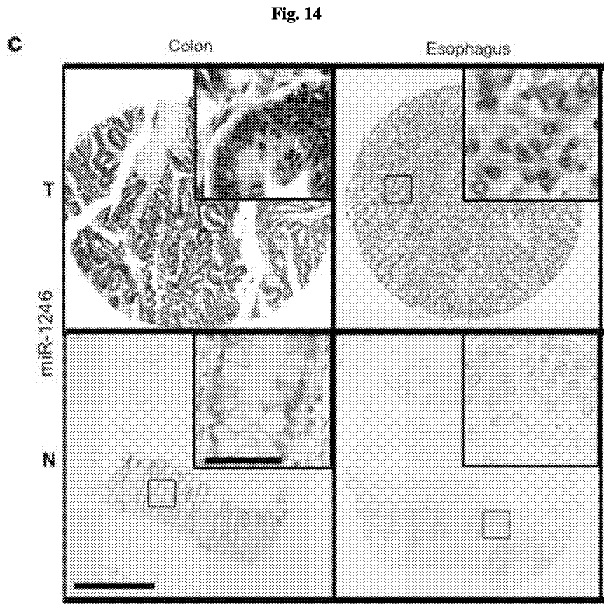

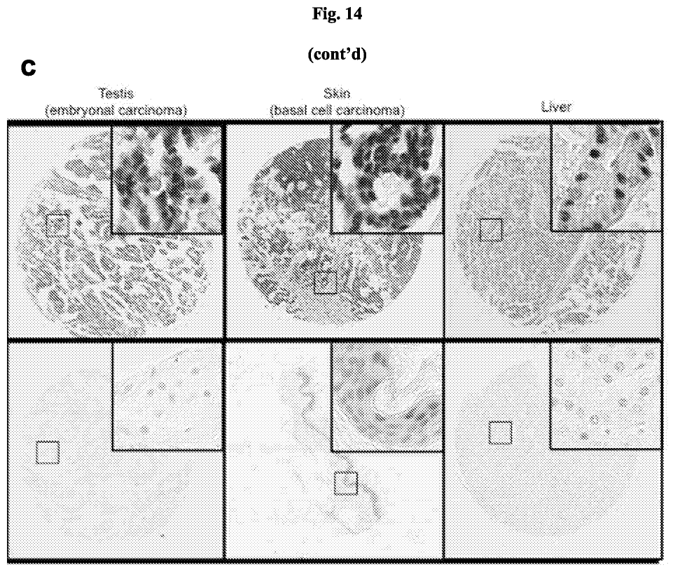

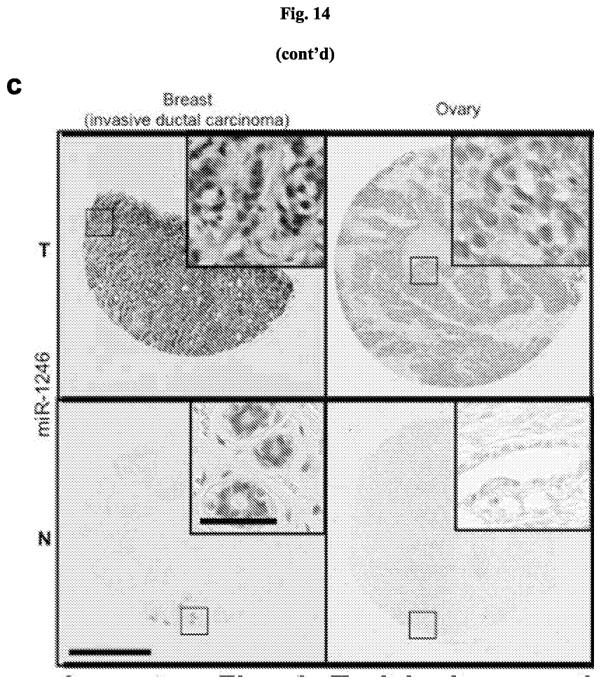

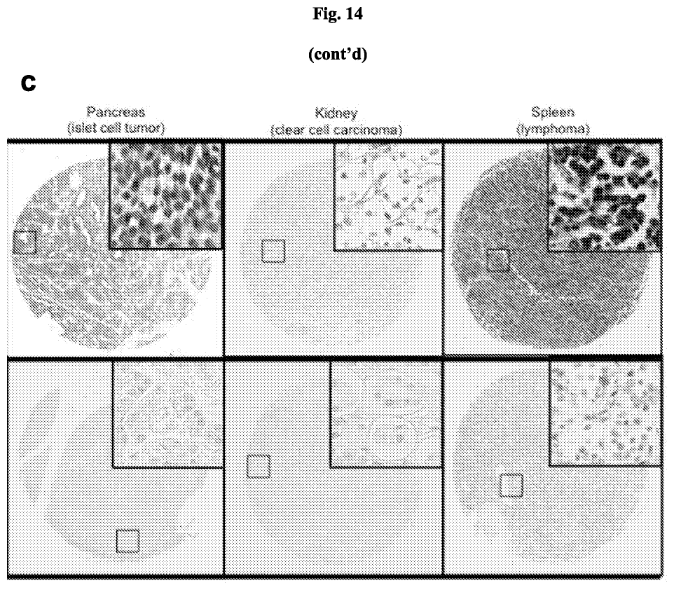

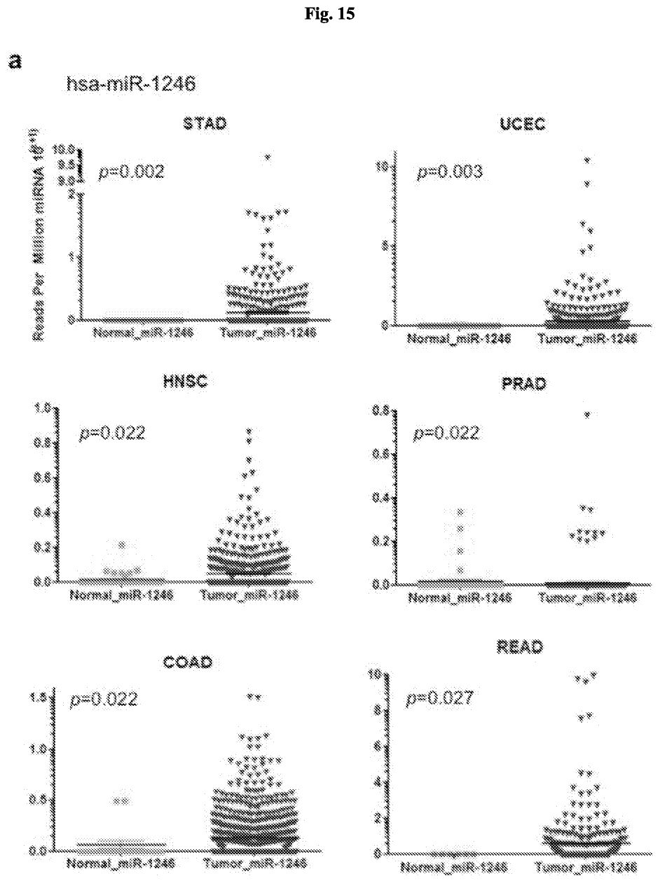

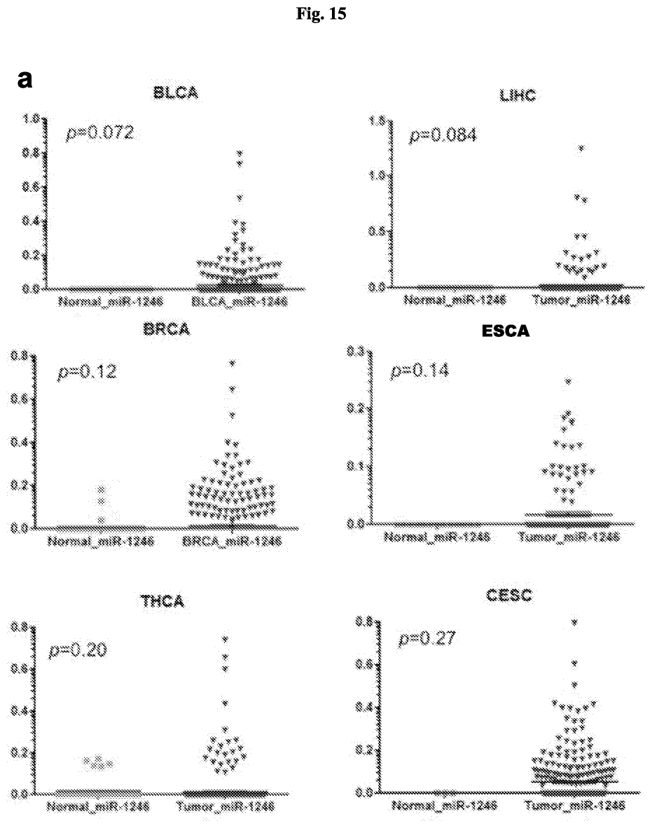

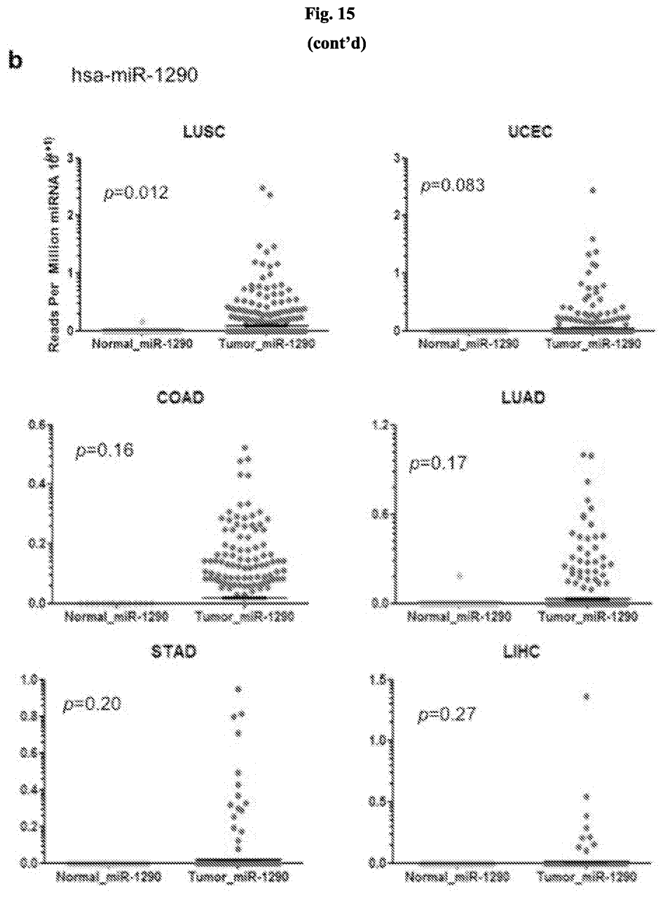

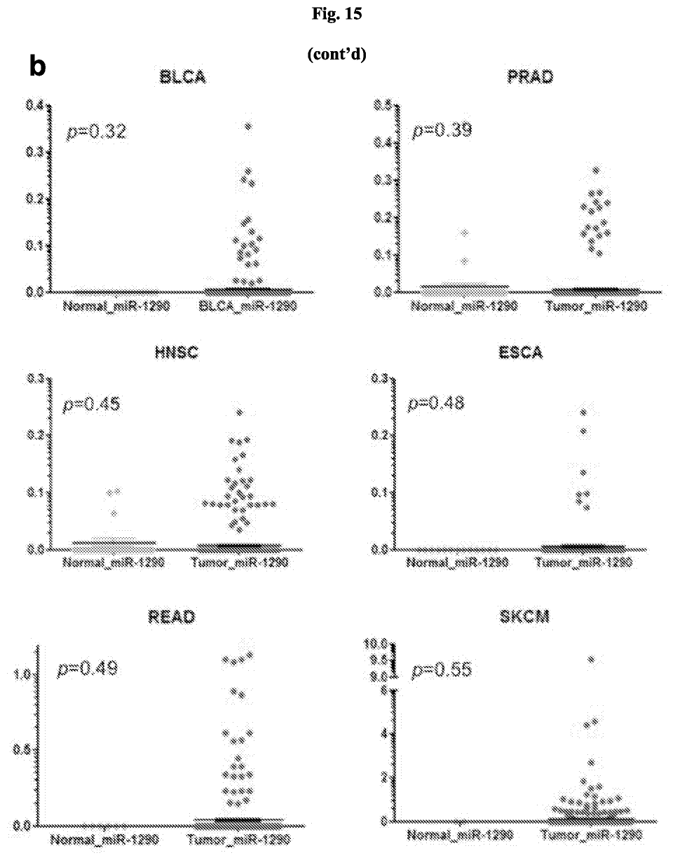

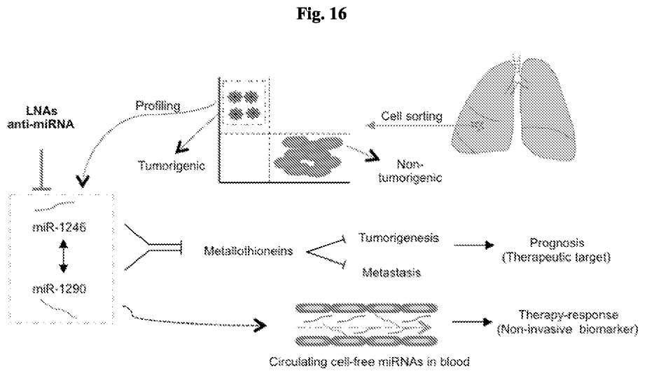

Using an unbiased approach to uncover TIC-specific miRNAs from patient-derived tumour cells and tumourspheres, this study identified a miRNA signature containing two miRNAs, miR-1246 and miR-1290, both of which contribute towards tumour initiation and metastasis. Interestingly, across the major types of cancers that include breast, colon, and head and neck, both miRNAs are strongly upregulated in tumours relative to their normal counterparts (FIGS. 14 and 15 and Tables 2 and 8-10). The observations indicate miR-1246 and miR-1290 can behave as non-invasive biomarkers that may be exploited for the early detection of a broad spectrum of cancers.

TABLE 8
Expression of miR-1246 and miR-1290 in a tissue microarray FDA808-2

| | | | | | | | miR-1246 | | miR-1290 | |
|---|---|---|---|---|---|---|---|---|---|---|
| | | | | | | | Int. | Pct. | Int. | Pct. |
| position | organ | pathology | grade | stage | tnm | type | (+) | (%) | (+) | (%) |
| A1 | Uterus | Normal endometrium tissue | — | — | — | N | 2 | 75 | 1 | 70 |
| A2 | Uterus | Normal endometrium tissue | — | — | — | N | 3 | 90 | 3 | 90 |
| A3 | Uterus | Normal endometrium tissue | — | — | — | N | 2 | 90 | 2 | 100 |
| A4 | Uterine | Cancer adjacent normal | — | — | — | NAT | 0 | 0 | 0 | 0 |
| | cervix | cervical canals tissue | | | | | | | | |
| A5 | Uterine | Normal cervical canals | — | — | — | N | 0 | 0 | 1 | 20 |
| | cervix | tissue | | | | | | | | |
| A6 | Uterus | Cancer adjacent normal | — | — | — | NAT | 1 | 5 | 0 | 0 |
| | | cervix tissue | | | | | | | | |
| A7 | Skeletal | Normal skeletal muscle | — | — | — | N | 1 | 80 | 0 | 0 |
| | muscle | tissue | | | | | | | | |
| A8 | Skeletal | Normal skeletal muscle | — | — | — | N | 0 | 0 | 0 | 0 |
| | muscle | tissue | | | | | | | | |
| A9 | Thyroid | Normal thyroid gland | — | — | — | N | 1 | 80 | 0 | 0 |
| | | tissue (with hyperplasia | | | | | | | | |
| | | in follicles) | | | | | | | | |
| B1 | Skin | Normal skin tissue | — | — | — | N | 2 | 90 | 0 | 0 |
| B2 | Skin | Normal skin tissue | — | — | — | N | 3 | 100 | 0 | 0 |
| B3 | Skin | Normal skin tissue | — | — | — | N | 2 | 90 | 2 | 80 |
| B4 | Nerve | Normal peripheral nerve | — | — | — | N | 2 | 80 | 2 | 5 |
| | | tissue | | | | | | | | |
| B5 | Nerve | Normal peripheral nerve | — | — | — | N | 1 | 70 | 0 | 0 |
| | | tissue | | | | | | | | |
| B6 | Nerve | Normal peripheral nerve | — | — | — | N | 3 | 70 | 0 | 0 |
| | | tissue | | | | | | | | |
| B7 | Lung | Normal lung tissue | — | — | — | N | 0 | 0 | 0 | 0 |
| | | (pneumonia) | | | | | | | | |
| B8 | Lung | Normal lung tissue | — | — | — | N | 0 | 0 | 0 | 0 |
| B9 | Lung | Normal lung tissue | — | — | — | N | 2 | 20 | 1 | 5 |
| C1 | Cerebrum | Glioblastoma | — | — | — | T | 2 | 80 | 0 | 0 |
| C2 | Cerebrum | Atypical meningioma | — | — | — | T | 1 | 70 | 1 | 75 |
| C3 | Cerebrum | Malignant ependymoma | — | — | — | T | 1 | 80 | 2 | 80 |
| C4 | Cerebrum | Malignant oligodendroglioma | — | — | — | T | 3 | 90 | 3 | 70 |
| C5 | Ovary | Serous adenocarcinoma | 3 | II | T2N0M0 | T | 2 | 100 | 2 | 100 |
| C6 | Ovary | Adenocarcinoma | 3 | III | T3N0M0 | T | 2 | 100 | 2 | 90 |
| C7 | Pancreas | Islet cell carcinoma | — | | | T | 3 | 100 | 3 | 100 |
| C8 | Pancreas | Adenocarcinoma | 3 | II | T2N0M0 | T | 1 | 90 | 2 | 90 |
| C9 | Testis | Seminoma | — | I | T1N0M0 | T | 3 | 100 | 3 | 100 |
| D1 | Testis | Embryonal carcinoma | — | I | T2N0M0 | T | 3 | 100 | 1 | 65 |
| D2 | Thyroid | Medullary carcinoma | — | II | T3N0M0 | T | 2 | 90 | n.a | n.a. |
| D3 | Thyroid | Papillary carcinoma | — | III | T2aN1M0 | T | 3 | 90 | 2 | 10 |
| D4 | Breast | Intraductal carcinoma | — | 0 | TisN0M0 | T | 2 | 80 | 2 | 100 |
| | | with early infiltrate | | | | | | | | |
| D5 | Breast | Invasive ductal carcinoma | 2 | IIa | T2N0M0 | T | 2 | 90 | 3 | 90 |
| D6 | Breast | Invasive ductal carcinoma | 2 | IIb | T2N1M0 | T | 3 | 100 | 3 | 100 |
| D7 | Spleen | Diffuse B-cell lymphoma | — | — | — | T | 3 | 100 | 3 | 100 |
| D8 | Lung | Small cell undifferentiated | — | I | T2N0M0 | T | 3 | 100 | 3 | 100 |
| | | carcinoma | | | | | | | | |
| D9 | Lung | Squamous cell carcinoma | 3 | I | T2N0M0 | T | 2 | 90 | 2 | 90 |
| E1 | Lung | Adenocarcinoma | 2 | II | T2N1M0 | T | 2 | 90 | 2 | 15 |
| E2 | Esophagus | Squamous cell carcinoma | 2 | IIa | T2N0M0 | T | 3 | 100 | 2 | 70 |
| E3 | Esophagus | Adenocarcinoma | 3 | IIa | T3N0M0 | T | 3 | 100 | 2 | 90 |
| E4 | Stomach | Mucinous adenocarcinoma | 3 | II | T2N1M0 | T | 2 | 80 | 3 | 90 |
| E5 | Small | Adenocarcinoma | 2 | II | T3N0M0 | T | 0 | 0 | n.a | n.a. |
| | intestine | | | | | | | | | |
| E6 | Small | Malignant interstitialoma | — | IIb | T2N0M0 | T | 1 | 90 | 0 | 0 |
| | intestine | | | | | | | | | |
| E7 | Colon | Adenocarcinoma | 2 | II | T4N0M0 | T | 3 | 100 | 3 | 100 |
| E8 | Abdominal | Interstitialoma | — | IIb | T2N0M0 | T | 2 | 80 | 2 | 80 |
| | cavity | | | | | | | | | |
| E9 | Rectum | Adenocarcinoma | 2 | I | T2N0M0 | T | 2 | 90 | 2 | 100 |
| F1 | Rectum | Intermediate grade | — | IIb | T2N0M0G2 | T | 1 | 70 | 0 | 0 |
| | | malignant interstitialoma | | | | | | | | |
| F2 | Liver | Hepatocellular carcinoma | 1 | III | T3N0M0 | T | 2 | 90 | 2 | 70 |
| F3 | Liver | Hepatoblastoma | — | — | — | T | 3 | 90 | 3 | 100 |
| F4 | Kidney | Clear cell carcinoma | 2 | I | T1N0M0 | T | 2 | 80 | 2 | 80 |
| F5 | Prostate | Adenocarcinoma | 3 | II | T2N0M0 | T | 2 | 90 | 0 | 0 |
| F6 | Prostate | Adenocarcinoma | 2 | II | T2N0M0G4 | T | 1 | 70 | 1 | 80 |
| F7 | Uterus | Leiomyoma | — | IIa | T1bN0M0 | BT | 1 | 80 | 1 | 80 |
| F8 | Uterus | Adenocarcinoma endometrium | 3 | IIa | T2aN0M0 | T | 2 | 90 | 2 | 100 |
| F9 | Uterus | Clear cell carcinoma with | — | IIb | T2bN0M0 | T | 2 | 90 | 2 | 90 |
| | | necrosis | | | | | | | | |
| G1 | Uterine | Squamous cell carcinoma | 3 | IIIb | T1bN1M0 | T | 1 | 70 | 1 | 40 |
| | cervix | | | | | | | | | |
| G2 | Uterine | Squamous cell carcinoma | 3 | II | T2N0M0 | T | 2 | 90 | 2 | 90 |
| | cervix | | | | | | | | | |
| G3 | Striated | Embryonal rhabdomyosarcom | — | Ia | T1aN0M0 | T | 1 | 70 | 1 | 5 |
| | muscle | of left leg | | | | | | | | |
| G4 | Rectum | Malignant melanoma | — | II | T4N0M0 | T | 1 | 60 | 1 | 40 |
| G5 | Skin | Basal cell carcinoma | — | I | T2N0M0 | T | 3 | 100 | 3 | 90 |
| | | of head | | | | | | | | |
| G6 | Skin | Squamous cell carcinoma | 2 | II | T3N0M0 | T | 2 | 90 | 2 | 80 |
| | | of left chest wall | | | | | | | | |
| G7 | Soft tissue | Neurofibroma | — | — | — | BT | 1 | 15 | 0 | 0 |
| G8 | Retroperitoneum | Neuroblastoma | — | IIIb | T2bN0M0 | T | 2 | 10 | 3 | 10 |
| G9 | Abdominal cavity | Malignant mesothelioma | — | I | T2N0M0 | T | 2 | 70 | 2 | 60 |
| H1 | Mediastinum | Diffuse B-cell lymphoma | — | — | — | T | 2 | 90 | 1 | 50 |
| H2 | Lymph node | Diffuse B cell lymphoma | — | — | — | T | 1 | 40 | 1 | 60 |
| | | of right thigh | | | | | | | | |
| H3 | Lymph node | Hodgkin's lymphoma | — | — | — | T | 1 | 50 | 1 | 80 |
| H4 | Pelvic cavity | Large cell anaplastic | — | — | — | T | 2 | 80 | 2 | 70 |
| | | lymphoma | | | | | | | | |
| H5 | Bladder | Transitional cell | — | II | T3aN0M0 | T | 2 | 60 | 2 | 75 |
| | | carcinoma | | | | | | | | |
| H6 | Bladder | Low grade malignant | — | Ib | T2bN0M0G1 | T | 1 | 20 | 1 | 60 |
| | | leiomyosarcoma | | | | | | | | |
| H7 | Bone | Osteosarcoma of right | — | IIIb | T2N0M0 | T | 1 | 40 | 2 | 60 |
| | | inferior extremity femur | | | | | | | | |
| H8 | Retroperitoneum | Spindle cell | — | IIb | T2N0M0G2 | T | 0 | 0 | 1 | 30 |
| | | rhabdomyosarcoma | | | | | | | | |
| H9 | Smooth muscle | Intermediate grade | — | IIIb | T2bN0M0 | T | 0 | 0 | 1 | 40 |
| | | malignant leiomyosarcoma | | | | | | | | |
| | | of left buttock | | | | | | | | |

Note:
N, normal; T, malignant tumor; NAT, cancer adjacent normal; n.a., not applicable; BT, benign tumor; Grade (1, well-differentiated; 2, moderately-differentiated; 3, poorly differentiated; 4, undifferentiated); TNM grading (T, primary tumor; N, regional lymph nodes; M, distant metastasis).

TABLE 9
Expression of miR-1246 and miR-1290 in a tissue microarray FDA808-1

| | | | | miR-1246 | | miR-1290 | |
|---|---|---|---|---|---|---|---|
| | | | | Int. | Pct. | Int. | Pct. |
| position | organ | pathology | type | (+) | (%) | (+) | (%) |
| A1 | Cerebrum | Normal cerebrum tissue | N | 1 | 50 | 0 | 0 |
| A2 | Cerebrum | Normal cerebrum tissue | N | 2 | 90 | 0 | 0 |
| A3 | Cerebrum | Normal cerebrum tissue | N | 3 | 20 | 0 | 0 |
| A4 | Cerebellum | Normal cerebellum tissue | N | 1 | 100 | 0 | 0 |
| A5 | Cerebellum | Normal cerebellum tissue | N | 1 | 100 | 0 | 0 |
| A6 | Cerebellum | Normal cerebellum tissue | N | 1 | 100 | 0 | 0 |
| A7 | Adrenal gland | Normal adrenal gland tissue | N | 0 | 0 | 0 | 0 |
| A8 | Adrenal gland | Normal adrenal gland tissue | N | 0 | 0 | 0 | 0 |
| A9 | Adrenal gland | Normal adrenal gland tissue | N | 1 | 100 | 2 | 75 |
| B1 | Ovary | Normal ovary tissue | N | 1 | 100 | 0 | 0 |
| B2 | Ovary | Normal ovary tissue | N | 0 | 0 | 0 | 0 |
| B3 | Ovary | Normal ovary tissue | N | 1 | 90 | 0 | 0 |
| B4 | Pancreas | Normal pancreas tissue | N | 1 | 5 | 1 | 80 |
| B5 | Pancreas | Normal pancreas tissue | N | 2 | 100 | 0 | 0 |
| B6 | Pancreas | Normal pancreas tissue | N | 0 | 0 | 0 | 0 |
| B7 | Parathyroid | Normal thyroid gland tissue | N | 1 | 100 | 3 | 80 |
| | gland | | | | | | |
| B8 | Parathyroid | Normal thyroid gland tissue | N | 0 | 0 | 2 | 70 |
| | gland | | | | | | |
| B9 | Parathyroid | Normal parathyroid gland tissue | N | 0 | 0 | 1 | 30 |
| | gland | | | | | | |
| C1 | Hypophysis | Normal hypophysis tissue | N | 1 | 100 | 0 | 0 |
| C2 | Hypophysis | Normal hypophysis tissue | N | 1 | 100 | 0 | 0 |
| C3 | Hypophysis | Normal hypophysis tissue | N | 1 | 50 | 0 | 0 |
| C4 | Testis | Normal testis tissue | N | 1 | 50 | 0 | 0 |
| C5 | Testis | Normal testis tissue | N | 0 | | 0 | 0 |
| C6 | Testis | Normal testis tissue | N | 1 | 10 | 0 | 0 |
| C7 | Thyroid gland | Normal thyroid gland tissue | N | 1 | 50 | 2 | 40 |
| C8 | Thyroid gland | Normal thyroid gland tissue | N | 1 | 10 | 2 | 50 |
| C9 | Thyroid gland | Normal thyroid gland tissue | N | 0 | | 2 | 10 |
| D1 | Breast | Normal breast tissue | N | 1 | 10 | 0 | 0 |
| D2 | Breast | Normal breast tissue | N | 2 | 10 | 0 | 0 |
| D3 | Breast | Normal breast tissue | N | 2 | 10 | 0 | 0 |
| D4 | Spleen | Normal spleen tissue | N | 2 | 10 | 0 | 0 |
| D5 | Spleen | Normal spleen tissue | N | 1 | 50 | 0 | 0 |
| D6 | Spleen | Normal spleen tissue | N | 0 | 0 | 2 | 70 |
| D7 | Tonsil | Normal tonsil tissue | N | 1 | 10 | 2 | 70 |
| D8 | Tonsil | Normal tonsil tissue | N | 1 | 10 | 2 | 80 |
| D9 | Tonsil | Normal tonsil tissue | N | 1 | 10 | 1; 2 | 60 |
| E1 | Thymus gland | Normal thymus gland tissue | N | 1 | 20 | 0 | 0 |
| E2 | Thymus gland | Normal thymus gland tissue | N | 1 | 20 | 0 | 0 |
| E3 | Thymus gland | Normal thymus gland tissue | N | 1 | 10 | 0 | 0 |
| E4 | Bone marrow | Normal myeloid tissue | N | 0 | 0 | 0 | 0 |
| E5 | Bone marrow | Normal myeloid tissue | N | 0 | 0 | 0 | 0 |
| E6 | Bone marrow | Normal myeloid tissue | N | 0 | 0 | 0 | 0 |
| E7 | Lung | Normal lung tissue | N | 0 | 0 | 2 | 10 |
| E8 | Lung | Normal lung tissue | N | 0 | 0 | 3 | 40 |
| E9 | Lung | Normal lung tissue | N | 0 | 0 | 0 | 0 |
| F1 | Heart | Normal cardiac muscle tissue | N | 1 | 30 | 0 | 0 |
| F2 | Heart | Normal cardiac muscle tissue | N | 0 | 0 | 0 | 0 |
| F3 | Heart | Normal cardiac muscle tissue | N | 1 | 5 | 0 | 0 |
| F4 | Esophagus | Normal esophagus tissue | N | 2 | 100 | 0 | 0 |
| F5 | Esophagus | Normal esophagus tissue | N | 2 | 100 | 1 | 20 |
| F6 | Esophagus | Normal esophagus tissue | N | 2 | 50 | 1 | 30 |
| F7 | Stomach | Normal stomach tissue | N | 2 | 50 | 2 | 20 |
| F8 | Stomach | Normal stomach tissue | N | 2 | 80 | 3 | 40 |
| F9 | Stomach | Normal stomach tissue | N | 1 | 20 | 2 | 30 |
| | | (smooth muscle tissue) | | | | | |
| G1 | Small intestine | Normal small intestine tissue | N | 1 | 10 | 1 | 60 |
| G2 | Small intestine | Normal small intestine tissue | N | 2 | 10 | 1 | 60 |
| G3 | Small intestine | Normal small intestine tissue | N | 2 | 10 | 2 | 75 |
| G4 | Colon | Normal colon tissue | N | 2 | 10 | 0 | 0 |
| G5 | Colon | Normal colon tissue | N | 3 | 10 | 2 | 10 |
| G6 | Colon | Normal colon tissue | N | 2 | 10 | 1 | 25 |
| G7 | Liver | Normal liver tissue | N | 1 | 80 | 1 | 80 |
| G8 | Liver | Normal liver tissue | N | 0 | 0 | 2 | 75 |
| G9 | Liver | Normal liver tissue | N | 3 | 80 | 3 | 90 |
| H1 | Salivary gland | Normal salivary gland tissue | N | 2 | 70 | 0 | 0 |
| H2 | Salivary gland | Normal salivary gland tissue | N | 0 | 0 | 1 | 15 |
| H3 | Salivary gland | Normal salivary gland tissue | N | 0 | 0 | 0 | 0 |
| H4 | Kidney | Normal kidney tissue | N | 0 | 0 | 1 | 10 |
| H5 | Kidney | Normal kidney tissue | N | 1 | 50 | 2 | 15 |
| H6 | Kidney | Normal kidney tissue | N | 1 | 50 | 1 | 10 |
| H7 | Prostate | Normal prostate tissue | N | 0 | 0 | 0 | 0 |
| H8 | Prostate | Normal prostate tissue | N | 0 | 0 | 2 | 90 |
| H9 | Prostate | Normal prostate tissue | N | 2 | 10 | 2 | 90 |

Note:
N, normal; Int., intensity; Pct., percentage; n.a., not applicable.

TABLE 10
Expression of miR-1246 and miR-1290 in eight types
of cancers by ISH in a tissue microarray BC00112

| | | | | | miR-1246 | | miR-1290 | |
|---|---|---|---|---|---|---|---|---|
| | | | | | Int. | Pct. | Int. | Pct. |
| position | organ | pathology | grade | type | (+) | (%) | (+) | (%) |
| A1 | Liver | Hepatocellular carcinoma | I | T | 3 | 90 | 2 | 90 |
| A2 | Colon | Adenocarcinoma | I | T | 3 | 90 | 2 | 70 |
| A3 | Stomach | Adenocarcinoma | II | T | 2 | 60 | 0 | 0 |
| A4 | Esophagus | Squamous cell carcinoma | I | T | 3 | 70 | 2 | 20 |
| A5 | Intestine | Adenocarcinoma | I | T | 2 | 90 | 0 | 0 |
| A6 | Pancreas | Cancer adjacent tissue | — | N | 0 | 0 | 0 | 0 |
| A7 | Lung | Squamous cell carcinoma | II | T | 1 | 20 | 0 | 0 |
| A8 | Epiploon | Metastatic mucinous | II | Mets | 0 | 0 | 0 | 0 |
| | | adenocarcinoma | | | | | | |
| B1 | Liver | Hepatocellular carcinoma | II | T | 2 | 90 | 0 | 0 |
| B2 | Colon | Adenocarcinoma | III | T | 1 | 90 | 0 | 0 |
| B3 | Stomach | Adenocarcinoma | III | T | 2 | | 1 | 20 |
| B4 | Esophagus | Squamous cell carcinoma | II | T | 3 | 90 | 2 | 20 |
| B5 | Intestine | Adenocarcinoma | II | T | 1 | 90 | 0 | 0 |
| B6 | Pancreas | Duct adenocarcinoma | II | T | 2 | 80 | 0 | 0 |
| B7 | Lung | Squamous cell carcinoma | III | T | 3 | 80 | 1 | 10 |
| B8 | Epiploon | Metastatic adenocarcinoma | III | Mets | n.a. | n.a. | n.a. | n.a. |
| C1 | Liver | Hepatocellular carcinoma | II | T | n.a. | n.a. | n.a. | n.a. |
| C2 | Colon | Mucinous adenocarcinoma | III | T | 0 | 0 | 0 | 0 |
| C3 | Stomach | Adenocarcinoma | III | T | 3 | 50 | 0 | 0 |
| C4 | Esophagus | Squamous cell carcinoma | II | T | 2 | 50 | 1 | 80 |
| C5 | Intestine | Adenocarcinoma | III | T | 1 | 40 | 0 | 0 |
| C6 | Pancreas | Islet cell carcinoma | — | T | 2 | 20 | 0 | 0 |
| C7 | Lung | Atypical carcinoid | — | T | 2 | 20 | 1 | 5 |
| C8 | Epiploon | Metastatic adenocarcinoma | III | Mets | 0 | 0 | 0 | 0 |
| D1 | Liver | Cancer adjacent normal | — | N | 2 | 80 | 0 | 0 |
| | | hepatic tissue | | | | | | |
| D2 | Colon | Cancer adjacent normal | — | N | 3 | 30 | 0 | 0 |
| | | colonic tissue | | | | | | |
| D3 | Stomach | Cancer adjacent normal | — | N | 1 | 10 | 0 | 0 |
| | | gastric tissue | | | | | | |
| D4 | Esophagus | Cancer adjacent normal | — | N | 0 | 0 | 0 | 0 |
| | | esophageal tissue | | | | | | |
| D5 | Intestine | Cancer adjacent normal | — | N | 2 | 90 | 0 | 0 |
| | | small intestinal tissue | | | | | | |
| D6 | Pancreas | Cancer adjacent normal | — | N | 1 | 30 | 0 | 0 |
| | | pancreatic tissue | | | | | | |
| D7 | Lung | Cancer adjacent normal lung | — | N | 0 | 0 | 0 | 0 |
| | | tissue | | | | | | |
| D8 | Epiploon | Cancer adjacent normal | — | N | 0 | 0 | 0 | 0 |
| | | greater omentum tissue | | | | | | |
| E1 | Liver | Cancer adjacent normal | — | N | 1 | 90 | 0 | 0 |
| | | hepatic tissue | | | | | | |
| E2 | Colon | Cancer adjacent normal | — | N | 2 | 90 | 0 | 0 |
| | | colonic tissue | | | | | | |
| E3 | Stomach | Cancer adjacent normal | — | N | 0 | 0 | 0 | 0 |
| | | gastric tissue | | | | | | |
| E4 | Esophagus | Cancer adjacent normal | — | N | 2 | 80 | 0 | 0 |
| | | esophageal tissue | | | | | | |
| E5 | Intestine | Cancer adjacent normal | — | N | n.a. | n.a. | n.a. | n.a. |
| | | small intestinal tissue | | | | | | |
| E6 | Pancreas | Cancer adjacent normal | — | N | 1 | 10 | 0 | 0 |
| | | pancreatic tissue | | | | | | |
| E7 | Lung | Cancer adjacent normal lung | — | N | 0 | 0 | 0 | 0 |
| | | tissue | | | | | | |
| E8 | Epiploon | Cancer adjacent normal | — | N | 0 | 0 | 0 | 0 |
| | | greater omentum tissue | | | | | | |
| F1 | Liver | Cancer adjacent normal | — | N | 2 | 80 | 0 | 0 |
| | | hepatic tissue | | | | | | |
| F2 | Colon | Cancer adjacent colonic | — | N | 1 | 80 | 0 | 0 |
| | | mucosa tissue (chronic | | | | | | |
| | | inflammation) | | | | | | |
| F3 | Stomach | Cancer adjacent normal | — | N | 0 | 0 | 0 | 0 |
| | | gastric tissue | | | | | | |
| F4 | Esophagus | Cancer adjacent normal | — | N | 3 | 80 | 2 | 70 |
| | | esophageal tissue | | | | | | |
| F5 | Intestine | Cancer adjacent normal | — | N | 1 | 70 | 0 | 0 |
| | | small intestinal tissue | | | | | | |
| F6 | Pancreas | Cancer adjacent normal | — | N | 0 | 0 | 0 | 0 |
| | | pancreatic tissue | | | | | | |
| F7 | Lung | Cancer adjacent normal lung | — | N | 0 | 0 | 0 | 0 |
| | | tissue | | | | | | |
| F8 | Epiploon | Cancer adjacent normal | — | N | 0 | 0 | 0 | 0 |
| | | greater omentum tissue | | | | | | |

Note:
N, normal; T, malignant tumor; Mets, metastasis; Int., intensity; Pct., percentage; n.a., not applicable.

1-45. (canceled)

46. A method for determining the presence of lung cancer in a subject, the method comprising:
detecting an expression level of miR-1246 in a sample obtained from the subject; and
comparing the expression level of miR-1246 in the sample to an expression level of miR-1246 in a control sample,
wherein an increased expression level of miR-1246 in the sample obtained from the subject relative to the expression level of miR-1246 in the control sample indicates the presence of lung cancer in the subject.

47. The method according to claim 46, wherein the lung cancer is a non-small cell lung cancer, optionally wherein the lung cancer is early stage lung cancer, wherein the lung cancer is a stage I, II, III or IV lung cancer, optionally wherein the lung cancer is metastatic lung cancer.

48. The method of claim 46, wherein the detecting comprises any one of quantitative RT-PCR, in-situ hybridization, microRNA microarray or microRNA sequencing, optionally wherein the increased expression level is between a 1 to 20-fold increase, such as a 1-fold, 2-fold, 3-fold, 4-fold or 5-fold increase.

49. The method of claim 46, wherein the sample is selected from a tissue sample and a bodily fluid, optionally wherein the tissue sample is a lung tissue sample, optionally wherein the bodily fluid is selected from blood, urine, sputum, saliva, mucus, and semen, optionally wherein the blood sample is a serum or plasma sample.

50. The method of claim 46, further comprising:
detecting an expression level of miR-1290 in the sample obtained from the subject; and
comparing the expression level of miR-1290 in the sample to an expression level of miR-1290 in a control sample,
wherein an increased expression level of miR-1290 in the sample obtained from the subject relative to the expression level of miR-1290 in the control sample indicates the presence of lung cancer in the subject.

51. A method of monitoring a response to therapy in a lung cancer patient, comprising:
detecting an expression level of miR-1246, in a first sample obtained from the patient at a first time point;
detecting the expression level of miR-1246, in one or more further samples obtained from the patient at one or more further time points; and
comparing the expression level of miR-1246 detected at the first time point and one or more further time points,
wherein the expression level of miR-1246 in the one or more further samples relative to the expression level of miR-1246 in the first sample, indicates the patient's response to therapy.

52. The method according to claim 51, wherein the response is monitored throughout the course of therapy, optionally wherein the first time point is prior to the start of therapy, optionally wherein the one or more further time points are during the therapy and/or upon completion of the therapy.

53. The method according to claim 51, wherein an increase in the expression level of miR-1246 in the one or more further samples relative to the expression level of miR-1246 in the first sample, indicates a decreased response to therapy, optionally wherein the increased expression level is between a 1 to 20-fold increase, such as a 1-fold, 2-fold, 3-fold, 4-fold or 5-fold increase.

54. The method according to claim 51, wherein a decrease in the expression level of miR-1246 in the one or more further samples relative to the expression level of miR-1246 in the first sample, indicates an increased response to therapy, optionally wherein the decreased expression level is between a 1 to 20-fold decrease, such as a 1-fold, 2-fold, 3-fold, 4-fold or 5-fold decrease.

55. The method according to claim 51, wherein the therapy is an anti-cancer therapy selected from the group consisting of a chemotherapeutic treatment, immunotherapy, tyrosine-kinase inhibitor (TKI) therapy, a surgical treatment, a treatment with radiation therapy or any combination thereof, optionally wherein the chemotherapeutic treatment comprises treatment with an antimetabolite, platinum complex, spindle poison, DNA crosslinking drug and alkylating agent, bleomycin, antibiotic, and topoisomerase inhibitor or combinations thereof, optionally wherein the tyrosine-kinase inhibitor (TKI) therapy comprises treatment with an EGFR tyrosine kinase inhibitor (TKI).

56. The method of claim 51, wherein the lung cancer is a non-small cell lung cancer.

57. The method of claim 51, further comprising:
detecting an expression level of miR-1290, in the first sample obtained from the patient at the first time point;
detecting an expression level of miR-1290, in the one or more further samples obtained from the patient at one or more further time points; and
comparing the expression level of miR-1290 detected at the first time point and one or more further time points,
wherein the expression level of miR-1290 in the one or more further samples relative to the expression level of miR-1290 in the first sample, indicates the patient's response to therapy.

58. The method according to claim 51, wherein an increase in the expression level of miR-1290 in the one or more further samples relative to the expression level of miR-1290 in the first sample, indicates a decreased response to therapy, optionally wherein the increased expression level is between a 1 to 20-fold increase, such as a 1-fold, 2-fold, 3-fold, 4-fold or 5-fold increase.

59. The method according to claim 51, wherein a decrease in the level of miR-1290 in the one or more further samples relative to the expression level of miR-1290 in the first sample, indicates an increased response to therapy, optionally wherein the decreased expression level is between a 1 to 20-fold decrease, such as a 1-fold, 2-fold, 3-fold, 4-fold or 5-fold decrease.

60. A method of prognosis of lung cancer in a patient, comprising:
detecting an expression level of miR-1246 in a sample obtained from the subject; and
comparing the expression level of miR-1246 in the sample to an expression level of miR-1246 in a control sample,
wherein the expression level of miR-1246 in the sample obtained from the subject relative to the expression level of miR-1246 in the control sample indicates the prognosis of lung cancer in the subject.

61. The method of claim 60, wherein an increased expression level of miR-1246 in the sample obtained from the subject relative to the expression level of miR-1246 in the control sample indicates any one of a decreased overall survival, a decreased progression-free survival, a decreased relapse-free survival, and/or a decreased distant-metastasis free survival, optionally wherein the increased expression level is between a 1 to 20-fold increase, such as a 1-fold, 2-fold, 3-fold, 4-fold or 5-fold increase.

62. The method of claim 60, wherein the decreased expression level of miR-1246 in the sample obtained from the subject relative to the expression level of miR-1246 in the control sample indicates any one of an increased overall survival, an increased progression-free survival, an increased relapse-free survival, and/or an increased distant-metastasis free survival, optionally wherein the decreased expression level is between a 1 to 20-fold decrease, such as a 1-fold, 2-fold, 3-fold, 4-fold or 5-fold decrease.

63. A method for treating lung cancer in a subject, comprising administering to the subject one or more inhibitors of miR-1246.

64. The method of claim 63, wherein the one or more inhibitors of miR-1246 comprise an antisense oligonucleotide specific for miR-1246, optionally wherein the antisense oligonucleotide is a Locked Nucleic Acid (LNA) specific for miR-1246, optionally wherein the one or more inhibitors of miR-1246 is administered by any one of subcutaneous injection, intraperitoneal injection or intravenous injection.

65. The method of claim 63, further comprising administering to the subject one or more inhibitors of miR-1290, optionally wherein the one or more inhibitors of miR-1290 comprise an antisense oligonucleotide specific for miR-1290, optionally wherein the antisense oligonucleotide is a Locked Nucleic Acid (LNA) specific for miR-1290.
